SEQUENCE LISTING (17 sequences)

SEQ ID NO: 1 - protein, 1455 aa, Homo sapiens
MGGACSSFTTSSSPTIYSTSVTDSKAMQVESCSSAVGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAAAGALNVNVKKEISSPARPCSFEEAMKAMKTTGCNLDKVNIIPNALMTPLIPSSMIKSEDVTPMEVTAEKRSSTIFKTTKSVGSTQQTLENISNIAGNGSFSSPSSSHLPSENEKQQQIQPKAYNPETLTTIQTQDISQPGTFPAVSASSQLPNSDALLQQATQFQTRETQSREILQSDGTVVNLSQLTEASQQQQQSPLQEQAQTLQQQISSNIFPSPNSVSQLQNTIQQLQAGSFTGSTASGSSGSVDLVQQVLEAQQQLSSVLFSAPDGNENVQEQLSADIFQQVSQIQSGVSPGMFSSTEPTVHTRPDNLLPGRAESVHPQSENTLSNQQQQQQQQQQVMESSAAMVMEMQQSICQAAAQIQSELFPSTASANGNLQQSPVYQQTSHMMSALSTNEDMQMQCELFSSPPAVSGNETSTTTTQQVATPGTTMFQTSSSGDGEETGTQAKQIQNSVFQTMVQMQHSGDNQPQVNLFSSTKSMMSVQNSGTQQQGNGLFQQGNEMMSLQSGNFLQQSSHSQAQLFHPQNPIADAQNLSQETQGSLFHSPNPIVHSQTSTTSSEQMQPPMFHSQSTIAVLQGSSVPQDQQSTNIFLSQSPMNNLQTNTVAQEAFFAAPNSISPLQSTSNSEQQAAFQQQAPISHIQTPMLSQEQAQPPQQGLFQPQVALGSLPPNPMPQSQQGTMFQSQHSIVAMQSNSPSQEQQQQQQQQQQQQQQQQQSILFSNQNTMATMASPKQPPPNMIFNPNQNPMANQEQQNQSIFHQQSNMAPMNQEQQPMQFQSQSTVSSLQNPGPTQSESSQTPLFHSSPQIQLVQGSPSSQEQQVTLFLSPASMSALQTSINQQDMQQSPLYSPQNNMPGIQGATSSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 2 - DNA, 5304 bp, Homo sapiens
ggcggtggcggcgaccgtcagttttcgctgaggagaaacacgaaacggaccctttggctctcccccttccccttccccgtcctgaacccctctcctggtcaccgagaatcagtccccgtggagttccccctccacctcgccatcgtttcctcggtcctcggcccagtggaagtcactaccctcgaggaggaggcagcggcagccgccctcgcgtcgccgcccccggttcggtgcccgcggtcccggagaggaggtgccgccgccaccgccgctccccccctcccgctgccctcgggccgggctgggtcgagctgcgatgccctcggacttcatctcattgctcagcgcggacctagacctggaatcgcccaagtccctctactcgcgagaatctgtctatgatcttctcccaaaggagttacagttacctccatctagagaaacatctgtagcatcaatgagtcagacaagcggtggtgaggcaggctcgcctcctccagctgttgttgctgctggatttgcctctgaagcagggagtgtctgcattaaaaatgacctgtagttctctgcttcatagatgcttcttcagctccctcctcttcctccatgggcggtgcttgcagctcctttaccacctcttccagccctaccatttattctacctcagtcaccgacagcaaggctatgcaagtggagagctgctcctcagccgtgggggtaagtaacagaggggtaagtgaaaagcagttaaccagtaacacagttcagcagcatccatcaacaccgaagaggcacacagtcttgtacatctcaccaccacctgaggacttgctggataacagtcggatgtcctgccaggatgaggggtgtggattggaatctgagcagagctgcagtatgtggatggaggattccccctccaacttcagtaacatgagcaccagttcctacaatgataacactgaggtacctcgtaaatcacgaaaacgaaatccaaagcagaggccgggggtcaaacgacgagattgtgaagaatctaatatggatatatttgatgccgacagtgccaaagcacctcactatgtgctttctcagcttaccacggacaacaaaggcaactcaaaagcgggaaatggaacattggaaaaccaaaaaggaactggagtaaagaagagccctatgttgtgtggacaatatcctgttaaaagtgagggaaaggagctgaagatagttgtacaacctgagacacagcaccgagctcggtacctgactgagggcagccgtggctcagtgaaagatagaacacagcaaggctttcctacagtaaagctggaaggccataatgaacctgtagtgttgcaagtgtttgtgggcaacgactctggacgagtgaaaccacatggattttatcaggcctgcagagtaactggacgaaatacaactccttgcaaagaagtggacattgaaggcactactgttatagaagtcggccttgatcctagcaacaacatgacactggcggtggactgcgtagggatattgaaattgaggaatgctgatgtcgaagccagaataggaattgctggttccaagaagaaaagcactcgtgccagattggtttttcgagttaatatcatgaggaaagatggctccactttgacactgcaaacaccctcttctccaattttgtgtactcagccagcaggagtgccagaaatcttaaagaaaagcttgcatagctgttcagtgaaaggagaagaagaagtgtttttaatcggcaagaactttctgaaaggaactaaagttattttccaagaaaatgtttctgatgaaaactcttggaagtcagaagctgaaattgatatggaactatttcatcagaatcatcttattgtgaaggttcctccctatcatgaccaacatataactttgcctgtgtcagtgggaatatatgtagtgacaaatgctggaagatctcatgatgttcaaccattcacttacactccagacccagcagcagctggtgctttgaatgtaaatgtgaagaaggaaatatctagtccagcaagaccttgctcttttgaagaggccatgaaagcaatgaaaactactggatgtaatttagataaggtaaatattatccctaatgccctgatgactccactcataccaagcagtatgattaagagtgaagatgttactccaatggaagtaacagcagaaaaaagatcttccactatttttaagactacaaagtctgttggatcaactcagcaaacattagaaaacatctcaaacatagcaggaaatggctctttttcatcaccatcatcttcccacctaccttctgaaaatgaaaaacagcagcagattcagcccaaggcatacaacccagagaccctgacaactattcaaacccaggacatctcacagcctggtacttttccagcagtttctgcttctagtcagctgcccaacagcgatgcactattgcagcaggctacacagtttcagacaagagaaactcagtctagagagatattacagtcagatggtacagtggttaatttgtcacaactgactgaggcatcacaacaacagcagcagtcaccactacaagaacaagcacagactttacagcagcagatttcatcaaatatttttccatcaccaaatagtgtgagtcagcttcagaatactattcagcagctgcaagcagggagtttcacaggcagtactgctagtggcagcagtggaagtgttgacttggtccaacaagttttagaggcacagcagcagttatcttcagttttattttctgctccagatggtaatgagaatgttcaagagcagcttagtgcagatatttttcaacaagtcagtcaaattcagagtggtgtaagccctggaatgttttcctcaacagagccaacagtccataccagaccagataatttattacctggaagagctgaaagtgttcatccacagtctgaaaacacgttatctaatcaacagcagcagcagcagcagcaacagcaagtgatggaatcttcagccgcaatggtgatggagatgcaacagagtatctgccaggcagctgcccagattcagtcagagttattcccttcaactgcttcagcaaatggaaaccttcagcaatcgccagtttaccagcagacttctcacatgatgagtgcattgtctaccaatgaggatatgcaaatgcagtgtgaattgttttcttctcctcctgcagtttctggaaatgaaacttctacaactaccacacagcaggttgcaacccctggcactaccatgtttcagacatcaagttcaggagatggagaagaaactggaacacaagcaaaacagattcagaacagtgtctttcagaccatggtccaaatgcaacatagtggggacaatcaacctcaagttaaccttttttcatccacaaaaagtatgatgagtgttcagaatagtggtacccaacaacaaggtaatggtttattccagcaagggaatgagatgatgtcacttcaatctggaaattttttgcagcagtcttctcattcacaggcccaactttttcatcctcaaaatcctattgccgatgctcagaacctttcccaggaaactcaaggttctctctttcatagtccaaatcctattgtccacagtcagacttctacaacctcctctgaacaaatgcagcctccaatgtttcactctcaaagtaccattgctgtgttacagggctcttcagttcctcaagaccagcagtcaaccaacatatttctttcccagagtcccatgaataatcttcagactaacacagtagcccaagaagcattttttgcagcaccgaactcaatttctccacttcagtcaacatcaaacagtgaacaacaagctgctttccaacagcaagctccaatatcacacatccagactcctatgctttcccaagaacaggcacaacccccgcagcagggtttatttcagcctcaggtggccctgggctcccttccacctaatccaatgcctcaaagccaacaaggaaccatgttccagtcacagcactcaatagttgccatgcagagtaactctccatcccaggaacagcagcagcagcagcaacagcagcagcaacagcagcagcaacaacaacagagcattttattcagtaatcagaataccatggctacaatggcgtctccaaagcaaccaccaccaaacatgatattcaacccaaatcaaaatccaatggctaatcaggagcaacagaaccagtcaatttttcaccaacaaagtaacatggccccaatgaatcaagagcaacagcccatgcaatttcagagtcagtccacagtttcctcacttcagaacccaggtcctacccagtcggaatcatcacagacccccttgttccatagctctcctcagattcagttggtacaagggtcacctagttctcaagagcagcaagtaactctcttcttatctccagcatccatgtctgccttgcagaccagtataaatcaacaagatatgcaacagtctcctctttattcccctcagaacaacatgcctggaattcaaggagccacatcttcgcctcaaccacaggctactttatttcacaacacagcaggaggcacaatgaaccaactgcagaattctcctggctcatctcagcagacatcaggaatgttcttatttggcattcaaaataactgtagtcagcttttaacctctggaccagctacattgcctgatcagttgatggccataagtcagccaggccaaccacaaaacgagggccagccacctgtgacaacacttctttctcagcaaatgccagagaattctccactggcatcctctataaacaccaaccagaacatcgaaaagattgatttgcttgtttcattgcaaaaccaagggaacaacttgactggctccttttaactggatatgtaagtattgcattttggcttcttattgaaaagcatcagattttattcttcttaacagatttaggttaagtaatagtagttctaatcttttaaaatgtggctttctcacaagatgatacttttttactctgaataaaattattctgaatgatagcttatggtcatatagttaatacagaccaggccgggcgtggtggctcatgcctgtaatcccagcactttgagagaccaaggtgggcggatcacgaggtcaggcgatcgagaccatcctggctaacatggtgaaaccccgtctctactaaaaaatacaaaaaaaaaaaaa

SEQ ID NO: 3 - protein, 1484 aa, Homo sapiens
MSQTSGGEAGSPPPAVVAADASSAPSSSSMGGACSSFTTSSSPTIYSTSVTDSKAMQVESCSSAVGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAAAGALNVNVKKEISSPARPCSFEEAMKAMKTTGCNLDKVNIIPNALMTPLIPSSMIKSEDVTPMEVTAEKRSSTIFKTTKSVGSTQQTLENISNIAGNGSFSSPSSSHLPSENEKQQQIQPKAYNPETLTTIQTQDISQPGTFPAVSASSQLPNSDALLQQATQFQTRETQSREILQSDGTVVNLSQLTEASQQQQQSPLQEQAQTLQQQISSNIFPSPNSVSQLQNTIQQLQAGSFTGSTASGSSGSVDLVQQVLEAQQQLSSVLFSAPDGNENVQEQLSADIFQQVSQIQSGVSPGMFSSTEPTVHTRPDNLLPGRAESVHPQSENTLSNQQQQQQQQQQVMESSAAMVMEMQQSICQAAAQIQSELFPSTASANGNLQQSPVYQQTSHMMSALSTNEDMQMQCELFSSPPAVSGNETSTTTTQQVATPGTTMFQTSSSGDGEETGTQAKQIQNSVFQTMVQMQHSGDNQPQVNLFSSTKSMMSVQNSGTQQQGNGLFQQGNEMMSLQSGNFLQQSSHSQAQLFHPQNPIADAQNLSQETQGSLFHSPNPIVHSQTSTTSSEQMQPPMFHSQSTIAVLQGSSVPQDQQSTNIFLSQSPMNNLQTNTVAQEAFFAAPNSISPLQSTSNSEQQAAFQQQAPISHIQTPMLSQEQAQPPQQGLFQPQVALGSLPPNPMPQSQQGTMFQSQHSIVAMQSNSPSQEQQQQQQQQQQQQQQQQQSILFSNQNTMATMASPKQPPPNMIFNPNQNPMANQEQQNQSIFHQQSNMAPMNQEQQPMQFQSQSTVSSLQNPGPTQSESSQTPLFHSSPQIQLVQGSPSSQEQQVTLFLSPASMSALQTSINQQDMQQSPLYSPQNNMPGIQGATSSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 4 - DNA, 14177 bp, Homo sapiens
tgcaacggaaacttttggctccacgaacagaaaagcaagagcaaaaactcagttaaatgcgttcttcctccatctttgcctagtccaaaaggaaaaaaaaagaaacagaaaaaagaaaaactgtttggaagagtagcgttgaggtttgctttttacctgtttttcctctagaacagccaggtggattcacatgatccaattttttaattgtcttctatgtcccactccgaataacccggatgccccagcacaatcagagagatcgttttttttaaaaaaaatttcagcctccaaaggtaggaggggagtgttaggggagaaagtactttaattaaaaatcaataactcgaggtttttgggatacggtttgccatttcctaattaagaaatgggtttgacagtccctttgtacacactgctatgcaaaccccaaagggttggcggctgtccgggcgatgacactccggtcccctgcgagaccccgggccagccaggcccgtccgccgccggcctctggggtccgtccccggctcgcgcagacctctcgcttctctcggctctgtctcctgcgctcagctctgctcggggccggccgcctcaggctcgccgccaccaggtcgttgcaaataccttttccctccccggggccccagcgcgcggccacctcccagcctccccccctcccaccctggcagcggggccctttcccggctcaggaacagcagcagcccgggccgcgccggcaggaagcgaggcccatgttgctgctgttccctggcgcgcctccccgccctccgggggccgccacggctcttccgcgctcccgggcacccccctccgcgcctgcgctgtgccccacgggggcggggctcagattcctgtcagcggcggcggcggtggcggcgaccgtcagttttcgctgaggagaaacacgaaacggaccctttggctctcccccttccccttccccgtcctgaacccctctcctggtcaccgagaatcagtccccgtggagttccccctccacctcgccatcgtttcctcggtcctcggcccagtggaagtcactaccctcgaggaggaggcagcggcagccgccctcgcgtcgccgcccccggttcggtgcccgcggtcccggagaggaggtgccgccgccaccgccgctccccccctcccgctgccctcgggccgggctgggtcgagctgcgatgccctcggacttcatctcattgctcagcgcggacctagacctggaatcgcccaagtccctctactcgcgagattctctgaagttacacccatcacagaattttcatagagctggactattggaagaatctgtctatgatcttctcccaaaggagttacagttacctccatctagagaaacatctgtagcatcaatgagtcagacaagcggtggtgaggcaggctcgcctcctccagctgttgttgctgctgatgcttcttcagctccctcctcttcctccatgggcggtgcttgcagctcctttaccacctcttccagccctaccatttattctacctcagtcaccgacagcaaggctatgcaagtggagagctgctcctcagccgtgggggtaagtaacagaggggtaagtgaaaagcagttaaccagtaacacagttcagcagcatccatcaacaccgaagaggcacacagtcttgtacatctcaccaccacctgaggacttgctggataacagtcggatgtcctgccaggatgaggggtgtggattggaatctgagcagagctgcagtatgtggatggaggattccccctccaacttcagtaacatgagcaccagttcctacaatgataacactgaggtacctcgtaaatcacgaaaacgaaatccaaagcagaggccgggggtcaaacgacgagattgtgaagaatctaatatggatatatttgatgccgacagtgccaaagcacctcactatgtgctttctcagcttaccacggacaacaaaggcaactcaaaagcgggaaatggaacattggaaaaccaaaaaggaactggagtaaagaagagccctatgttgtgtggacaatatcctgttaaaagtgagggaaaggagctgaagatagttgtacaacctgagacacagcaccgagctcggtacctgactgagggcagccgtggctcagtgaaagatagaacacagcaaggctttcctacagtaaagctggaaggccataatgaacctgtagtgttgcaagtgtttgtgggcaacgactctggacgagtgaaaccacatggattttatcaggcctgcagagtaactggacgaaatacaactccttgcaaagaagtggacattgaaggcactactgttatagaagtcggccttgatcctagcaacaacatgacactggcggtggactgcgtagggatattgaaattgaggaatgctgatgtcgaagccagaataggaattgctggttccaagaagaaaagcactcgtgccagattggtttttcgagttaatatcatgaggaaagatggctccactttgacactgcaaacaccctcttctccaattttgtgtactcagccagcaggagtgccagaaatcttaaagaaaagcttgcatagctgttcagtgaaaggagaagaagaagtgtttttaatcggcaagaactttctgaaaggaactaaagttattttccaagaaaatgtttctgatgaaaactcttggaagtcagaagctgaaattgatatggaactatttcatcagaatcatcttattgtgaaggttcctccctatcatgaccaacatataactttgcctgtgtcagtgggaatatatgtagtgacaaatgctggaagatctcatgatgttcaaccattcacttacactccagacccagcagcagctggtgctttgaatgtaaatgtgaagaaggaaatatctagtccagcaagaccttgctcttttgaagaggccatgaaagcaatgaaaactactggatgtaatttagataaggtaaatattatccctaatgccctgatgactccactcataccaagcagtatgattaagagtgaagatgttactccaatggaagtaacagcagaaaaaagatcttccactatttttaagactacaaagtctgttggatcaactcagcaaacattagaaaacatctcaaacatagcaggaaatggctctttttcatcaccatcatcttcccacctaccttctgaaaatgaaaaacagcagcagattcagcccaaggcatacaacccagagaccctgacaactattcaaacccaggacatctcacagcctggtacttttccagcagtttctgcttctagtcagctgcccaacagcgatgcactattgcagcaggctacacagtttcagacaagagaaactcagtctagagagatattacagtcagatggtacagtggttaatttgtcacaactgactgaggcatcacaacaacagcagcagtcaccactacaagaacaagcacagactttacagcagcagatttcatcaaatatttttccatcaccaaatagtgtgagtcagcttcagaatactattcagcagctgcaagcagggagtttcacaggcagtactgctagtggcagcagtggaagtgttgacttggtccaacaagttttagaggcacagcagcagttatcttcagttttattttctgctccagatggtaatgagaatgttcaagagcagcttagtgcagatatttttcaacaagtcagtcaaattcagagtggtgtaagccctggaatgttttcctcaacagagccaacagtccataccagaccagataatttattacctggaagagctgaaagtgttcatccacagtctgaaaacacgttatctaatcaacagcagcagcagcagcagcaacagcaagtgatggaatcttcagccgcaatggtgatggagatgcaacagagtatctgccaggcagctgcccagattcagtcagagttattcccttcaactgcttcagcaaatggaaaccttcagcaatcgccagtttaccagcagacttctcacatgatgagtgcattgtctaccaatgaggatatgcaaatgcagtgtgaattgttttcttctcctcctgcagtttctggaaatgaaacttctacaactaccacacagcaggttgcaacccctggcactaccatgtttcagacatcaagttcaggagatggagaagaaactggaacacaagcaaaacagattcagaacagtgtctttcagaccatggtccaaatgcaacatagtggggacaatcaacctcaagttaaccttttttcatccacaaaaagtatgatgagtgttcagaatagtggtacccaacaacaaggtaatggtttattccagcaagggaatgagatgatgtcacttcaatctggaaattttttgcagcagtcttctcattcacaggcccaactttttcatcctcaaaatcctattgccgatgctcagaacctttcccaggaaactcaaggttctctctttcatagtccaaatcctattgtccacagtcagacttctacaacctcctctgaacaaatgcagcctccaatgtttcactctcaaagtaccattgctgtgttacagggctcttcagttcctcaagaccagcagtcaaccaacatatttctttcccagagtcccatgaataatcttcagactaacacagtagcccaagaagcattttttgcagcaccgaactcaatttctccacttcagtcaacatcaaacagtgaacaacaagctgctttccaacagcaagctccaatatcacacatccagactcctatgctttcccaagaacaggcacaacccccgcagcagggtttatttcagcctcaggtggccctgggctcccttccacctaatccaatgcctcaaagccaacaaggaaccatgttccagtcacagcactcaatagttgccatgcagagtaactctccatcccaggaacagcagcagcagcagcaacagcagcagcaacagcagcagcaacaacaacagagcattttattcagtaatcagaataccatggctacaatggcgtctccaaagcaaccaccaccaaacatgatattcaacccaaatcaaaatccaatggctaatcaggagcaacagaaccagtcaatttttcaccaacaaagtaacatggccccaatgaatcaagagcaacagcccatgcaatttcagagtcagtccacagtttcctcacttcagaacccaggtcctacccagtcggaatcatcacagacccccttgttccatagctctcctcagattcagttggtacaagggtcacctagttctcaagagcagcaagtaactctcttcttatctccagcatccatgtctgccttgcagaccagtataaatcaacaagatatgcaacagtctcctctttattcccctcagaacaacatgcctggaattcaaggagccacatcttcgcctcaaccacaggctactttatttcacaacacagcaggaggcacaatgaaccaactgcagaattctcctggctcatctcagcagacatcaggaatgttcttatttggcattcaaaataactgtagtcagcttttaacctctggaccagctacattgcctgatcagttgatggccataagtcagccaggccaaccacaaaacgagggccagccacctgtgacaacacttctttctcagcaaatgccagagaattctccactggcatcctctataaacaccaaccagaacatcgaaaagattgatttgcttgtttcattgcaaaaccaagggaacaacttgactggctccttttaactggatataaattccacgaagaaaatcctgattccaagatgtcctgagatcttgtggttccatgagaattattactttaaaaacaaaacaaaatataaaaaactgtgtttgagtaaactgatagattttactctgactgcaaaagagcacacctatgctgcttgttgcagtaactaaccaccaatgttaacatcttcatattttatattcctaataacagtgatgactgagaatctatttgagtttccagctggcagaattaattgttattattttcctaggcgcaatttccttaaacgtacagtttaaattcaaggctggaccactcagttattattgctattagaaaataatatatcatgtttacttttgttcttcattattttctttcctgcattgttttagtcaagtaatggcttttgaaaaagtaaagttcaataataactaaggctgtgatttttttcaatataaaaggcacagctgttggccaaagtgaaggaatcttttttcagttttattggagaaactgaagggtaacattctaacaagtaaactgtatgtgcagataaaagtactcttgatttaacacaaaggcagatgatacacttataaaactgggaacagctggaatgcttcttgattttattttttcagagagttgttagttctctgggtttctactaaggggtttagccataactgtgcatagaaaaataattatctgtaaaaaatgaaggggataatatatgataaattatgttctgatatcctcctacagtagtttaaattgacagaaaaatttgaatgttttcttcttaacccagtcttaggctggtattccctttttatatatatctatattacttttcacctctttttcactttactttagagaactattaatatactactggcttcatgaccctgtagcatctttggccactttaatctagggtgacctagcaatcctgcagcacagggcagagagtactgtcttaggaattattaggagttgattcctgagaaacaacacatttttccccatgaacggtgctgttctgaagtcttcaaatttttccctctaataggaaacagtataaattttaattaaaaaaaaaaggcaaactaaaatttcttgaaatatcacttctccctgatctgcagtgagtataaattcacttgtcacctcagtgctttacagtttgaagtggtcacttacctgatggttcccacaagccttaggctttacagggttgtatcattgacttaaaatgaagaattaacttgtgttacatctataaagagcaaaataacacactccagaacttggcagttgtagcattagttatacagttttgggtgttcttgccacccgtgggatgcctgcttctcactaccacctgtgtctggacacatgcttatgtctcattttccttttggcatgtggaaagctgtcaatgcagtgtaaggccaacgtgtgtgtggcttctatgtgttgagataatgttttggtatccttgtccgtttcatttattttttaagtgtacaaaaaataacctgttaattgttgaaggctacttttctgttctttttttttttttttttctatcctgtacatttagttgaactgtgcggaattgtggtgttggttttgtttacacagccagatttttccttctttttgttttgtgatgatcttcctttgttctttgaatgtgctcttttgtctttttctcttttttctcatgttttcttccctccacctccacccctttctttctttctctctctgattgagaggcattgaattacgttttcagtagtacaggcttcttgccgatatgaagggaacttttcagaaagagacctactctgggtcatttaattttgaatacagttttcaatcgttcaagttttggatggtttatatctaatgtgtgtttcatttttttggaaagctatattttgtatttaggaaatggtatactattttgctatttgtactgagtgagtacattggcataaatatagaaatttatatatatacatatatataaactattcttttttgccacacatttttgtggtaaatttgtgagtttgtctgatgttctaccacaacgtggcgtctgataacagtgagggggggtggggtttgttatgtctttattgagtatttaagtatcttttgaaacaaatgacctgttcatctgtggccattccatcaggcagttagttccttgatgtcagtagtgggctaaaggcagcttactgtgtgtttgctggagctttcactcagccaagtgttagagtcaggaaacccattgaggcaatggcgtcaaatggtgtttcacaagaatgagccattcagtctttgctcactatatatttaatattttattattgttgttattgttattattaattggctttctgtattctatgccttttatttataaagacactaagaaaacccatgtttgtaattttaataacatttttcccatcttgtaatatccagagctactttataaattctctgaaccaaaagtattttcctcagtgtatctcttctcccccagcccctattgggaaaaattacccagtatagttcaggttatgaggaggatcagccacacaatccagtgcttcagtttgaaaatgtaaaattctaaccctaaagtagggttggttgaaatttcagacaaagcaaacccagcaggtataaaaagtagtataaatacaaatctgtaagttatttttgaattttctgaacttttttctaagagattacataggagactaaagaaatctatctgttcaagttctaattaggatgattgttaatactgcactgtggatgaagtggcgactggcttgtgtgctgacttctgtggtttagcaagaggtttattgttatcaaatgctaattggcaatgccaagtcactgggaccaattttctgttttataatatctaagtttagaacagaatatatacctgaactgtagtggtttgatcggatggagacagaaaacccgatttttattctcataaattttgtggttatttatacaagggctgtgctatgctaccatattcttgttcaataataataggtttgttgttttttttacattgttaaatgttccttacccctaaaggtcaatgttaagtacaacattctgaaaatacaatttggctacgaagagtattcatcttctttgaagctcagtggttgatatttgtgctaataatgcaatttcctgattactgttacaagttatagctacatatgggagagactcagtgagccagcaaaggccatagaaacaacaatttattaaatgtatttatggcagaaggacctaaataaactgtgagccaccttttcttctttatattgttacatttaagtgttcttgctttcagcaactcacattaatgcttggagcttatctctttctctctctctctctctctctctgtgtgtgtgtgtgtatgtgtgtgtgtgtgtgtgtgtgtttccttattgtcattccattatatatccacaccaacatgggtgacgataattcaaagtcatattttgcctctaagcttgatcatgttacctttatgattaaagtatcatgttatttagccaatgcaaatctgttttaaaacaaatagtttaaaaaaagaacaagtttttaagggctttattatagaagaagtattaatgaaggactttccttcctccctccctttcctcccctccctgcctcccttcttcccttccatctccccctcctccctgccttctttgtttctccttcccttattcctccctccctcctttctcccttccttcctttcttccattcatccttccttgccttttatttttattttttgtaatatcacatgtgctgtagtttggaattttattctagtgcatttcttgctcatcagaacctcagctaatctacctaggaaaaatagtatcaaaggaaatgagaaagttgtatctgagtccctccagaactaagataattctttttgaccatttaagcctttataaatgcgttttgaccatttaagcctttataaatgcttgttttaggaaagtgaatctgttagatgcatcaacaaataatgaccaggacaaaacgatttaataattaaagtctcaaatcaccatggttatacattttcaccagaaatagtaatcttacaatttttcatttttctgatgaagatttctgttccaatatctgtttcctaatagattttttaaattaattagctttcctctgctttatgaccacaggttttatccctaaccgagacagctgtcttatatctgcatgccttagactgtgtggagggactccatgaagaaagaccataggttagaaaaataactcatagtatataccctagtaagtgggttagtagaatctcataacatgtattaaaaagaggttttcttctctgcttgtttgtgtcactagagcaaaattgtagagataatgctcataatgcagtaaatatcagaataatatctacaatatcatttgtggatggtcccaggtcccagtgctctagttactttacttctttttttttttttgagatggagtcttgctctgtctctcaggctagagcagtgtgcgatctcagctcactgcagcctccacctcccaggttcaagcgattctcctgcctcagcctcccaagtagccaggattacaggcaccctccactaggcccggctaattttttttgtatttttttagtagagatggggttttgccatgttggccaggctggtttcgaactcctaacctccagtgatccacctgcctcggcgtcccaaagtgctaggattacaggcatgagccaccacatccggcctaattacttctttaatccccatttatttttatgccattctagcctcatttattaataaaattatgtttttactttctctttcaggaaattttttaaattaatattttatatctagatctaatgctatggaaaagtgcctttttatcatttataatttcatttttcactatttccaaaaacacataaacaaatagtttcagtaggtcccagcttttactttttccatttaaaccttcttttctccatttcttccctttggcttaagaataaaagaaaaggtacattgctagaattgtttctttgggagagggtaaaagattacagaattagactgttcagcctttatataaactaaatttgtcttcatctcaaccagctaatggtaggtcttatctgaatactcatgagaattttagcatctgtgaaactccatgcaccagatgtgtgtaaatttcaggaagaaagtgttgaaagcattttctctgatgttaattagatggaaataaatcactaaaacatagtttaggtaaagcctgattatgccacttttttttaactagacagggcaaagttgtttatgttagtgtacttcttgtctatcctcagttaatttacctagacaaaaagtgtcaaaggaaatgagaaaaaggttatatctgactccctccagacctaagataattccttttgatcagatacagtcagatggagtgccttggtttttgttaattttgcctctattccagctccttaccacagcggtggtgcttaaagaaaggatcatcagcaacaggtcaggatagttctacctttgggatagggctgctttccccgtgctagtatttctgtgactgttagtggcactgaggactgcaaacttttatgcaatattcttaataccctattgatattatgcactttaatcattccaaagaagccaagaatgctgtatagtgatgattccttcctaatgaattcatcttaactatttagaatgttatgtcccttttcttttggatagccaacttggtataaatgttatatggatttttctaaaatgactatataggacttaagactttgaaatgtaatttacttataaggggaaataattatgctttagcacatcattttagaaacgtcacattttagaaacattcagcttgctaacctacatgtttgggaattcattaaaaccagttgtctatatattttgtgccatgtatataagaacattacaatatatctttttctacatatgtagtatgtgcaaccagtggttctcagagtatggttctcagcccaccagctagtatcagtatcacctgggaactagttagaaatgtaaattctttggccccatcccagacatactgagtcagaaactctggaatagggcccccgcaatctgttttcacaagccctccaggtgattctgatgcacactttaaagtttaggaaccactgggctaagactctgttgagatatagagtttttcttccactcagactgatatagttatacattgttcttcatgtaaattcagcttaacctggttatctataatcttttattggcaaaagttaattctcagtactgcctatagagatacagtgtattttatgtacatacacaattagtctaattcttgataattcagttaatttagtttggcattttcctaccacttactaaaaggtttacattaaatgactgatttaaatatataggtgcaatgttctatgtttattttaattgttatgacatttaagtagctaatataattgaccggtgctaaagtctcctgtttatccataaaatgggtacattatgggcagtgtaatacaagctttcttttcattgcctagtactttaccagcagaccacagttttgccctggctagaccaaccctcagaacaaaatcatcattccttgtatttatatttgtatctgagatagtaaacaagatggctggccaggtcaacatggcaccttaacttatttttttaataggtaaaacttcttcaaaagtagcttgctttgtataagaactaagctatcagtatagatatagctatccttggagcttatgtttcagacaagaattatttactaaaataaataataaacaagataatgcattatacaatttgggcatttctcgtttctcaagtgtatgcatcatggtaaatataaactaaccacaagataggtagattgattcatttcattttaatctccttgtgtaattcagtacctccataattgttctaatcttcttcccactgtttacaaattaccagttaattaactcgtgaaagaaaaattcacatatcagaataaaaataaatgtatactcactttataaaaatcaccactgctgtctttccttaatactagcagtggaaatgtaagtggcttactctacaaattttggtgctggcaaatacataggcaaactgttgggagctgctctagttacattcctcccttcttattccctttttctcttcctcactttattgcataacatattcctgtacccaaagcattctaccacagttctatttgactcccacttgtaataactcctttaaaaaattccatgtttaaccatatgaccctgcttgcttactcatattctccctccctctccccttcctttctctctcttccagaagtcatttgcctggtttgaaatattttgtagggattgcttattatattattttagctgatgaacctcaggacaacgtctacacacacacacatacatacacgcacacaaaatctcagctgttgaagagtgggcttggaatcagacttctgtgtccagtaaaaaactcctgcactgaagtcattgtgacttgagtagttacagactgattccagtgaacttgatctaatttcttttgatctaatgaatgtgtctgcttaccttgtttccttttaattgataagctccaagtagttgctaattttttgacaactttaaatgagtttcattcacttcttttacttaatgttttaagtatagtaccaataatttcattaacctgttctcaagtggtttagctaccattctgccatttttaatttttatttaattttatttgcttgagcacactgatcaaccactgaactgccttcttccattgtcctgcaatgatataagggttacatttttgtgtatatggctttcatagttgggatttcagagcactgataccagatattttcagtttgttctctgggggaatttcatttgcatctatgtttttagctatctgtgataacttgttaaatattaaaaagatattttgcttctattggaacatttgtatactcgcaactatatttctgtaaacagctgcagtcaaaaataaaacactgaaagttaaaaaaaaaaaaaaaaaaaa

SEQ ID NO: 5 - protein, 1531 aa, Homo sapiens
MPSDFISLLSADLDLESPKSLYSRESVYDLLPKELQLPPSRETSVASMSQTSGGEAGSPPPAVVAADASSAPSSSSMGGACSSFTTSSSPTIYSTSVTDSKAMQVESCSSAVGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAAAGALNVNVKKEISSPARPCSFEEAMKAMKTTGCNLDKVNIIPNALMTPLIPSSMIKSEDVTPMEVTAEKRSSTIFKTTKSVGSTQQTLENISNIAGNGSFSSPSSSHLPSENEKQQQIQPKAYNPETLTTIQTQDISQPGTFPAVSASSQLPNSDALLQQATQFQTRETQSREILQSDGTVVNLSQLTEASQQQQQSPLQEQAQTLQQQISSNIFPSPNSVSQLQNTIQQLQAGSFTGSTASGSSGSVDLVQQVLEAQQQLSSVLFSAPDGNENVQEQLSADIFQQVSQIQSGVSPGMFSSTEPTVHTRPDNLLPGRAESVHPQSENTLSNQQQQQQQQQQVMESSAAMVMEMQQSICQAAAQIQSELFPSTASANGNLQQSPVYQQTSHMMSALSTNEDMQMQCELFSSPPAVSGNETSTTTTQQVATPGTTMFQTSSSGDGEETGTQAKQIQNSVFQTMVQMQHSGDNQPQVNLFSSTKSMMSVQNSGTQQQGNGLFQQGNEMMSLQSGNFLQQSSHSQAQLFHPQNPIADAQNLSQETQGSLFHSPNPIVHSQTSTTSSEQMQPPMFHSQSTIAVLQGSSVPQDQQSTNIFLSQSPMNNLQTNTVAQEAFFAAPNSISPLQSTSNSEQQAAFQQQAPISHIQTPMLSQEQAQPPQQGLFQPQVALGSLPPNPMPQSQQGTMFQSQHSIVAMQSNSPSQEQQQQQQQQQQQQQQQQQSILFSNQNTMATMASPKQPPPNMIFNPNQNPMANQEQQNQSIFHQQSNMAPMNQEQQPMQFQSQSTVSSLQNPGPTQSESSQTPLFHSSPQIQLVQGSPSSQEQQVTLFLSPASMSALQTSINQQDMQQSPLYSPQNNMPGIQGATSSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 6 - DNA, 14123 bp, Homo sapiens
tgcaacggaaacttttggctccacgaacagaaaagcaagagcaaaaactcagttaaatgcgttcttcctccatctttgcctagtccaaaaggaaaaaaaaagaaacagaaaaaagaaaaactgtttggaagagtagcgttgaggtttgctttttacctgtttttcctctagaacagccaggtggattcacatgatccaattttttaattgtcttctatgtcccactccgaataacccggatgccccagcacaatcagagagatcgttttttttaaaaaaaatttcagcctccaaaggtaggaggggagtgttaggggagaaagtactttaattaaaaatcaataactcgaggtttttgggatacggtttgccatttcctaattaagaaatgggtttgacagtccctttgtacacactgctatgcaaaccccaaagggttggcggctgtccgggcgatgacactccggtcccctgcgagaccccgggccagccaggcccgtccgccgccggcctctggggtccgtccccggctcgcgcagacctctcgcttctctcggctctgtctcctgcgctcagctctgctcggggccggccgcctcaggctcgccgccaccaggtcgttgcaaataccttttccctccccggggccccagcgcgcggccacctcccagcctccccccctcccaccctggcagcggggccctttcccggctcaggaacagcagcagcccgggccgcgccggcaggaagcgaggcccatgttgctgctgttccctggcgcgcctccccgccctccgggggccgccacggctcttccgcgctcccgggcacccccctccgcgcctgcgctgtgccccacgggggcggggctcagattcctgtcagcggcggcggcggtggcggcgaccgtcagttttcgctgaggagaaacacgaaacggaccctttggctctcccccttccccttccccgtcctgaacccctctcctggtcaccgagaatcagtccccgtggagttccccctccacctcgccatcgtttcctcggtcctcggcccagtggaagtcactaccctcgaggaggaggcagcggcagccgccctcgcgtcgccgcccccggttcggtgcccgcggtcccggagaggaggtgccgccgccaccgccgctccccccctcccgctgccctcgggccgggctgggtcgagctgcgatgccctcggacttcatctcattgctcagcgcggacctagacctggaatcgcccaagtccctctactcgcgagaatctgtctatgatcttctcccaaaggagttacagttacctccatctagagaaacatctgtagcatcaatgagtcagacaagcggtggtgaggcaggctcgcctcctccagctgttgttgctgctgatgcttcttcagctccctcctcttcctccatgggcggtgcttgcagctcctttaccacctcttccagccctaccatttattctacctcagtcaccgacagcaaggctatgcaagtggagagctgctcctcagccgtgggggtaagtaacagaggggtaagtgaaaagcagttaaccagtaacacagttcagcagcatccatcaacaccgaagaggcacacagtcttgtacatctcaccaccacctgaggacttgctggataacagtcggatgtcctgccaggatgaggggtgtggattggaatctgagcagagctgcagtatgtggatggaggattccccctccaacttcagtaacatgagcaccagttcctacaatgataacactgaggtacctcgtaaatcacgaaaacgaaatccaaagcagaggccgggggtcaaacgacgagattgtgaagaatctaatatggatatatttgatgccgacagtgccaaagcacctcactatgtgctttctcagcttaccacggacaacaaaggcaactcaaaagcgggaaatggaacattggaaaaccaaaaaggaactggagtaaagaagagccctatgttgtgtggacaatatcctgttaaaagtgagggaaaggagctgaagatagttgtacaacctgagacacagcaccgagctcggtacctgactgagggcagccgtggctcagtgaaagatagaacacagcaaggctttcctacagtaaagctggaaggccataatgaacctgtagtgttgcaagtgtttgtgggcaacgactctggacgagtgaaaccacatggattttatcaggcctgcagagtaactggacgaaatacaactccttgcaaagaagtggacattgaaggcactactgttatagaagtcggccttgatcctagcaacaacatgacactggcggtggactgcgtagggatattgaaattgaggaatgctgatgtcgaagccagaataggaattgctggttccaagaagaaaagcactcgtgccagattggtttttcgagttaatatcatgaggaaagatggctccactttgacactgcaaacaccctcttctccaattttgtgtactcagccagcaggagtgccagaaatcttaaagaaaagcttgcatagctgttcagtgaaaggagaagaagaagtgtttttaatcggcaagaactttctgaaaggaactaaagttattttccaagaaaatgtttctgatgaaaactcttggaagtcagaagctgaaattgatatggaactatttcatcagaatcatcttattgtgaaggttcctccctatcatgaccaacatataactttgcctgtgtcagtgggaatatatgtagtgacaaatgctggaagatctcatgatgttcaaccattcacttacactccagacccagcagcagctggtgctttgaatgtaaatgtgaagaaggaaatatctagtccagcaagaccttgctcttttgaagaggccatgaaagcaatgaaaactactggatgtaatttagataaggtaaatattatccctaatgccctgatgactccactcataccaagcagtatgattaagagtgaagatgttactccaatggaagtaacagcagaaaaaagatcttccactatttttaagactacaaagtctgttggatcaactcagcaaacattagaaaacatctcaaacatagcaggaaatggctctttttcatcaccatcatcttcccacctaccttctgaaaatgaaaaacagcagcagattcagcccaaggcatacaacccagagaccctgacaactattcaaacccaggacatctcacagcctggtacttttccagcagtttctgcttctagtcagctgcccaacagcgatgcactattgcagcaggctacacagtttcagacaagagaaactcagtctagagagatattacagtcagatggtacagtggttaatttgtcacaactgactgaggcatcacaacaacagcagcagtcaccactacaagaacaagcacagactttacagcagcagatttcatcaaatatttttccatcaccaaatagtgtgagtcagcttcagaatactattcagcagctgcaagcagggagtttcacaggcagtactgctagtggcagcagtggaagtgttgacttggtccaacaagttttagaggcacagcagcagttatcttcagttttattttctgctccagatggtaatgagaatgttcaagagcagcttagtgcagatatttttcaacaagtcagtcaaattcagagtggtgtaagccctggaatgttttcctcaacagagccaacagtccataccagaccagataatttattacctggaagagctgaaagtgttcatccacagtctgaaaacacgttatctaatcaacagcagcagcagcagcagcaacagcaagtgatggaatcttcagccgcaatggtgatggagatgcaacagagtatctgccaggcagctgcccagattcagtcagagttattcccttcaactgcttcagcaaatggaaaccttcagcaatcgccagtttaccagcagacttctcacatgatgagtgcattgtctaccaatgaggatatgcaaatgcagtgtgaattgttttcttctcctcctgcagtttctggaaatgaaacttctacaactaccacacagcaggttgcaacccctggcactaccatgtttcagacatcaagttcaggagatggagaagaaactggaacacaagcaaaacagattcagaacagtgtctttcagaccatggtccaaatgcaacatagtggggacaatcaacctcaagttaaccttttttcatccacaaaaagtatgatgagtgttcagaatagtggtacccaacaacaaggtaatggtttattccagcaagggaatgagatgatgtcacttcaatctggaaattttttgcagcagtcttctcattcacaggcccaactttttcatcctcaaaatcctattgccgatgctcagaacctttcccaggaaactcaaggttctctctttcatagtccaaatcctattgtccacagtcagacttctacaacctcctctgaacaaatgcagcctccaatgtttcactctcaaagtaccattgctgtgttacagggctcttcagttcctcaagaccagcagtcaaccaacatatttctttcccagagtcccatgaataatcttcagactaacacagtagcccaagaagcattttttgcagcaccgaactcaatttctccacttcagtcaacatcaaacagtgaacaacaagctgctttccaacagcaagctccaatatcacacatccagactcctatgctttcccaagaacaggcacaacccccgcagcagggtttatttcagcctcaggtggccctgggctcccttccacctaatccaatgcctcaaagccaacaaggaaccatgttccagtcacagcactcaatagttgccatgcagagtaactctccatcccaggaacagcagcagcagcagcaacagcagcagcaacagcagcagcaacaacaacagagcattttattcagtaatcagaataccatggctacaatggcgtctccaaagcaaccaccaccaaacatgatattcaacccaaatcaaaatccaatggctaatcaggagcaacagaaccagtcaatttttcaccaacaaagtaacatggccccaatgaatcaagagcaacagcccatgcaatttcagagtcagtccacagtttcctcacttcagaacccaggtcctacccagtcggaatcatcacagacccccttgttccatagctctcctcagattcagttggtacaagggtcacctagttctcaagagcagcaagtaactctcttcttatctccagcatccatgtctgccttgcagaccagtataaatcaacaagatatgcaacagtctcctctttattcccctcagaacaacatgcctggaattcaaggagccacatcttcgcctcaaccacaggctactttatttcacaacacagcaggaggcacaatgaaccaactgcagaattctcctggctcatctcagcagacatcaggaatgttcttatttggcattcaaaataactgtagtcagcttttaacctctggaccagctacattgcctgatcagttgatggccataagtcagccaggccaaccacaaaacgagggccagccacctgtgacaacacttctttctcagcaaatgccagagaattctccactggcatcctctataaacaccaaccagaacatcgaaaagattgatttgcttgtttcattgcaaaaccaagggaacaacttgactggctccttttaactggatataaattccacgaagaaaatcctgattccaagatgtcctgagatcttgtggttccatgagaattattactttaaaaacaaaacaaaatataaaaaactgtgtttgagtaaactgatagattttactctgactgcaaaagagcacacctatgctgcttgttgcagtaactaaccaccaatgttaacatcttcatattttatattcctaataacagtgatgactgagaatctatttgagtttccagctggcagaattaattgttattattttcctaggcgcaatttccttaaacgtacagtttaaattcaaggctggaccactcagttattattgctattagaaaataatatatcatgtttacttttgttcttcattattttctttcctgcattgttttagtcaagtaatggcttttgaaaaagtaaagttcaataataactaaggctgtgatttttttcaatataaaaggcacagctgttggccaaagtgaaggaatcttttttcagttttattggagaaactgaagggtaacattctaacaagtaaactgtatgtgcagataaaagtactcttgatttaacacaaaggcagatgatacacttataaaactgggaacagctggaatgcttcttgattttattttttcagagagttgttagttctctgggtttctactaaggggtttagccataactgtgcatagaaaaataattatctgtaaaaaatgaaggggataatatatgataaattatgttctgatatcctcctacagtagtttaaattgacagaaaaatttgaatgttttcttcttaacccagtcttaggctggtattccctttttatatatatctatattacttttcacctctttttcactttactttagagaactattaatatactactggcttcatgaccctgtagcatctttggccactttaatctagggtgacctagcaatcctgcagcacagggcagagagtactgtcttaggaattattaggagttgattcctgagaaacaacacatttttccccatgaacggtgctgttctgaagtcttcaaatttttccctctaataggaaacagtataaattttaattaaaaaaaaaaggcaaactaaaatttcttgaaatatcacttctccctgatctgcagtgagtataaattcacttgtcacctcagtgctttacagtttgaagtggtcacttacctgatggttcccacaagccttaggctttacagggttgtatcattgacttaaaatgaagaattaacttgtgttacatctataaagagcaaaataacacactccagaacttggcagttgtagcattagttatacagttttgggtgttcttgccacccgtgggatgcctgcttctcactaccacctgtgtctggacacatgcttatgtctcattttccttttggcatgtggaaagctgtcaatgcagtgtaaggccaacgtgtgtgtggcttctatgtgttgagataatgttttggtatccttgtccgtttcatttattttttaagtgtacaaaaaataacctgttaattgttgaaggctacttttctgttctttttttttttttttttctatcctgtacatttagttgaactgtgcggaattgtggtgttggttttgtttacacagccagatttttccttctttttgttttgtgatgatcttcctttgttctttgaatgtgctcttttgtctttttctcttttttctcatgttttcttccctccacctccacccctttctttctttctctctctgattgagaggcattgaattacgttttcagtagtacaggcttcttgccgatatgaagggaacttttcagaaagagacctactctgggtcatttaattttgaatacagttttcaatcgttcaagttttggatggtttatatctaatgtgtgtttcatttttttggaaagctatattttgtatttaggaaatggtatactattttgctatttgtactgagtgagtacattggcataaatatagaaatttatatatatacatatatataaactattcttttttgccacacatttttgtggtaaatttgtgagtttgtctgatgttctaccacaacgtggcgtctgataacagtgagggggggtggggtttgttatgtctttattgagtatttaagtatcttttgaaacaaatgacctgttcatctgtggccattccatcaggcagttagttccttgatgtcagtagtgggctaaaggcagcttactgtgtgtttgctggagctttcactcagccaagtgttagagtcaggaaacccattgaggcaatggcgtcaaatggtgtttcacaagaatgagccattcagtctttgctcactatatatttaatattttattattgttgttattgttattattaattggctttctgtattctatgccttttatttataaagacactaagaaaacccatgtttgtaattttaataacatttttcccatcttgtaatatccagagctactttataaattctctgaaccaaaagtattttcctcagtgtatctcttctcccccagcccctattgggaaaaattacccagtatagttcaggttatgaggaggatcagccacacaatccagtgcttcagtttgaaaatgtaaaattctaaccctaaagtagggttggttgaaatttcagacaaagcaaacccagcaggtataaaaagtagtataaatacaaatctgtaagttatttttgaattttctgaacttttttctaagagattacataggagactaaagaaatctatctgttcaagttctaattaggatgattgttaatactgcactgtggatgaagtggcgactggcttgtgtgctgacttctgtggtttagcaagaggtttattgttatcaaatgctaattggcaatgccaagtcactgggaccaattttctgttttataatatctaagtttagaacagaatatatacctgaactgtagtggtttgatcggatggagacagaaaacccgatttttattctcataaattttgtggttatttatacaagggctgtgctatgctaccatattcttgttcaataataataggtttgttgttttttttacattgttaaatgttccttacccctaaaggtcaatgttaagtacaacattctgaaaatacaatttggctacgaagagtattcatcttctttgaagctcagtggttgatatttgtgctaataatgcaatttcctgattactgttacaagttatagctacatatgggagagactcagtgagccagcaaaggccatagaaacaacaatttattaaatgtatttatggcagaaggacctaaataaactgtgagccaccttttcttctttatattgttacatttaagtgttcttgctttcagcaactcacattaatgcttggagcttatctctttctctctctctctctctctctctgtgtgtgtgtgtgtatgtgtgtgtgtgtgtgtgtgtgtttccttattgtcattccattatatatccacaccaacatgggtgacgataattcaaagtcatattttgcctctaagcttgatcatgttacctttatgattaaagtatcatgttatttagccaatgcaaatctgttttaaaacaaatagtttaaaaaaagaacaagtttttaagggctttattatagaagaagtattaatgaaggactttccttcctccctccctttcctcccctccctgcctcccttcttcccttccatctccccctcctccctgccttctttgtttctccttcccttattcctccctccctcctttctcccttccttcctttcttccattcatccttccttgccttttatttttattttttgtaatatcacatgtgctgtagtttggaattttattctagtgcatttcttgctcatcagaacctcagctaatctacctaggaaaaatagtatcaaaggaaatgagaaagttgtatctgagtccctccagaactaagataattctttttgaccatttaagcctttataaatgcgttttgaccatttaagcctttataaatgcttgttttaggaaagtgaatctgttagatgcatcaacaaataatgaccaggacaaaacgatttaataattaaagtctcaaatcaccatggttatacattttcaccagaaatagtaatcttacaatttttcatttttctgatgaagatttctgttccaatatctgtttcctaatagattttttaaattaattagctttcctctgctttatgaccacaggttttatccctaaccgagacagctgtcttatatctgcatgccttagactgtgtggagggactccatgaagaaagaccataggttagaaaaataactcatagtatataccctagtaagtgggttagtagaatctcataacatgtattaaaaagaggttttcttctctgcttgtttgtgtcactagagcaaaattgtagagataatgctcataatgcagtaaatatcagaataatatctacaatatcatttgtggatggtcccaggtcccagtgctctagttactttacttctttttttttttttgagatggagtcttgctctgtctctcaggctagagcagtgtgcgatctcagctcactgcagcctccacctcccaggttcaagcgattctcctgcctcagcctcccaagtagccaggattacaggcaccctccactaggcccggctaattttttttgtatttttttagtagagatggggttttgccatgttggccaggctggtttcgaactcctaacctccagtgatccacctgcctcggcgtcccaaagtgctaggattacaggcatgagccaccacatccggcctaattacttctttaatccccatttatttttatgccattctagcctcatttattaataaaattatgtttttactttctctttcaggaaattttttaaattaatattttatatctagatctaatgctatggaaaagtgcctttttatcatttataatttcatttttcactatttccaaaaacacataaacaaatagtttcagtaggtcccagcttttactttttccatttaaaccttcttttctccatttcttccctttggcttaagaataaaagaaaaggtacattgctagaattgtttctttgggagagggtaaaagattacagaattagactgttcagcctttatataaactaaatttgtcttcatctcaaccagctaatggtaggtcttatctgaatactcatgagaattttagcatctgtgaaactccatgcaccagatgtgtgtaaatttcaggaagaaagtgttgaaagcattttctctgatgttaattagatggaaataaatcactaaaacatagtttaggtaaagcctgattatgccacttttttttaactagacagggcaaagttgtttatgttagtgtacttcttgtctatcctcagttaatttacctagacaaaaagtgtcaaaggaaatgagaaaaaggttatatctgactccctccagacctaagataattccttttgatcagatacagtcagatggagtgccttggtttttgttaattttgcctctattccagctccttaccacagcggtggtgcttaaagaaaggatcatcagcaacaggtcaggatagttctacctttgggatagggctgctttccccgtgctagtatttctgtgactgttagtggcactgaggactgcaaacttttatgcaatattcttaataccctattgatattatgcactttaatcattccaaagaagccaagaatgctgtatagtgatgattccttcctaatgaattcatcttaactatttagaatgttatgtcccttttcttttggatagccaacttggtataaatgttatatggatttttctaaaatgactatataggacttaagactttgaaatgtaatttacttataaggggaaataattatgctttagcacatcattttagaaacgtcacattttagaaacattcagcttgctaacctacatgtttgggaattcattaaaaccagttgtctatatattttgtgccatgtatataagaacattacaatatatctttttctacatatgtagtatgtgcaaccagtggttctcagagtatggttctcagcccaccagctagtatcagtatcacctgggaactagttagaaatgtaaattctttggccccatcccagacatactgagtcagaaactctggaatagggcccccgcaatctgttttcacaagccctccaggtgattctgatgcacactttaaagtttaggaaccactgggctaagactctgttgagatatagagtttttcttccactcagactgatatagttatacattgttcttcatgtaaattcagcttaacctggttatctataatcttttattggcaaaagttaattctcagtactgcctatagagatacagtgtattttatgtacatacacaattagtctaattcttgataattcagttaatttagtttggcattttcctaccacttactaaaaggtttacattaaatgactgatttaaatatataggtgcaatgttctatgtttattttaattgttatgacatttaagtagctaatataattgaccggtgctaaagtctcctgtttatccataaaatgggtacattatgggcagtgtaatacaagctttcttttcattgcctagtactttaccagcagaccacagttttgccctggctagaccaaccctcagaacaaaatcatcattccttgtatttatatttgtatctgagatagtaaacaagatggctggccaggtcaacatggcaccttaacttatttttttaataggtaaaacttcttcaaaagtagcttgctttgtataagaactaagctatcagtatagatatagctatccttggagcttatgtttcagacaagaattatttactaaaataaataataaacaagataatgcattatacaatttgggcatttctcgtttctcaagtgtatgcatcatggtaaatataaactaaccacaagataggtagattgattcatttcattttaatctccttgtgtaattcagtacctccataattgttctaatcttcttcccactgtttacaaattaccagttaattaactcgtgaaagaaaaattcacatatcagaataaaaataaatgtatactcactttataaaaatcaccactgctgtctttccttaatactagcagtggaaatgtaagtggcttactctacaaattttggtgctggcaaatacataggcaaactgttgggagctgctctagttacattcctcccttcttattccctttttctcttcctcactttattgcataacatattcctgtacccaaagcattctaccacagttctatttgactcccacttgtaataactcctttaaaaaattccatgtttaaccatatgaccctgcttgcttactcatattctccctccctctccccttcctttctctctcttccagaagtcatttgcctggtttgaaatattttgtagggattgcttattatattattttagctgatgaacctcaggacaacgtctacacacacacacatacatacacgcacacaaaatctcagctgttgaagagtgggcttggaatcagacttctgtgtccagtaaaaaactcctgcactgaagtcattgtgacttgagtagttacagactgattccagtgaacttgatctaatttcttttgatctaatgaatgtgtctgcttaccttgtttccttttaattgataagctccaagtagttgctaattttttgacaactttaaatgagtttcattcacttcttttacttaatgttttaagtatagtaccaataatttcattaacctgttctcaagtggtttagctaccattctgccatttttaatttttatttaattttatttgcttgagcacactgatcaaccactgaactgccttcttccattgtcctgcaatgatataagggttacatttttgtgtatatggctttcatagttgggatttcagagcactgataccagatattttcagtttgttctctgggggaatttcatttgcatctatgtttttagctatctgtgataacttgttaaatattaaaaagatattttgcttctattggaacatttgtatactcgcaactatatttctgtaaacagctgcagtcaaaaataaaacactgaaagttaaaaaaaaaaaaaaaaaaaa

SEQ ID NO: 7 - protein, 1458 aa, Mus musculus
MGGACSSFTTSSSPTIYSTSVTDSKAMQVESCSSAVGVSNRGVSEKQLTGNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADGAKAPHYVLSQLTTDNKGNTKAGNGTLDSQKGTGVKKSPMLCGQYPVKSEGRELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAEDCVGILKLRNADVEARIGIAGSKKKSTRARLVFRVNITRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTNTPDPAAGALNVNVKKEISSPARPCSLEEAMKAMKTTGCNVDKVTILPNALITPLISSSMIKTEDVTPMEVTSEKRSSPIFQTTKSIGSTQQTLETISNIAGGAPFSSPSSSSHLTPESENQQQLQPKAYSPETLTTIQAQDISQPGTFPAVSAASQLPSSDALLQQATQFQTREAQSRDTIQSDTVVNLSQLTEASQQQQSPLQEQAQTLQQQIPSNIFPSPSSVSQLQSTIQQLQAGSFTGSAAGGRSGSVDLVQQVLEAQQQLSTVLFSTPDGNENVQEQLNADIFQVSQIQNSVSPGMFSSAESAVHTRPDNLLPGRADSVHQQTENTLSNQQQQQQQQQQVMESSAAMVMEMQQSICQAAAQIQSELFPSAASASGSLQQSPVYQQPSHMMSALPTNEDMQMQCELFSSPPAASGNETSTTTTPQVATPGSTMFQTPSSGDGEETGAQAKQIQNSVFQTMVQMQRSGDSQPQVNLFSSTKNIMSVQNNGTQQQGNSLFQQGSEVMSLQSGNFLQQSSHSQAQLFHPQNPIADAQNLSQETQGSIFHSPNPIVHSQTSTASSEQLQPSMFHSQNTIAVLQGSSVPQDQQSPNIFLSQSSINNLQTNTVAQEEQISFFAAQNSISPLQSTSNTEQQAAFQQQPPIPHIQTPILSQEQAQPSQQGLFQPQVALGSLPPNPMPQNQQGPIFQTQRPIVGMQSNSPSQEQQQQQQQQQQQQQQQQQQQQSILFSNQNAMATMASQKQPPPNMMFSPNQNPMASQEQQNQSIFHQQSNMAPMNQEQQPMQFQNQPTVSSLQNPGPTQSESPQTSLFHSSPQIQLVQGSPSSQDQQVTLFLSPASMSALQTSINQPDMQQSPLYSPQNNIPGIQGSTSSPQPQATLFHNTTGGTINQIQNSPGSSQQTSGMFLFGIQNNCSQLLTSGPATLPDQLMAINQQGQPQNEGQSSVTTLLSQQMPETSPLASSVNSSQNMEKIDLLVSLQSQGNNLTGSF

SEQ ID NO: 8 - DNA, 13040 bp, Mus musculus
ctagacctggaatcgcccaagtccctgtactcgcgagattctctgaagttacacccatcacagacttttcatagagctggactattggaagaatctgtctatgatcttctcccaaaggagttacagttacctccacctagagaaacatctgtagcatcaatgagtcagacaagcggtggtgaggcaggctcgcctcctccagctgcagttgctgctggatttgcctctgaagcagggagtgtctgcattaaaaatgacctgtagttctctgcttcatagatgcttcttcagctccctcctcttcctccatgggcggtgcttgcagctcctttaccacctcttccagccctaccatttattctacctcagtcaccgacagcaaggctatgcaagtggagagctgctcctcagccgtgggggtaagtaacagaggggtaagtgaaaagcagttaaccggtaacacagttcagcagcatccatcaaccccgaagaggcacacagttttgtacatctcaccaccacctgaggacttgctggataacagtcggatgtcctgccaggatgaggggtgtggattggaatctgagcagagctgcagtatgtggatggaggattccccctccaacttcagtaacatgagcaccagttcctacaatgataacactgaggtacctcgtaaatcacgaaaacgaaatccaaagcagaggccgggggtcaaacgacgagattgtgaagagtctaatatggatatatttgatgccgacggtgccaaagcacctcactatgtgctttctcagcttaccacggacaacaaaggcaacacaaaagctggaaatggaacattggacagccaaaagggaactggagtaaagaagagccctatgttgtgcggacagtatccggttaaaagtgaggggagggagctgaagatagtggtacagcctgaaacccaacaccgagcccggtacctgacagagggcagccgaggctctgtaaaagacagaacacagcaaggctttcctacggtgaagctggaaggccataatgaaccagtggtgttgcaggtatttgtgggcaatgattctggtcgagtaaaaccacatggattctatcaggcctgtagagtaactgggcgaaatacaactccctgcaaagaagtggacattgaaggtaccactgttatagaagttggtcttgatcctagcaacaacatgacactggcggaggactgtgttggaatattgaagctgaggaatgccgatgttgaagccagaattggaattgctggatctaagaaaaagagcactcgtgccagattggtttttcgggttaatatcacaaggaaagatggctctactctgacattgcagacaccttcttcccccattttatgcactcagccggcaggagttcctgagatcttaaagaaaagcttgcatagctgttcagtgaaaggagaggaagaagtatttttaattgggaaaaactttctgaaaggaactaaagttattttccaggaaaatgtttctgatgaaaactcttggaaatcagaagctgaaattgacatggagttattccatcagaaccatcttattgtgaaggttcctccgtatcatgaccaacatataactttgcctgtatcagtgggaatatatgttgtgaccaatgctggaagatctcatgatgttcagccatttacgaacactccggatccagcagctggtgctttgaatgtaaatgtgaaaaaggagatatctagtccagcaagaccttgctctcttgaagaagccatgaaagcaatgaaaacgactggatgtaacgtagataaggtgactatccttcctaatgccctgatcactccactcatatcaagcagtatgattaagactgaagatgttactccaatggaagtaacttcagaaaaaagatcttccccaatttttcagactacaaagagcattggatcaacccagcaaaccttagaaactatttctaacatagcggggggtgcacccttctcctcaccgtcgtcatcttcccacttaactcctgaaagtgagaatcagcagcagcttcagcccaaggcatacagcccagagaccctgacaactatccaggcacaggacatctcacagcccgggacctttcccgcagtttctgctgctagtcagctgcccagcagcgatgcactactgcagcaggcaacacagtttcagacaagagaagctcagtccagagacacaatacagtcagatacagtggttaacttgtcacagttaactgaagcatcacagcagcagcaatccccactacaagaacaagcacaaacgttacagcagcagataccatcaaatatttttccatcgccaagcagtgtcagccagctccagagtactattcagcagctgcaagcaggaagtttcacaggcagcgctgctggcggcaggagtggaagcgttgatttggtccagcaggttttagaggcacaacagcagttatcgactgttctgttttctactccagatgggaatgagaatgttcaagaacagcttaatgcagacattttccaagtcagtcaaattcaaaatagtgtaagccctggaatgttttcttcagcggagtctgcagttcacactagaccagataacttactacctgggagggctgacagcgtccatcaacagactgaaaatacactgtctaatcagcagcagcagcaacagcagcagcagcaagtgatggagtcatcagctgcaatggtgatggagatgcagcagagcatttgtcaagcagctgcccagatccagtcagagctgtttccttcagctgcttcagcaagtggaagccttcagcagtctccagtttaccagcagccttctcacatgatgagtgcactgcctaccaacgaggacatgcagatgcagtgtgagctgttctcgtctccccctgcagcttctggaaacgaaacttccacaaccaccacgccacaggtggcaacccctggctccaccatgtttcagacaccaagttcaggagatggagaagaaactggagcacaagcaaaacagattcagaacagcgtctttcagacaatggtccaaatgcagcgcagtggtgacagccaacctcaagtaaacctcttttcatctacaaaaaatataatgagtgttcagaataatggtacccagcaacaagggaatagcttgttccagcaagggagcgaggtgatgtcacttcagtctgggaactttttgcagcagtcctctcattcacaggctcagctctttcatcctcaaaatcctattgctgatgctcagaacctttcccaggaaactcaaggttcaatttttcatagtccaaatcctattgtccacagtcaaacttccacagcatcctctgaacaactgcagccttcaatgtttcactctcagaataccattgctgtgttacagggctcttcagttcctcaagaccagcagtcacccaacatatttctttcccaaagttctatcaataatcttcaaactaacacagtagcccaagaagagcagatttcattttttgcagcacagaactcaatttctccacttcagtcaacatcaaacactgagcagcaagctgctttccaacagcagcctccaatcccacacatccagacccctattctttcccaggaacaggcacaaccctctcagcaagggttatttcagcctcaggtggcccttggctccctgcctcctaatccaatgcctcaaaaccagcaaggtccaattttccaaacacagcgcccaatagttggcatgcagagtaactctccatcccaggagcagcagcagcagcaacagcagcaacagcagcagcagcaacagcaacaacaacagcaacagagcattttattcagtaatcagaatgccatggctacaatggcctcccagaagcagccaccaccaaacatgatgtttagcccaaaccagaacccaatggctagtcaggagcagcagaaccagtcaatttttcaccagcaaagtaatatggccccaatgaaccaagagcagcagcccatgcaatttcagaatcagcctacggtttcctcacttcagaacccaggtcctacgcagtctgagtcaccacagacctccctgttccatagctctcctcagatccagttggttcaagggtcacctagttctcaagatcagcaagtaacactcttcctctctccagcatccatgtctgcattgcaaaccagtataaaccaaccagacatgcagcagtctcctctttattccccgcagaacaacatccctggaatccaaggaagcacttcttcaccccagccacaggctactttatttcacaacactacaggaggcacaataaaccaaatacaaaattctcctggctcatctcagcagacttcaggaatgttcttatttggcattcaaaataattgtagtcagcttttaacctccggaccagctacattgccagatcagctgatggccataaatcagcagggccaaccacaaaatgagggccaatcttctgtgacaacacttctttctcagcaaatgccagagacttccccactggcctcctctgtgaacagcagtcagaacatggaaaagattgatttgcttgtttcattgcaaagccaagggaacaatttaaccggctccttttagtgagataaattccatgaagaattctgtaacagtgattccaagatatcctgagaactgtggttccataaagattattgaactctggtgaagaactatgtttgagtaaatggatagatttcctggctataagagtacacccatgctgcctgttgcagtgatttgccaccaatgtgctttttgtttctaataacagtgatgactgagagcctgttagattttccagctgacagcattgttgcttgtgtcctctgtttgtaactgcaaggctgggcctctcagttattattgctattaggaaatcatatttgtcatgtttacttatattcttacttcctttcctgcattattttaatcaagtagtggcttaggacaacgtaaagtccagtagtgactaaggctgtgcttttgttgatgtaaaaaacgtggctgttggccaaagtgaaggagtttcttttagttttcatggagaagctgaaggagtaatttagctgtattaggaactctgcagatctgaggactcctcctcacacacacaagcagatgacatgcctgcaaaactggttacagctgggaagttattgagttcatttttcagagagttgttagttctctgtatttctactaagggatttagccataactgcataaaaatattttgttctatgaaaatgaagggggtgatatatgatgtgttacgttctcatagtctcctccggtagcttaaactgacagtactttgagcctgttgtcttcttgcccagcgttaggctagtattcccttcttatgtgcatcacctgtattgtgttgtctgcattttttcttcgtagttttattaatattctactggctttctgaccctgtaggaattagagaaggcagccacattgatccagtgacctggcagtcagcagcacaggccaggaatactcttttaggagttattgggatttgattcctgggaaaaaaccattctctttttttcttccatgaacgggtgctgttctgaagtcttcaaaattctccctctgctgataaatagcagtatacattttaattttacaaacaaggcaaacaaacatttctcaaactattacttctccccacgctgcagtgactgtttacatgtcagtcagctcagtgctttacagttgaagtggtcacttacatgatggttcccacagccttgggctccacagggtcatagcattgatttaaaaggaagagttaactgtgttgcatctacagagagcaaaattacacactccagaacttgcatttgtagcattagttgtagagttttgggtgtccttaccacccgtgggatgcctgcttctcactaccacctacatctggacacgtgcttatgtctcatttatctttggcatgtgggaagtgtcaatgcagcgttagcccatcatgtatgcagcttctatatgttgacatgctttggtttcctgttcaattcttttactttctaagcttttaaaaattagccagttcatggttgttgcctttttccttacttttcctttcagtttttctccccctatcctccacatgtgttgaactgtgtggagcttggtgttggtttagtttatatggccagatcctgctttgggactcttcctttgttatttgactgtggttcttgtcatctgtttcttctctttgtccttctccctctgccctggcccttcctttccctctctgagaggcattggattacgttttcagtagtacaggcttcttgccaatatgaagggaactttccagaaagagacctactctggtcatttaattttgaaaacagttttcaatcgttcaagttttggatggtttatatctaatgtgtgtttcatttttttggaaagctatattttatatttaggaaatggtatactattttgctatttgtactaagtgagtacattggcataaatatagaaatttatatatatatatatatatacatatataaactattcttttttgccacacatttttgtggtaaatttgtgagtttgtctgatgttctaccacaacgtggcgtctgataacagtgaggggggtttgttatgtctttcttgagtatttaagcaccttttgaagcaaatggcctgtttatctgtagccattccatcaggcagttcctcgatgttattagggagctgaagggcgactgactgtgtgtttgctggatcttcactcagtgagatgttaaagcgcaaggaaattcatttgaggcagtgtgtcaaatggtgtttgacaagaatgagccactctgtctttgttcactatatatttaatattttattattgttgttgttgttgttattaattggctttctgtatcctatgccttttatttataaagacactaaaaaaaaaccatgtttgtaattttaataacatttccccatcttttaatatccagagctactttataaattctctaaaccaaaagtatttcctcaagatctcctttctccccccagttcctatagggaaaaatcacccaacatagttcgggttacaagaagggtcagccacacaattcagtgcttcagttttaaaagtaaaacagacagacactagagtgttgtccagagcatcatagaaagcagacctagcagatttaggaaaaacagaagtcaaagtttaataccacatttacttctgagttttctgaattactttgtgagatcatgtagaagactcaggaaatctgtttcttcaccaagatcaagttagaatgtttgacactgccatgctggggatgacgtggcagcaggaagcctggctcgtgtggtggcttctggtcgttctggactagcagcttatccttataaaatgctgattggcaatgccaagtcaccgggaccaatttctgttttataatgtttaagtttagaacagaacatcacctgaactgtagtgctttgagaggatggaggtggaaacccccattttcagtctcatcacttctgtggttatttatacaagggctgtgctgtgctaccatattcctgctcgaatatgatggagtttgtttattttctcgcattgttaaatgttccttgcccctaaaggtccatgttaagaacaatgttctgaatatatgatttggttacaaaaagtatgtcttcttattgaagagttagctcagtggttgatatttgtgctaagatgcagcttcccagttactgttacaagttacagctgccaatgggaagactcaataagccagcaaaggccttgaaaatcatgatttattaaatttatatctggcagaaggacctaaataaactgtgaactacattgtattcctttgttacatttggctcttgctttcagcaattcacagaaatgcttggagctctctctctctctctctctctctctctctctctctctctctctctctctgtctctcccctcccttcctctctccctttccctctccctctctgtccccctccctctgtccctccctctacatcctttccttcctctctccctttccctctccctttccctctctctctccctctctccctccctctctactctcccactacatccttccctttctctctccctctttccctcccttcctctttccatccacacacacctctgtgtgtgtgtgtgtgtgtgtgtgtgtgtgtgtgtgtgtgtgtatgtgtgtgtgtgtgtatgcaagttattccttcttgtcatttcacaccaaggagaagatattacatacttttttcctaagcattgctgtggacttttctgattctagcatcatgttgctatttggtatttagccattgtagacctgtcttacccttagaccaagtgttaaaagggcttttattaatagaagcagtattaacaagaggcctctttctttccctcccattctcccatcctttcttgccccctctttgtataatattgtgagttataatttataattgcattatagtagatttattgcctatcagaattttgactaatgtacccagaaaaaatagaaggaagaaattgtgcatctgtcccttcagaattaaagggacaattccttttgaccttttgagtgtttgtttttattttttcagcaaggttgcagtatcctgcccaagtcctgggcctgctggtatgaagcaccttaccgggaccccttttttaaaccagtataaatgcttgttttgggaaaatgaaatctgtcagcaaataagaaccaaaactgaataattctttttttttaattattattatttttgtattttttctgtacattgacactgtcttcagacaccagaggagggcattggatcccattacaaatggttttgagccagcaagtggttgctgggaattgaactcaggacctctgggaaagcaatcagttctcttaaccactgtgccatctcttcggctcctgaataattcttaaattcagcagagttatacttactcacaggggttagcctgagaatttttcattttcctagtaaaattttttgttatccctatttcctgattgttaattagttgaaagttatgggagaacttgtggctttactttgcatctctagcacaggctctatctctaaccaaaatgaccactttatatctgggtcctttgggttacctggaggggctcagtgaaagagaaaaactaattcaatagacatacgagaaaaataacccaggccttgaggagtggctcagaggtagagagtttatgtagcatgcgcaaggccctgaatttggtccccagcaccaccaaaagggagagagagatcttgggtacacgtgctagcaagaggattagcactcacccattcatcacacatactgagagctttcccattctgcctacttttgtcactagagctaaactgaaggtgacattcatgatgcaggagattgactctcttcagtatatgtaaattatcccaataccctaattactttctaactgtctgtctattttcatgccatgctaagcctcatattaataaagttctgtttttactgctcttgctcttcaggcaattttttacattaatattttatatgtagattaaatgttatggaaaagtgcctttttaccatgccagtgcccccttctttagtatatgtgctgccaaagcaagcaccgtgcccccttcttaaaaacttcaccttccactattgccaaaattctgtgaacaactggttttatcaggtcccaacttgtactttttccatgtgaccattattttctccatttcttggtcaccataggagataaccttgtcagttcctttgacttaaaaataatagagaaagtgctttggcagaattgcttgggaaagtaagtgcacactgttcaggcttgtgtgaatctaatttcctcatcccaacaaattatagatggctatcacctgaataagaacaaaaataaacatttggctgtctgtgagacttcatgcacaaaaatgtgtgttaaatgttataaaggcactactcaacagcctcttctcagatcctaattagctgggggtgggggggaacagtcactaaaacttaggttttgtaaggcctggaggtaacattgggtttttttccttaattaaatgtgacaaagtagtttatttcttatctatctgaacttcagttaattgactttagaaaacttaatgtcagaaaaaacttagaagtcatttgagtggggggggggggagagctaaagtaatgttttagtctcagatacagttgggttgagtgtcctggttttgtccattttgtttctgatccaactctttatggtggtgcttgaagaaagagacatcagcatcaaattcaggacagtagtaacttaaactccacaacaaaggtgtgttccccatgcaagtatgattgtaactgttggtggactgaagatagcatagttatgcagtatttgtaatactctattgtgcactttaatcattccaaaaaaagccaagaatacatcttaactattcagaatactgcatatttattttggatatatggcttggtgtgttaggttcctgcttaattaaagtaacttacaataagtttgaaattactattttctaaaggacttaagaccttgaaatgtagctgtagggaaatgactatattttgcagaccattttagaaatagtaaaatttgactggctgatgagtatgtcggagagctacttagaaccattccccttccttcgtgtcccttatataagaatgctatagtgtatagtttctacccctgtggtttatgcaaaccagtatggtcctctgcccagcagctagctgatgcagtatgagcagggaactagttaaaggtgcacattcttgggcagtagcccaggcatactgaattagatctgaagtggggcccagcagtctgtctaacccaagtccccagtgagtccagtgcatccttagtttcagagccactctaacggatgagttctatctagtgaaagccagagaatttcttcccacttttaagagataattatttcttctttgtgtatattcagccttacctggttatctgtgatcatttctcaacaataataatttattctcagtactgcctaagaaatgcatgtgttttatgtatatacagtgtagttattgataattcatttaacttagtttggcatttccctaccactgactaaaaagtttacattaactgactgatttcaatatataggtgcaatgttctgtatttctttaattgttgtgacacttacttagctaaaataattgatgggtgctacagtctcctgtttatgcataacatgggtacattgtgggcagtgtaatacaagctttctctttgttgcctcttaatttaccagcaggccatggttttgctcttggctagaccagctctcagaacagaattgccattccataccatttttcttgtatctgagatctcagatagatggctagccaggccaacatagcaccttatttaatttcttctcttaatagattcttttcaagtagcttacttggcatttgaacagtgcagtcaagtgatggttctccttggaactcacacttcaggtgaagtacttaacaaaatgataagacagtgcattttataatttgggtatcccctttctcagttgtgtgcatcttaataaatacaagctaagcaggtttatttttttcctttagaattcagcacttccatcatccttccactgtttacaaatcgctgatgagttaactcatgatagagagattcacttttcagaataaaactaactgaatacttgctttctgagaatcactgcttctgtaccacactagcagtgctaagtggcttactctacaaactggtgctggaaagtaagtaggcaagctgttgggatgctgtagagaccccctcccttccccttctgtcactggatacagagcattctacacaatttatttaaccaaggtttgtaacacctcctttaaagatcctgcattcaacccatgtcacccctacctgctctctcacattggtccttccctttcccaggaacccaccccagcagtaataatatgcatggtttgtaggggtggcctattgttactattttagctagtgagcctcaggaaagcttacactcacacataaccccagacgcttgcaagtgagttgagaatcaaacttctgtgacctggctggagttagagttcagcgacagagcctttgtacacacaaggcccaaggtaggtgtaatccctggtaccaaagagagacagacagacagacagagctgtcctgtatcgaagtgatcacaatttcactcattagagaccttctgcagtgcaattgaactaaccccttctctctaatgaatgtatctgttcaccttgttccctctaaggataagctccaagtgattgctgctttcctggcaacttgactgagtttcactctcttcttttacttcctgttttaatgagtaacttggaatatttcattaacatgttctcaagtgatttagctgccattctgccattttgagttttgtttaattttatctgcttgagcacactgatcaaccactgacctgccttcttgcattgtcctgcagtgacgtaagagtgacattgtgtatacggcttcatagttggaatttcagagcactgacaccagatattctcagtttgttctctgggggaatttcatttgcatctatgtttttagctatctgtgataacttgttaaatattaaaaagatattttgcttctattggaacatttgtatactcgcaattatatttctgtaaacagctgcagtcaaaaataaaacattgaaagttttcattttgca

SEQ ID NO: 9 - protein, 1487 aa, Mus musculus
MSQTSGGEAGSPPPAVVAADASSAPSSSSMGGACSSFTTSSSPTIYSTSVTDSKAMQVESCSSAVGVSNRGVSEKQLTGNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLDSQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNITRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAAGALNVNVKKEISSPARPCSFEEAMKAMKTTGCNVDKVTILPNALITPLISSSMIKTEDVTPMEVTSEKRSSPIFQTTKSIGSTQQTLETISNIAGGAPFSSPSSSSHLTPESENQQQLQPKAYNPETLTTIQTQDISQPGTFPAVSAASQLPSSDALLQQATQFQTREAQSRDTIQSDTVVNLSQLTEASQQQQSPLQEQAQTLQQQIPSNIFPSPSSVSQLQSTIQQLQAGSFTGSAAGGRSGSVDLVQQVLEAQQQLSSVLFSTPDGNENVQEQLNADIFQVSQIQNSVSPGMFSSAESAVHTRPDNLLPGRADSVHQQTENTLSNQQQQQQQQQQVMESSAAMVMEMQQSICQAAAQIQSELFPSAASASGSLQQSPVYQQPSHMMSALPTNEDMQMQCELFSSPPAASGNETSTTTTPQVATPGSTMFQTPSSGDGEETGAQAKQIQNSVFQTMVQMQRSGDSQPQVNLFSSTKNIMSVQNNGTQQQGNSLFQQGSEMMSLQSGNFLQQSSHSQAQLFHPQNPIADAQNLSQETQGSIFHSPNPIVHSQTSTASSEQLQPSMFHSQNTIAVLQGSSVPQDQQSPNIFLSQSSINNLQTNTVAQEEQISFFAAQNSISPLQSTSNTEQQAAFQQQPPISHIQTPILSQEQAQPSQQGLFQPQVALGSLPPNPMPQNQQGPIFQTQRPIVGMQSNSPSQEQQQQQQQQQQQQQQQQQQQQSILFSNQNAMATMASQKQPPPNMMFSPNQNPMASQEQQNQSIFHQQSNMAPMNQEQQPMQFQNQPTVSSLQNPGPTQSESPQTSLFHSSPQIQLVQGSPSSQDQQVTLFLSPASMSALQTSINQPDMQQSPLYSPQNNIPGIQGSTSSPQPQATLFHNTTGGTINQIQNSPGSSQQTSGMFLFGIQNNCSQLLTSGPATLPDQLMAINQQGQPQNEGQSSVTTLLSQQMPETSPLASSVNSSQNMEKIDLLVSLQSQGNNLTGSF

SEQ ID NO: 10 - DNA, 4653 bp, Mus musculus
gggagatgaagtccgagggcatcgcagctcgacctggaatcgcccaagtccctgtactcgcgagattctctgaagttacacccatcacagacttttcatagagctggactattggaagaatctgtctatgatcttctcccaaaggagttacagttacctccacctagagaaacatctgtagcatcaatgagtcagacaagcggtggtgaggcaggctcgcctcctccagctgtagttgctgctgatgcttcttcagctccctcctcttcctccatgggcggtgcttgcagctcctttaccacctcttccagccctaccatttattctacctcagtcaccgacagcaaggctatgcaagtggagagctgctcctcagccgtgggggtaagtaacagaggggtaagtgaaaagcagttaaccggtaacacagttcagcagcatccatcaaccccgaagaggcacacagttttgtacatctcaccaccacctgaggacttgctggataacagtcggatgtcctgccaggatgaggggtgtggattggaatctgagcagagctgcagtatgtggatggaggattccccctccaacttcagtaacatgagcaccagttcctacaatgataacactgaggtacctcgtaaatcacgaaaacgaaatccaaagcagaggccgggggtcaaacgacgagattgtgaagaatctaatatggatatatttgatgccgacagtgccaaagcacctcactatgtgctttctcagcttaccacggacaacaaaggcaactcaaaagctggaaatggaacattggacagccaaaagggaactggagtaaagaagagccctatgttgtgcggacagtatccggttaaaagtgaggggaaggagctgaagatagtggtacagcctgaaacccaacaccgagcccggtacctgacagagggcagccgaggctctgtaaaagacagaacacagcaaggctttcctacggtgaagctggaaggccataatgaaccagtggtgttgcaggtatttgtgggcaatgattctggtcgagtaaaaccacatggattctatcaggcctgtagagtaactgggcgaaatacaactccctgcaaagaagtggacattgaaggtaccactgttatagaagttggtcttgatcctagcaacaacatgacactggcggtggactgtgttggaatattgaagctgaggaatgccgatgttgaagccagaattggaattgctggatctaagaaaaagagcactcgtgccagattggtttttcgagttaatatcacaaggaaagatggctctactctgacattgcagacaccttcttcccccattttatgcactcagccagcaggagttcctgagatcttaaagaaaagcttgcatagctgttcagtgaaaggagaggaagaagtatttttaattgggaaaaactttctgaaaggaactaaagttattttccaggaaaatgtttctgatgaaaactcttggaaatcagaagctgaaattgacatggagttattccatcagaaccatcttattgtgaaggttcctccgtatcatgaccaacatataactttgcctgtatcagtgggaatatatgttgtgaccaatgctggaagatctcatgatgttcagccatttacgtacactccggatccagcagctggtgctttgaatgtaaatgtgaaaaaggagatatctagtccagcaagaccttgctcttttgaagaagccatgaaagcaatgaaaacgactggatgtaacgtagataaggtgactatccttcctaatgccctgatcactccactcatatcaagcagtatgattaagactgaagatgttactccaatggaagtaacttcagaaaaaagatcttccccaatttttcagactacaaagagcattggatcaacccagcaaaccttagaaactatttctaacatagcggggggtgcacccttctcctcaccgtcgtcatcttcccacttaactcctgaaagtgagaatcagcagcagcttcagcccaaggcatacaacccagagaccctgacaactatccagacacaggacatctcacagcccgggacctttcccgcagtttctgctgctagtcagctgcccagcagcgatgcactactgcagcaggcaacacagtttcagacaagagaagctcagtccagagacacaatacagtcagatacagtggttaacttgtcacagttaactgaagcatcacagcagcagcaatccccactacaagaacaagcacaaacgttacagcagcagataccatcaaatatttttccatcgccaagcagtgtcagccagctccagagtactattcagcagctgcaagcaggaagtttcacaggcagcgctgctggcggcaggagtggaagcgttgatttggtccagcaggttttagaggcacaacagcagttatcgtctgttctgttttctactccagatgggaatgagaatgttcaagaacagcttaatgcagacattttccaagtcagtcaaattcaaaatagtgtaagccctggaatgttttcttcagcggagtctgcagttcacactagaccagataacttactacctgggagggctgacagcgtccatcaacagactgaaaatacactgtctaatcagcagcagcagcaacagcagcagcagcaagtgatggagtcatcagctgcaatggtgatggagatgcagcagagcatttgccaagcagctgcccagatccagtcagagctgtttccttcagctgcttcagcaagtggaagccttcagcagtctccagtttaccagcagccttctcacatgatgagtgcactgcctaccaacgaggacatgcagatgcagtgtgagctgttctcgtctccccctgcagcttctggaaacgaaacttccacaaccaccacgccacaggtggcaacccctggctccaccatgtttcagacaccaagttcaggagatggagaagaaactggagcacaagcaaaacagattcagaacagcgtctttcagacaatggtccaaatgcagcgcagtggtgacagccaacctcaagtaaacctcttttcatctacaaaaaatataatgagtgttcagaataatggtacccagcaacaagggaatagcttgttccagcaagggagcgagatgatgtcacttcagtctgggaactttttgcagcagtcctctcattcacaggctcagctctttcatcctcaaaatcctattgctgatgctcagaacctttcccaggaaactcaaggttcaatttttcatagtccaaatcctattgtccacagtcaaacttccacagcatcctctgaacaactgcagccttcaatgtttcactctcagaataccattgctgtgttacagggctcttcagttcctcaagaccagcagtcacccaacatatttctttcccaaagttctatcaataatcttcaaactaacacagtagcccaagaagagcagatttcattttttgcagcacagaactcaatttctccacttcagtcaacatcaaacactgagcagcaagctgctttccaacagcagcctccaatctcacacatccagacccctattctttcccaggaacaggcacaaccctctcagcaagggttatttcagcctcaggtggcccttggctccctgcctcctaatccaatgcctcaaaaccagcaaggtccaattttccaaacacagcgcccaatagttggcatgcagagtaactctccatcccaggagcagcagcagcagcaacagcagcaacagcagcagcagcaacagcaacaacaacagcaacagagcattttattcagtaatcagaatgccatggctacaatggcctcccagaagcagccaccaccaaacatgatgtttagcccaaaccagaacccaatggctagtcaggagcagcagaaccagtcaatttttcaccagcaaagtaatatggccccaatgaaccaagagcagcagcccatgcaatttcagaatcagcctacggtttcctcacttcagaacccaggtcctacgcagtctgagtcaccacagacctccttgttccatagctctcctcagatccagttggttcaagggtcacctagttctcaagatcagcaagtaacactcttcctctctccagcatccatgtctgcattgcaaaccagtataaaccaaccagacatgcagcagtctcctctttattccccgcagaacaacatccctggaatccaaggaagcacttcttcaccccagccacaggctactttatttcacaacactacaggaggcacaataaaccaaatacaaaattctcctggctcatctcagcagacttcaggaatgttcttatttggcattcaaaataattgtagtcagcttttaacctccggaccagctacattgccagatcagctgatggccataaatcagcagggccaaccacaaaatgagggccaatcttctgtgacaacacttctttctcagcaaatgccagagacttccccactggcctcctctgtgaacagcagtcagaacatggaaaagattgatttgcttgtttcattgcaaagccaagggaacaatttaaccggctccttttagtga

SEQ ID NO: 11 - DNA, 22 bp, Artificial
  884 bp NFAT 5′ probe
ttcgctaccatactggaaaagg

SEQ ID NO: 12 - DNA, 23 bp, Artificial
  884 bp 5′ probe
gatttgtgaactgattgctttcc

SEQ ID NO: 13 - DNA, 22 bp, Artificial
  672 bp NFAT 3′ probe
acactcaagaatcagaggcagg

SEQ ID NO: 14 - DNA, 22 bp, Artificial
  672 bp NFAT 3′ probe
tcttgtttctgctcctagtccc

SEQ ID NO: 15 - DNA, 20 bp, Artificial
  NFAT knockout allele primer
aacttgccctgtcagtcacc

SEQ ID NO: 16 - DNA, 27 bp, Artificial
  NFAT knockout allele primer
gggctatagacatgcaccaccacacag

SEQ ID NO: 17 - DNA, 20 bp, Artificial
  NFAT wildtype allele primer
aagggcttcttccagaatgg

(19) United States
(12) Patent Application Publication
(10) Pub. No.: US 2008/0039416 A1
(43) Pub. Date: Feb. 14, 2008

(54) Methods for Treating Disease by Modulating an Osmotic Stress Pathway

(76) Inventor: Steffan N. Ho, San Diego, CA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/569,756

(22) PCT Filed: Jun. 2, 2005
(86) PCT No.: PCT/US05/19432

§ 371 Date: Apr. 25, 2007

Related U.S. Application Data
(60) Provisional application No. 60/576,419, filed on Jun. 2, 2004.

(51) Int. Cl. A61K 31/7052; A01K 67/027; A61P 35/00; A61P 37/00; C07H 21/04; C12Q 1/18; C12Q 1/68; G01N 33/566; G01N 33/574

(52) U.S. Cl. 514/44; 435/32; 435/6; 435/7.23; 436/501; 514/789; 536/23.1; 536/23.5; 800/13

(57) ABSTRACT

The cellular response to osmotic stress ensures that the concentration of water inside the cell is maintained within a range that is compatible with biologic function. Single cell organisms are particularly dependent on mechanisms that permit adaptation to osmotic stress because each individual cell is directly exposed to the external environment. Mammals, however, limit osmotic stress by establishing an internal aqueous environment in which intravascular water and electrolytes are subject to sensitive and dynamic, organism-based homeostatic regulation. NFAT5/TonEBP is an essential mammalian osmoregulatory transcription factor, and this invention demonstrates the unexpected yet critical significance of cell-based osmotic regulation in vivo. The invention highlights the fundamental importance of maintaining intracellular water homeostasis in the face of varying cellular metabolic activity and distinct tissue microenvironments. Methods for treating, preventing, or inhibiting human diseases using the osmotic stress pathway have been provided.

### CROSS-REFERENCE TO RELATED APPLICATIONS

This application claims priority from U.S. Provisional Application Ser. No. 60/576,419 filed on Jun. 2, 2004, which is incorporated herein by reference in its entirety.

### STATEMENT REGARDING FEDERALLY SPONSORED RESEARCH OR DEVELOPMENT

This invention was made in part with Government support under Natonal Institutes of Health Grant GM59651. The Government has certain rights in the invention

### INCORPORATION-BY-REFERENCE OF MATERIAL SUBMITTED ON A COMPACT DISC

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### BACKGROUND OF THE INVENTION

1. Field of the Invention

The current invention generally relates to osmotic stress pathways, the molecules that play key roles in those pathways, as well as methods for screening, diagnosing, inhibiting, and treating human diseases by targeting the molecular compounds of the osmotic stress pathway.

2. Description of Related Art

Cells regulate osmolarity by controlling the solute concentrations inside the cell. Changes in osmolarity can lead to potentially detrimental changes in cell volume and ionic strength. In the living cell, changes in osmolyte concentration occurring either within or outside the cell can lead to a rapid flow of water across the plasma membrane, resulting in marked alterations in cell volume. Exposure of a cell to a hypertonic environment in which the osmolality outside the cell is greater than that present within the cell results in the osmotic efflux of water, a reduction in cell volume (i.e., cell shrinkage) and an increase in the concentration of all intracellular constituents. The resulting biochemical disequilibrium gives rise to a wide spectrum of deleterious effects on cell function and, in the event biochemical homeostasis is not restored, ultimately results in apoptotic cell death.

Hyperosmotic stress in mammalian systems is known in the field as being important to the physiology of the kidney—the mechanisms involved in concentrating urine through the regulated retention or excretion of water and electrolytes within the medulla of the kidney take advantage of osmotic stress in their normal functioning. However, the osmolarity of normal tissues is assumed to be identical to that of blood, and therefore, the study of the osmotic stress pathway has not been well elucidated. NFAT5/TonEBP, a transcription factor that contains the rel DNA binding domain also found in rel/NFκB/NFAT family of transcription proteins which controls the transcription of genes, is the only known osmo-sensitive mammalian transcription factor that is activated in response to hypertonicity. NFAT5/TonEBP is known to comprise three human isoforms of which “isoform a” has been characterized by X-Ray crystallography.

Cancers and autoimmune diseases are well-known killers. Much research has been conducted to obtain a cure, or at least partially arrest cancers and autoimmune diseases, but success has been limited. What is needed, therefore, is a novel approach to studying and providing treatments and therapeutics for the management of cancers and autoimmune diseases.

### BRIEF SUMMARY OF THE INVENTION

The invention provides a method for identifying a candidate anti-cancer or immunosuppressive compound, the method comprising (a) culturing a first cell and a second cell under conditions of hypertonic stress; (b) contacting the first cell with a test agent; and (c) assaying the first cell and the second cell for cell growth or viability, wherein a decrease in cell growth or viability in the first cell relative to the second cell indicates that the test agent is a candidate anti-cancer or immunosuppressive compound. In various aspects, the first and second cell can comprise an NFAT5/TonEBP polynucleotide or polypeptide. In various other aspects, the first and second cell can comprise a NFAT5/TonEBP variant polynucleotide. In yet another aspect, the first and second cell can comprise a NFAT5/TonEBP variant or fragment protein. The assay for cell growth or viability can comprise an assay selected from the group consisting of a cell uptake assay, cell binding assay, growth inhibition assay and any combination thereof.

In another aspect of the present invention, a method is provided for identifying a candidate anti-cancer or immunosuppressive compound, the method comprising: (a) contacting NFAT5/TonEBP or biologicallyactive fragment with a known compound that binds NFAT5/TonEBP to form an assay mixture, and (b) contacting the assay mixture with a test compound, and determining the ability of the test compound to interact with NFAT5/TonEBP.

In another aspect of the present invention, a method is provided for identifying a candidate anti-cancer or immunosuppressive compound, the method comprising (a) performing a structure based drug design using a three dimensional structure determined for a crystal of NFAT5/TonEBP. The three dimensional structure can comprise atomic coordinates of Protein Data Bank Accession No. 1IMH. The method may further comprise (b) contacting the candidate inhibitor with a cell comprising NFAT5/TonEBP or a variant or fragment thereof; and (c) detecting inhibition of at least one activity of NFAT5/TonEBP, variant or fragment thereof. Alternatively, the method may further comprise (b) contacting the candidate inhibitor with a cell comprising NFAT5/TonEBP or a variant or fragment thereof; (c) culturing the cell under conditions of hypertonic stress; (d) contacting the cell with the candidate compound; and (e) assaying the cell for cell growth or viability. In various aspects, the assay for cell growth or viability can comprise an assay selected from the group consisting of a cell uptake assay, cell binding assay, growth inhibition assay and any combination thereof.

In yet another aspect of the present invention, a method is provided for treating a cancer or an autoimmune disease in a subject, the method comprising administering to a subject in need thereof a therapeutically effective amount of an inhibitor of NFAT5/TonEBP. In various aspects, the inhibitor is selected by (a) culturing a first cell and a second cell under conditions of hypertonic stress; (b) contacting the first cell with a test agent; and (c) assaying the first cell and the second cell for cell growth or viability, wherein a decrease in cell growth or viability in the first cell relative to the second cell indicates that the test agent is an inhibitor of NFAT5/TonEBP. In various aspects, the first and second cell can comprise an NFAT5/TonEBP polynucleotide or polypeptide. In various other aspects, the first and second cell can comprise a NFAT5/TonEBP variant polynucleotide. In yet another aspect, the first and second cell can comprise a NFAT5/TonEBP variant or fragment protein. The assay for cell growth or viability can comprise an assay selected from the group consisting of a cell uptake assay, cell binding assay, growth inhibition assay and any combination thereof.

In another aspect of the present invention, the inhibitor is selected by: (a) performing a structure based drug design using a three dimensional structure determined for a crystal of NFAT5/TonEBP. The three dimensional structure can comprise atomic coordinates of Protein Data Bank Accession No. 1IMH. The method may further comprise (b) contacting the candidate inhibitor with a cell comprising NFAT5/TonEBP or a variant or fragment thereof; and (c) detecting inhibition of at least one activity of NFAT5/TonEBP, variant or fragment thereof. Alternatively, the method may further comprise (b) contacting the candidate inhibitor with a cell comprising NFAT5/TonEBP or a variant or fragment thereof; (c) culturing the cell under conditions of hypertonic stress; (d) contacting the cell with the candidate compound; and (e) assaying the cell for cell growth or viability. In various aspects, the assay for cell growth or viability can comprise an assay selected from the group consisting of a cell uptake assay, cell binding assay, growth inhibition assay and any combination thereof. In various aspects, the cell can comprise a NFAT5/TonEBP variant polynucleotide or NFAT5/TonEBP variant or fragment protein. In various aspects, the inhibitor is an antibody directed against NFAT5/TonEBP. In various aspects, the antibody can be a monoclonal antibody. In various aspects, the inhibitor can be selected from the group consisting of a ribozyme, antisense compound, triplex-forming molecule, siRNA, and aptamer. In various aspects, the cancer can be selected from the group consisting of epithelial malignancies, sarcomas, and lymphomas. In various aspects, the autoimmune disease can be selected from the group consisting of rheumatoid arthritis, systemic lupus erythematosis, multiple sclerosis, and type I diabetes.

In yet another aspect of the present invention, a method is provided for diagnosing a cancer or an autoimmune disease in a subject comprising (a) obtaining a sample from the subject; (b) detecting NFAT5/TonEBP expression in the sample; and (c) comparing to the expression of NFAT5/TonEBP of the sample to a control sample, wherein an elevated expression of NFAT5/TonEBP in the sample is diagnostic for cancer. In various aspects, (b) can comprise detecting NFAT5/TonEBP mRNA. Alternatively, (b) can comprise detection of NFAT5/TonEBP protein.

In another aspect of the present invention, a method for treating a cancer or an autoimmune disease is provided comprising decreasing the activity of NFAT5/TonEBP. In various aspects, decreasing the activity can comprise decreasing the expression of NFAT5/TonEBP. In various aspects, decreasing the expression comprises transforming a cell to express a polynucleotide anti-sense to at least a portion of an endogenous polynucleotide encoding NFAT5/TonEBP. In various aspects, decreasing the activity comprises inhibiting, preventing, or reversing at least one activity of NFAT5/TonEBP. In various other aspects, decreasing the activity can comprise transforming a cell to express an aptamer to NFAT5/TonEBP. In various aspects, decreasing the activity can comprise introducing into a cell an aptamer to NFAT5/TonEBP. In a further aspect, decreasing the activity can comprise administering to a cell an antibody that selectively binds NFAT5/TonEBP. In various aspects, the cancer can be selected from the group consisting of epithelial malignancies, sarcomas, and lymphomas. In various aspects, the autoimmune disease can be selected from the group consisting of rheumatoid arthritis, systemic lupus erythematosis, multiple sclerosis, and type I diabetes.

In yet another aspect of the present invention, a method is provided for determining whether a compound up-regulates or down-regulates the transcription of a NFAT5/TonEBP gene, comprising contacting the compound with a RNA polymerase and said gene, followed by measuring NFAT5/TonEBP gene transcription initiated by the RNA polymerase acting on the gene, wherein measuring enhanced transcription is indicative of up-regulation and measuring decreased transcription is indicative of down-regulation. In various aspects, the contacting can occur in a cell. In another aspect, the compound can be selected from the group consisting of a ribozyme, antisense compound, triplex-forming molecule, siRNA, and aptamer.

In another aspect of the present invention, a method is provided for determining whether a compound up-regulates or down-regulates translation of an NFAT5/TonEBP gene in a cell, comprising contacting the compound with the cell, the cell further comprising the NFAT5/TonEBP gene, and measuring NFAT5/TonEBP gene translation, wherein measuring enhanced translation is indicative of up-regulation and measuring decreased translation is indicative of down-regulation. In various aspects, the compound can be selected from the group consisting of a ribozyme, antisense compound, triplex-forming molecule, siRNA, and aptamer.

In a further aspect of the present invention, a method is provided for determining whether a compound is a NFAT5/TonEBP target gene inhibitor, comprising: (a) culturing a first cell and a second cell under conditions of hypertonic stress; (b) contacting the first cell with a test agent; and (c) assaying the first cell and the second cell for at least one activity of a NFAT5/TonEBP target gene, wherein a decrease in activity of a NFAT5/TonEBP target gene in the first cell relative to the second cell indicates that the test agent is an NFAT5/TonEBP target gene inhibitor. In various aspects, the contacting can occur in a cell. In another aspect, the compound can be selected from the group consisting of a ribozyme, antisense compound, triplex-forming molecule, siRNA, and aptamer. In various aspects, the NFAT5/TonEBP target gene can be selected from the group consisting of aldose reductase, sodium/myo-inositol cotransporter, sodium/chloride/betaine cotransporter, urea transporter, taurine transporter, heat shock protein 70 gene, sodium-coupled neutral amino acid transporter-2, osmotic stress protein of 94 kDa and aquaporin 2.

In yet another aspect of the present invention, a non-human transgenic animal is provided, wherein at bast one NFAT5/TonEBP gene comprised by the non-human transgenic animal is disrupted. In various aspects, the NFAT5/TonEBP gene is not fully expressed. In another aspect, the non-human animal can be selected from the group consisting of a mouse, rat, dog, cat, cow, pig, horse, rabbit, frog, chicken, and sheep. In one aspect, the non-human transgenic animal is a mouse.

In a further aspect of the present invention, a polynucleotide is provided comprising a nucleotide sequence selected from the group consisting of SEQ ID NO: 2, SEQ ID NO: 4, SEQ ID NO: 6, SEQ ID NO: 8, and SEQ ID NO: 10, and wherein the polynucleotide lacks exons 6 and 7. In another aspect of the present invention, a polynucleotide is provided comprising at least about 80% sequence identity to a polynucleotide comprising a nucleotide sequence selected from the group consisting of SEQ ID NO: 2, SEQ ID NO: 4, SEQ ID NO: 6, SEQ ID NO: 8, and SEQ ID NO: 10, and wherein the polynucleotide encodes a polypeptide comprising at least one activity of an NFAT5/TonEBP protein.

In another aspect of the present invention, a polypeptide is provided which is expressed from the polynucleotide provided above. In another aspect of the present invention, a polypeptide is provided comprising an amino acid sequence selected from the group consisting of SEQ ID NO: 1, SEQ ID NO: 3, SEQ ID NO: 5, SEQ ID NO: 7, and SEQ ID NO: 9, and wherein the polypeptide lacks an amino acid sequence encoded by exons 6 and 7 of an NFAT5/TonEBP gene. In yet another aspect of the present invention, a polypeptide is provided having at least about 50% sequence identity to a polypeptide comprising an amino acid sequence selected from the group consisting of SEQ ID NO: 1, SEQ ID NO: 3, SEQ ID NO: 5, SEQ ID NO: 7, and SEQ ID NO: 9, and wherein the polypeptide comprises at least one activity of an NFAT5/TonEBP protein.

In a further aspect of the present invention, a vector is provided comprising the polynucleotide provided above. In another aspect, a cell is provided comprising any of the polynucleotides provided above. In yet another aspect, a tissue is provided comprising the cell provided above. In another aspect, an organism is provided comprising the cell provided above. In a further aspect, an organism is provided comprising a cell capable of expressing the polypeptides of any of the polypeptides provided above.

In a further aspect of the present invention, an anti-cancer or immunosuppressive compound is provided which is identified by the method comprising: (a) culturing a first cell and a second cell under conditions of hypertonic stress; (b) contacting the first cell with a test agent; and (c) assaying the first cell and the second cell for cell growth or viability, wherein a decrease in cell growth or viability in the first cell relative to the second cell indicates that the test agent is an anti-cancer or immunosuppressive compound. In various aspects, the first and second cell can comprise an NFAT5/TonEBP polynucleotide or polypeptide. In various aspects, the first and second cell comprise a NFAT5/TonEBP variant polynucleotide. In various aspects, the first and second cell comprise a NFAT5/TonEBP variant or fragment protein. In other aspects, the assay for cell growth or viability can comprise an assay selected from the group consisting of a cell uptake assay, cell binding assay, growth inhibition assay and any combination thereof.

In yet another aspect of the present invention, an anti-cancer or immunosuppressive compound is provided which is identified by the method comprising: (a) performing a structure based drug design using a three dimensional structure determined for a crystal of NFAT5/TonEBP. In various aspects, the three dimensional structure can comprise atomic coordinates of Protein Data Bank Accession No. 1IMH. In some aspects, the method can further comprise: (b) contacting the candidate inhibitor with a cell comprising NFAT5/TonEBP or a variant or fragment thereof; and (c) detecting inhibition of at least one activity of NFAT5/TonEBP, variant or fragment thereof. Alternatively, the method can further comprise: (b) contacting the candidate inhibitor with a cell comprising NFAT5/TonEBP or a variant or fragment thereof; (c) culturing the cell under conditions of hypertonic stress; (d) contacting the cell with the candidate compound; and (e) assaying the cell for cell growth or viability. In various aspects, the assay for cell growth or viability can comprise an assay selected from the group consisting of a cell uptake assay, cell binding assay, growth inhibition assay and any combination thereof. The compounds identified above can be a pro-drug or pharmaceutically acceptable salt of the compound, or combination with a pharmaceutically acceptable carrier.

These and other features, aspects and advantages of the present invention will become better understood with reference to the following description, examples and appended claims.

### BRIEF DESCRIPTION OF THE SEVERAL VIEWS OF THE DRAWINGS

FIG. 1. Depicted is a schematic representation of a gene-targeting strategy for deletion of exons 6 and 7 of the Nfat5 gene by homologous recombination in embryonic stem cells. The top line represents the wild type Nfat5 gene with exons and points of recombination indicated: A is AvrII, R is EcoRI, X is XhoI, and M is MscI. The middle line represents a gene-targeting vector containing three IoxP recombination sites and a neomycin-selectable marker within a 12.4-kb genomic region encompassing exons 5-7. The bottom line represents the resultant deletion mutant, Nfat5^{Δ}.

FIG. 2. Depicted is a schematic representation of wild type Nfat5, indicating the points of the nucleotide sequence that map on to the DNA binding domain of the expressed protein (upper panel). The lower panel is a structural depiction of the region of the NFAT5/TonEBP DNA-binding domain targeted for deletion. The targeted deletion eliminates exons 6 and 7, which encode amino acids comprising the N-terminal DNA-binding loop (1). Deleted amino acids are shown in green, the remainder of the DNA-binding domain is blue, and DNA is colored red and yellow.

FIG. 3. Depicted are the results of PCR genotyping verification of Nfat5 and the deletion mutant mRNA encoded by exons 5-8 (top panel) in MEF cell lines, and Southern blot analysis of Nfat5 and the deletion mutant (lower panel), confirming germ line transmission of the allele bearing the exon 6 and 7 deletion.

FIG. 4. Depicted is RT-PCR analysis of RNA isolated from SV40 T antigen-immortalized embryonic fibroblast (MEF) cell lines derived from Nfat5^{+/+}, Nfat5^{+/Δ} and Nfat5^{Δ/Δ} embryos (right panel). The left panel depicts schematic representations of both the wild type and deletion mutants. The arrows above each graphic indicate the RT-PCR analysis primers positioned as indicated relative to that portion of the NFAT5/TonEBP mRNA encoded by exons 5-8.

FIG. 5. Whole cell extracts from the Nfat5^{+/+}, Nfat5^{+/Δ} and Nfat5^{Δ/Δ} MEF cell lines cultured under either standard or hypertonic (HI) culture conditions. The Western immunoblot analysis was conducted using the indicated primary antisera. (ns) refers to the control nonspecific bands.

FIG. 6. Depicted are graphs representing luciferase reporter activity (relative light units, RLU) in immortalized MEF cell lines derived from wild type, heterozygous and homozygous deletion mutant embryos. The pictured data are representative of three independent experiments. (A) depicts data from experiments using the NFAT5/TonEBP reporter and (B) depicts data from experiments using the hsp70.1 reporter. Both sets of data were generated from cells under hypertonic (added NaCl) and isotonic (added raffinose) as indicated.

FIG. 7. Depicted are cell counts from the indicated tissue for wild type and heterozygous mice.

FIG. 8. Depicted is a Western Blot analysis of cells from Nfat5^{+/Δ} and Nfat5+/+ mice. (ns) refers to the control nonspecific bands.

FIG. 9. Depicted are ELISA data showing relative antigen-specific immunoglobulin levels of Nfat5^{+/Δ} and Nfat5^{+/+} mice immunized with ovalbumin.

FIG. 10. Depicted are data quantifying the proliferative responses of Nfat5+/Δ lymphocytes in isotonic and hypertonic conditions. Anti-CD3/CD28 refers to stimulation of T-cells, while LPS refers to stimulation of B cells. FIG. 11. Shown is the growth immortalized embryonic fibroblast (MEF) cell lines derived from Nfat5^{+/+}, Nfat5^{+/Δ} and Nfat5^{Δ/Δ} embryos under the indicated osmotic conditions.

FIG. 12. Depicted are cell counts for MEF cell lines derived from the indicated embryos at two different osmostic conditions.

FIG. 13. Depicted is the osmolality of indicated mouse lymphoid tissues.

FIG. 14. Depicted are cell doublings number for wild type (wt—filled squares), heterozygous (HT—filled diamond) and knockout (KO—open squares) MEF cell lines derived from mouse embryos.

FIG. 15. Depicted is SDS-PAGE analysis of normal and malignant prostate tissue.

FIG. 16. Depicted is SDS-PAGE analysis of protein extracts of tumor nodules from nude mice engrafted with U87 human glioma cancer cells.

FIG. 17. Depicted is Western blot analysis of tissues extracted from mice. A murine thymocyte cell suspension included as a control (lane 1, thy) can be compared to test samples: lane 3, infiltrating ductal carcinoma; lane 5, adenocarcinoma (AdCa), likely metastatic; lane 6, adenocarcinoma, unknown primary; lane 7, squamous cell carcinoma (SCC); lane 8, metastatic SCC from the lung; lane 9, metastatic poorly differentiated adenocarcinoma from the breast; lanes 11, 13, 15, 17, metastatic colorectal adenocarcinoma. NL refers to normal adjacent tissue; ca refers to cancer tissue. F=female; M=male.

FIG. 18. Depicted are data representing mean tumor size in a xenograft assay. Cell lines represent the indicated genotypes, and cell lines designated 2.1 were generated independently (on a separate day) of those designated 2.2.

### DETAILED DESCRIPTION OF THE INVENTION

Abbreviations and Definitions

To facilitate understanding of the invention, a number of terms and abbreviations as used herein are defined below as follows:

To distinguish between genes (and related nucleic acids) and the proteins that they encode, the abbreviations for genes are indicated by italicized text while abbreviations for the proteins start with a capital letter and are not italicized. Thus, NFAT5/TonEBP refers to the nucleotide sequence that encodes NFAT5/TonEBP.

Abs: As used herein, the term “Abs” refers to antibodies which may be single anti-NFAT5/TonEBP monoclonal Abs (including agonist, antagonist and neutralizing Abs), anti-NFAT5/TonEBP antibody compositions with polyepitopic specificity, single chain anti-NFAT5/TonEBP Abs, and fragments of anti-NFAT5/TonEBP Abs. A “monoclonal antibody” refers to an antibody obtained from a population of substantially homogeneous Abs, i.e., the individual Abs comprising the population are identical except for naturally-occurring mutations that may be present in minor amounts

Active Polypeptide: As used herein, the term “active polypeptide” refers to a NFAT5/TonEBP, NFAT5/TonEBP fragment or NFAT5/TonEBP variant which retains a biological and/or an immunological activity of native or naturally occurring NFAT5/TonEBP. Immunological activity refers to the ability to induce the production of an antibody against an antigenic epitope possessed by a native NFAT5/TonEBP; biological activity refers to a function, either inhibitory or stimulatory, caused by a native NFAT5/TonEBP that excludes immunological activity. A biological activity of NFAT5/TonEBP includes, for example, its regulation of target genes disclosed herein.

Control Sequences: As used herein, the term “control sequences” refers to DNA sequences that enable the expression of an operably-linked coding sequence in a particular host organism. Prokaryotic control sequences include promoters, operator sequences, and ribosome binding sites. Eukaryotic cells utilize promoters, polyadenylation signals, and enhancers.

Controlled-Release Component: As used herein, the term “controlled-release component” refers to a composition or compound, including, but not limited to, polymers, polymer matrices, gels, permeable membranes, liposomes, microspheres, or the like, or a combination thereof, that facilitates the controlled-release of a composition or composition combination.

Epitope Tag: As used herein, the term “epitope tag” refers to a chimeric polypeptide fused to a “tag polypeptide”. Such tags provide epitopes against which Abs can be made or are available, but do not interfere with polypeptide activity. To reduce anti-tag antibody reactivity with endogenous epitopes, the tag polypeptide is preferably unique. Suitable tag polypeptides generally have at least six amino acid residues and usually between about 8 and 50 amino acid residues, preferably between 8 and 20 amino acid residues). Examples of epitope tag sequences include HA from Influenza A virus and FLAG.

Isolated: As used herein, the term “isolated,” when referred to a molecule, refers to a molecule that has been identified and separated and/or recovered from a component of its natural environment. Contaminant components of its natural environment are materials that interfere with diagnostic or therapeutic use.

Isolated Nucleic Acid: As used herein, the term “isolated nucleic acid” refers to a molecule purified from the setting in which it is found in nature and is separated from at least one contaminant nucleic acid molecule. Isolated NFAT5/TonEBP molecules are distinguished from the specific NFAT5/TonEBP molecule, as it exists in cells. However, an isolated NFAT5/TonEBP molecule includes NFAT5/TonEBP molecules contained in cells that ordinarily express the NFAT5/TonEBP where, for example, the nucleic acid molecule is in a chromosomal location different from that of natural cells.

Isolated or Purified Polypeptide, Protein or Fragment: As used herein, the terms “isolated” or “purified” polypeptide, protein or biologically active fragment refer to those items separated and/or recovered from a component of its natural environment. Contaminant components include materials that would typically interfere with diagnostic or therapeutic uses for the polypeptide, and may include enzymes, hormones, and other proteinaceous or non-proteinaceous materials. Preferably, the polypeptide is purified to a sufficient degree to obtain at least 15 residues of N-terminal or internal amino acid sequence. To be substantially isolated, preparations having less than 30% by dry weight of non-NFAT5/TonEBP contaminating material (contaminants), more preferably less than 20%, 10% and most preferably less than 5% contaminants. An isolated, recombinantly-produced NFAT5/TonEBP or biologically active portion is preferably substantially free of culture medium, i.e., culture medium represents less than 20%, more preferably less than about 10%, and most preferably less than about 5% of the volume of the NFAT5/TonEBP preparation. Examples of contaminants include cell debris, culture media, and substances used and produced during in vitro synthesis of NFAT5/TonEBP.

Operably Linked: As used herein, the term “operably linked” refers to nucleic acid when it is placed into a functional relationship with another nucleic acid sequence. For example, a promoter or enhancer is operably linked to a coding sequence if it affects the transcription of the sequence, or a ribosome-binding site is operably-linked to a coding sequence if positioned to facilitate translation. Generally, “operably-linked” means that the DNA sequences being linked are contiguous, and, in the case of a secretory leader, contiguous and in reading phase. However, enhancers do not have to be contiguous. Linking is accomplished by conventional recombinant DNA methods.

Pharmaceutically Acceptable: As used herein, the term “pharmaceutically acceptable” means approved by a regulatory agency of the Federal or a state government or listed in the U.S. Pharmacopeia or other generally recognized pharmacopeia for use in animals, and more particularly in humans.

Pharmaceutically Acceptable Carrier: As used herein, the term “pharmaceutically acceptable carrier” refers to a diluent, adjuvant, excipient, or vehicle with which a composition is administered. Such carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when a composition is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier.

Pharmaceutically Acceptable Salt: As used herein, the term “pharmaceutically acceptable salt” includes those salts of a pharmaceutically acceptable composition formed with free amino groups such as those derived from hydrochloric, phosphoric, acetic, oxalic, tartaric acids, and those formed with free carboxyl groups such as those derived from sodium, potassium, ammonium, calcium, ferric hydroxides, isopropylamine, triethylamine, 2-ethylamino ethanol, histidine, and procaine. If the composition is basic, salts may be prepared from pharmaceutically acceptable non-toxic acids including inorganic and organic acids. Such acids include acetic, benzene-sulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. Particularly preferred are besylate, hydrobromic, hydrochloric, phosphoric and sulfuric acids. If the composition is acidic, salts may be prepared from pharmaceutically acceptable organic and inorganic bases. Suitable organic bases include, but are not limited to, lysine, N,N′-dibenzylethylenediamine, chloroprocaine, choline, diethanolamine, ethylenediamine, meglumine (N-methylglucamine) and procaine. Suitable inorganic bases include, but are not limited to, alkaline and earth-alkaline metals such as aluminum, calcium, lithium, magnesium, potassium, sodium and zinc.

Pro-Drug: As used herein, the term “pro-drug” refers to any composition which releases an active drug in vivo when such a composition is administered to a mammalian subject. Pro-drugs can be prepared, for example, by functional group modification of a parent drug. The functional group may be cleaved in vivo to release the active parent drug compound. Pro-drugs include, for example, compounds in which a group that may be cleaved in vivo is attached to a hydroxy, amino or carboxyl group in the active drug. Examples of pro-drugs include, but are not limited to esters (e.g., acetate, methyl, ethyl, formate, and benzoate derivatives), carbamates, amides and ethers. Methods for synthesizing such pro-drugs are known to those of skill in the art.

Purified Polypeptide: As used herein, the term “purified polypeptide” refers to a molecule which is purified (1) to obtain at least 15 residues of N-terminal or internal amino acid sequence using a sequenator, or (2) to homogeneity by SDS-PAGE under non-reducing or reducing conditions using Coomassie blue or silver stain. Isolated polypeptides include those expressed heterologously in genetically-engineered cells or expressed in vitro, since at least one component of the NFAT5/TonEBP natural environment will not be present. Ordinarily, isolated polypeptides are prepared by at least one purification step.

Therapeutically Effective Amount: As used herein, the term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition. More specific embodiments are included in the Pharmaceutical Preparations and Methods of Administration section below.

Methods for Treating Disease by Modulating an Osmotic Stress Pathway

The present invention utilizes the discovery that osmotic stress response is a critical factor in the regulation of cancers and autoimmune diseases. The invention herein provides a mode of treating cancers and autoimmune diseases in human and other subjects by identifying and administering an osmotic stress response, in particular a NFAT5/TonEBP, inhibitor. The invention also provides polynucleotides, polypeptides, vectors, cells, tissues and organisms useful in the identification and treatment of cancers and autoimmune diseases. A number of desirable anti-cancer and immunosuppressive aspects are achieved by various embodiments of the present invention.

The osmotic stress response pathway has been discovered to be necessary for cellular proliferation and immune cell. Immune cells and are also dependent on this pathway—a mutation in the mouse genome results in either complete or partial loss of the function of NFAT5/TonEBP as a DNA binding transcription factor. While complete loss of function resulted in late gestational/neonatal lethality and severe impairment of cell proliferation under hyperosmotic culture conditions, animals exhibiting partial loss of function were viable and demonstrated defects in adaptive immunity.

Direct measurements of NFAT5/TonEBP and osmotic stress reveal lymphoid tissues to be hyperosmolar relative to serum. This discovery not only demonstrates that NFAT5/TonEBP represents a critical component of the mammalian osmotic stress response, but also provides new insight into the lymphoid tissue microenvironment, thus highlighting the broader biologic significance of osmotic stress and the NFAT5/TonEBP osmotic stress response pathway in vivo.

The unique microenvironment within a tumor, characterized by a complete absence of functional intra-tumor lymphatics, renders the growth and survival of malignant cells in vivo critically dependent on the mammalian osmotic stress response pathway. The osmotic stress response pathway represents a highly specific target for anticancer drug discovery. All normal tissues contain lymphatic vessels that allow for the removal of membrane impermeable solutes (i.e., osmolytes) that accumulate within the interstitial space. However, there are no functional lymphatics within tumors. Inhibition of the cellular osmotic stress response pathway in tumor tissue results in cell death due to hypertonic stress. In the absence of functional lymphatics, cell death exacerbates osmotic stress, which enhances cell death, thus establishing an osmolytic feedback loop.

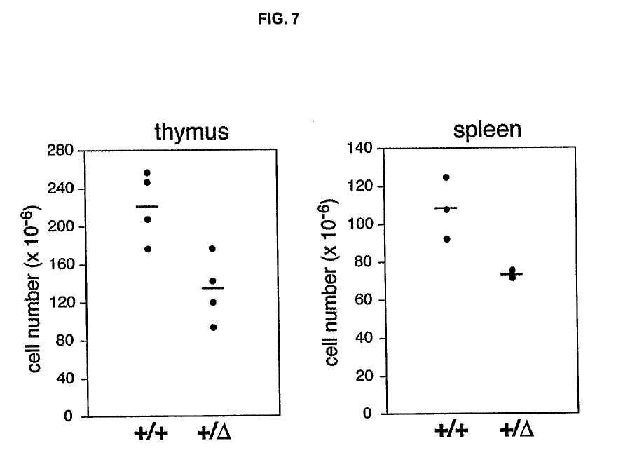

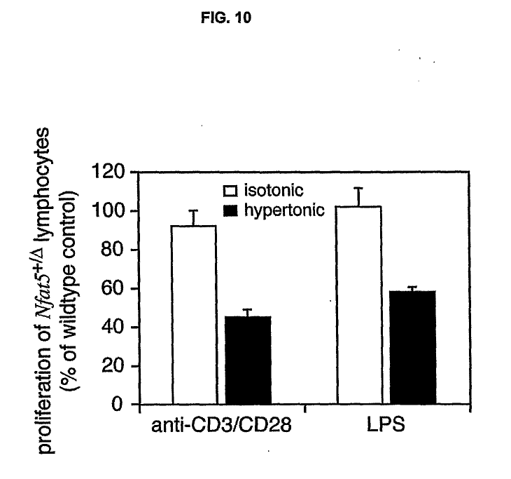

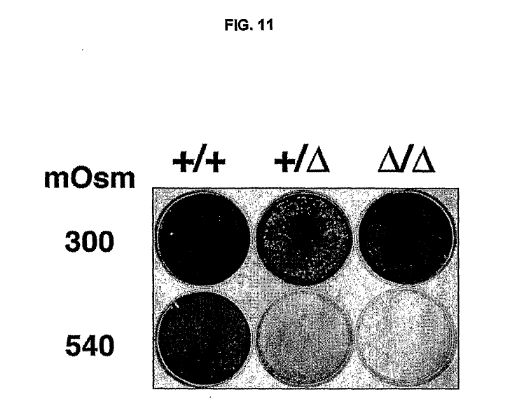

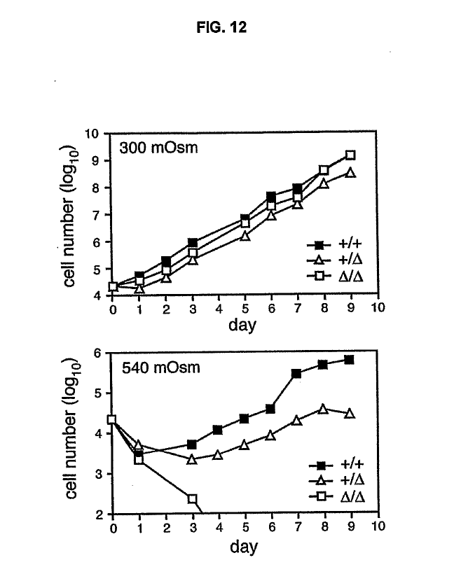

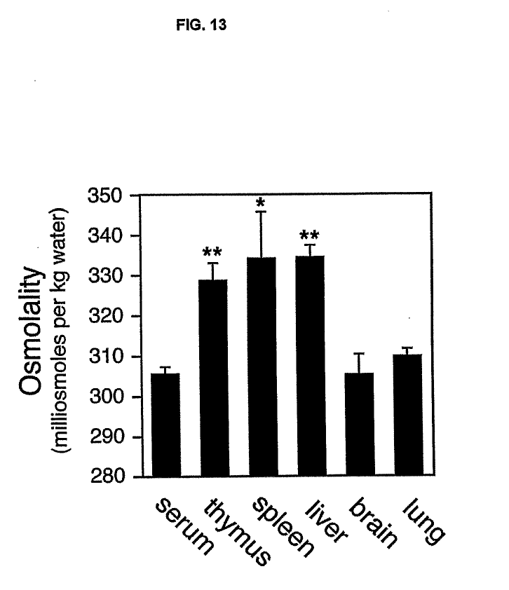

In addition, lymphoid tissue hypocellularity and impaired adaptive immune response observed in Nfat5^{+/Δ} mice (FIG. 7) indicate that NFAT5/TonEBP also plays a critical role in the function of the adaptive immune system. The observed correlation between the impaired lymphocyte responses in vivo resulting from partial loss of NFAT5/TonEBP function and the impaired proliferative responses of Nfat5^{+/Δ} lymphocytes ex vivo observed upon culture under conditions of hyperosmotic stress (FIG. 10) provides that NFAT5/TonEBP functions as part of an osmotic stress response pathway in lymphoid tissues. This conclusion is supported by the observation that lymphoid tissues are hyperosmotic relative to blood based on direct measurements of tissue osmolality (FIG. 13).

The late embryonic/neonatal lethality observed in both homozygous null and homozygous Nfat5^{Δ/Δ} animals definitively demonstrates a critical role for NFAT5/TonEBP outside the kidney. Therefore, those of skill in the art will recognize that NFAT5/TonEBP is a target for drug discovery and designing new treatments for cancers and autoimmune diseases.

Accordingly, the invention provides a method for identifying a candidate anti-cancer or immunosuppressive compound, the method comprising (a) culturing a first cell and a second cell under conditions of hypertonic stress; (b) contacting the first cell with a test agent; and (c) assaying the first cell and the second cell for cell growth or viability. The decrease in cell growth or viability in the first cell relative to the second cell indicates that the test agent is a candidate anticancer or immunosuppressive compound. In various aspects, the first and second cell of the compound identification method comprise an NFAT5/TonEBP polynucleotide or polypeptide as a mode for obtaining information on test agents which may be identified as osmotic stress inhibitors, and thus anti-cancer or immunosuppressive compounds.

The NFAT5/TonEBP polypeptide and polynucleotide sequences for human (SEQ ID NOs: 1-6) and murine (SEQ ID NOs: 7-10) are provided. SEQ ID NOs: 1, 3 and 5 provide human isoforms a, b and c of NFAT5/TonEBP. SEQ ID NOs: 2, 4 and 6 provide polynucleotide sequences, or genes, encoding human isoforms a, b and c. Likewise, SEQ ID NOs: 7 and 9 provide murine isoforms a and b of NFAT5/TonEBP. SEQ ID NOs: 8 and 10 provide polynucleotide sequences encoding murine isoforms a and b.

In one embodiment, an isolated NFAT5/TonEBP molecule can be used to express NFAT5/TonEBP (e.g., via a recombinant expression vector in a host cell in gene therapy applications), to detect NFAT5/TonEBP mRNA (e.g., in a biological sample) or to modulate an NFAT5/TonEBP activity. In addition, NFAT5/TonEBP polypeptides, e.g., SEQ ID NOs: 1, 3, 5, 7 and 9, can be used to screen drugs or compounds that modulate the NFAT5/TonEBP activity or expression as well as to treat disorders characterized by insufficient or excessive production (such as cancer) of NFAT5/TonEBP or production of NFAT5/TonEBP forms that have decreased or aberrant activity compared to NFAT5/TonEBP wild-type protein, or modulate biological function that involve NFAT5/TonEBP. In addition, the anti-NFAT5/TonEBP Abs of the invention can be used to detect and isolate NFAT5/TonEBP and modulate NFAT5/TonEBP activity.

Accordingly, the present invention provides a method for identifying a candidate anti-cancer or immunosuppressive compound, i.e., candidate or test compounds or agents (e.g., peptides, peptidomimetics, small molecules or other drugs), and combinations thereof, that effect NFAT5/TonEBP, a stimulatory or inhibitory effect, including translation, transcription, activity or copies of the gene in cells. The present invention also includes compounds identified in screening assays.

Testing for compounds that increase or decrease NFAT5/TonEBP activity are desirable. A compound may modulate NFAT5/TonEBP activity by affecting: (1) the number of copies of the gene in the cell (amplifiers and deamplifiers); (2) increasing or decreasing transcription of the NFAT5/TonEBP (transcription up-regulators and down-regulators); (3) by increasing or decreasing the translation of NFAT5/TonEBP mRNA into protein (translation up-regulators and down-regulators); or (4) by increasing or decreasing the activity of NFAT5/TonEBP itself (agonists and antagonists).

To identify compounds that affect NFAT5/TonEBP at the DNA, RNA and protein levels, cells or organisms are contacted with a candidate compound and the corresponding change in NFAT5/TonEBP DNA, RNA or protein is assessed. For DNA amplifiers and deamplifiers, the amount of NFAT5/TonEBP DNA is measured, for those compounds that are transcription up-regulators and down-regulators the amount of NFAT5/TonEBP mRNA is determined; for translational up- and down-regulators, the amount of NFAT5/TonEBP polypeptides is measured. Compounds that are agonists or antagonists may be identified by contacting cells or organisms with the compound.

Modulators of NFAT5/TonEBP expression can be identified in a method where a cell is contacted with a candidate compound and the expression of NFAT5/TonEBP mRNA or protein in the cell is determined. The expression level of NFAT5/TonEBP mRNA or protein in the presence of the candidate compound is compared to NFAT5/TonEBP mRNA or protein levels in the absence of the candidate compound. The candidate compound can then be identified as a modulator of NFAT5/TonEBP mRNA or protein expression based upon this comparison. For example, when expression of NFAT5/TonEBP mRNA or protein is greater (i.e., statistically significant) in the presence of the candidate compound than in its absence, the candidate compound is identified as a stimulator of NFAT5/TonEBP mRNA or protein expression Alternatively, when expression of NFAT5/TonEBP mRNA or protein is less (statistically significant) in the presence of the candidate compound than in its absence, the candidate compound is identified as an inhibitor of NFAT5/TonEBP mRNA or protein expression. The level of NFAT5/TonEBP mRNA or protein expression in the cells can be determined by methods described for detecting NFAT5/TonEBP mRNA or protein.

Methods of making recombinant cells and expressing cellular proteins such as NFAT5/TonEBP are well known in the art. For an introduction to recombinant methods, see, Berger, Sambrook, and Ausubel, supra. Culture of mammalian cell lines and cultured cells from tissue or blood samples is well known in the art. Freshney (Culture of Animal Cells, a Manual of Basic Technique, Third Edition, Wiley-Liss, New York (1994)) and the references cited therein provides a general guide to the culture of animal cells. Culture of plant cells is described in Payne et al. (1992) Plant Cell and Tissue Culture in Liquid Systems, John Wiley & Sons, Inc., New York, N.Y. Additional information on cell culture, including prokaryotic cell culture, is found in Berger, Sambrook, and Ausubel, supra. Cell culture media are described in Atlas and Parks (eds) The Handbook of Microbiological Media (1993) CRC Press, Boca Raton, Fla. Additional information is found in commercial literature such as the Life Science Research Cell Culture catalogue (various editions) from Sigma-Aldrich, Inc. (St. Louis, Mo.) and, e.g., the Plant Culture Catalogue and supplement (1997) also from Sigma-Aldrich, Inc. (St. Louis, Mo.).

Many other assays for screening candidate or test compounds that bind to or modulate the activity of NFAT5/TonEBP or polypeptide or biologically active portion are available to those d skill in the art. Test compounds can be obtained using any of the numerous approaches in combinatorial library methods, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptides, while the other four approaches encompass peptide, non-peptide oligomer or small molecule libraries of compounds.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries are also optionally used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with α-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Berger and Kimmel, Guide to Molecular Cloning Techniques, Methods in Enzymology, volume 152, Academic Press, Inc., San Diego, Calif. (“Berger”), Sambrook, supra, and Ausubel, supra; peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan. 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

A “small molecule” refers to a composition that has a molecular weight of less than about 5 kD and more preferably less than about 4 kD, and most preferable less than 0.6 kD. Small molecules can be nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Cell-Free Assays

In one embodiment, a cell-free assay is provided which comprises contacting NFAT5/TonEBP or biologically-active fragment with a known compound that binds NFAT5/TonEBP to form an assay mixture, contacting the assay mixture with a test compound, and determining the ability of the test compound to interact with NFAT5/TonEBP, where determining the ability of the test compound to interact with NFAT5/TonEBP comprises determining the ability of the NFAT5/TonEBP to preferentially bind to or modulate the activity of a NFAT5/TonEBP target molecule.

The cell-free assays of the invention may be used with both soluble and membrane-bound forms of NFAT5/TonEBP. In the case of cell-free assays comprising the membrane-bound form, a solubilizing agent to maintain NFAT5/TonEBP in solution. Examples of such solubilizing agents include nonionic detergents such as n-octylglucoside, n-dodecylglucoside, n-dodecylmaltoside, octanoyl-N-methylglucamide, decanoyl-N-methylglucamide, TRITON® X-100 and others from the TRITON® series, THESIT®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

Immobilizing either NFAT5/TonEBP or its partner molecules can facilitate separation of complexed from uncomplexed forms of one or both of the proteins, as well as to accommodate high throughput assays. Binding of a test compound to NFAT5/TonEBP, or interaction of NFAT5/TonEBP with a target molecule in the presence and absence of a candidate compound, can be accomplished in any vessel suitable for containing the reactants, such as microtiter plates, test tubes, and micro-centrifuge tubes. A fusion protein can be provided that provides a domain that allows one or both of the proteins to be bound to a matrix. Examples of such fusion proteins are provided in Table 3 below. For example, GST-NFAT5/TonEBP fusion proteins or GST-target fusion proteins can be adsorbed onto glutathione sepharose beads (SIGMA Chemical, St. Louis, Mo.) or glutathione derivatized microtiter plates that are then combined with the test compound or the test compound and either the non-adsorbed target protein or NFAT5/TonEBP, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described. Alternatively, the complexes can be dissociated from the matrix, and the level of NFAT5/TonEBP binding or activity determined using standard techniques.

Other techniques for immobilizing proteins on matrices can also be used in screening assays. Either NFAT5/TonEBP or its target molecule can be immobilized using biotin-avidin or biotin-streptavidin systems. Biotinylation can be accomplished using many reagents, such as biotin-NHS (N-hydroxy-succinimide; PIERCE Chemicals, Rockford, Ill.), and immobilized in wells of streptavidin-coated 96 well plates (PIERCE Chemical). Alternatively, Abs reactive with NFAT5/TonEBP or target molecules, but which do not interfere with binding of the NFAT5/TonEBP to its target molecule, can be derivatized to the wells of the plate, and unbound target or NFAT5/TonEBP trapped in the wells by antibody conjugation. Methods for detecting such complexes, in addition to those described for the GST-immobilized complexes, include immunodetection of complexes using Abs reactive with NFAT5/TonEBP or its target, as well as enzyme-linked assays that rely on detecting an enzymatic activity associated with the NFAT5/TonEBP or target molecule.

Many other embodiments of such drug screening assays are available to those of skill in the art. For example, the following references provide multiple screening protocols which may be adapted for use with NFAT5/TonEBP: Sambrook et al. (1989) Molecular Cloning—A Laboratory Manual (2nd ed.) Vol. 1-3, Cold Spring Harbor Laboratory, Cold Spring Harbor Press, N.Y., (“Sambrook”); and Current Protocols in Molecular Biology, F. M. Ausubel et al., eds., Current Protocols, a joint venture between Greene Publishing Associates, Inc. and John Wiley & Sons, Inc., (e.g., current through 1999, e.g., at least through supplement 37) (“Ausubel”)), each of which are incorporated herein by reference in its entirety.

In one such embodiment, a screen based on the essential function of the NFAT5/TonEBP transcription factor in the mammalian osmotic stress response can involve using the DNA sequence to which this transcription factor binds to generate a reporter construct that is activated upon activation of the NFAT5/TonEBP transcription factor, which occurs in response to hypertonic stress. Such binding sites are known in the art and may be obtained in the following references, each of which is incorporated herein by reference in its entirety—Irarrazabal C E, Liu J C, Burg M B, Ferraris J D. ATM, a DNA damage-inducible kinase, contributes to activation by high NaCl of the transcription factor TonEBP/OREBP. Proc Natl Acad Sci USA. 2004 Jun. 8; 101(23):8809-14; Ko B C, Lam A K, Kapus A, Fan L, Chung S K, Chung S S. Fyn and p38 signaling are both required for maximal hypertonic activation of the osmotic response element-binding protein/tonicity-responsive enhancer-binding protein (OREBP/TonEBP). J Biol. Chem. 2002 Nov. 29; 277(48):46085-92; and Ferraris J D, Persaud P, Williams C K, Chen Y, Burg M B. cAMP-independent role of PKA in tonicity-induced transactivation of tonicity-responsive enhancer/osmotic response element-binding protein. Proc Natl Acad Sci USA. 2002 Dec. 24; 99(26):16800-5.

The reporter construct could then be used in cell-based assays for drugs that would inhibit the induction of reporter gene activity in response to hypertonic stress. Such an assay would not only identify drugs that may specifically target and inhibit the NFAT5/TonEBP transcription factor, but would also identify drugs that would target and inhibit any essential molecule that functions upstream of NFAT5/TonEBP in the osmotic stress response pathway.

In addition, any component of the osmotic stress response pathway could be used in such a screen. For example, a critical regulatory kinase whose function is essential to the activation of the pathway (e.g., for activation of the NFAT5/TonEBP transcription factor) could also be used in homogenous (i.e., non-cell based) in vitro assays to identify drugs that effect this pathway.

Agonists and Antagonists

“Antagonist” includes any molecule that partially or fully blocks, inhibits, or neutralizes a biological activity of endogenous NFAT5/TonEBP. Similarly, “agonist” includes any molecule that mimics a biological activity of endogenous NFAT5/TonEBP. Molecules that can act as agonists or antagonists include Abs or Ab fragments, fragments or variants of endogenous NFAT5/TonEBP, peptides, antisense oligonucleotides, small organic molecules, etc.

To assay for antagonists, NFAT5/TonEBP is added to, or expressed in, a cell along with the compound to be screened for a particular activity. If the compound inhibits the activity of interest in the presence of the NFAT5/TonEBP, that compound is an antagonist to the NFAT5/TonEBP; if NFAT5/TonEBP activity is enhanced, the compound is an agonist.

NFAT5/TonEBP-expressing cells can be easily identified using any of the disclosed methods. For example, antibodies that recognize the amino- or carboxy-terminus of human NFAT5/TonEBP can be used to screen candidate cells by immunoprecipitation, Western blots, and immunohistochemical techniques. Likewise, SEQ ID NOs: 2, 4, 6, 8 and 10 can be used to design primers and probes that can detect NFAT5/TonEBP mRNA in cells or samples from cells.

Any molecule that alters NFAT5/TonEBP cellular effects is a candidate antagonist or agonist. Screening techniques well known to those skilled in the art can identify these molecules. Examples of antagonists and agonists include: (1) small organic and inorganic compounds, (2) small peptides, (3) Abs and derivatives, (4) polypeptides closely related to NFAT5/TonEBP, (5) antisense DNA and RNA, (6) ribozymes, (7) triple DNA helices, (8) siRNAs and (9) nucleic acid aptamers.

Small molecules that bind to the NFAT5/TonEBP active site or other relevant part of the polypeptide and inhibit the biological activity of the NFAT5/TonEBP are antagonists. Examples of small molecule antagonists include small peptides, peptide-like molecules, preferably soluble, and synthetic non-peptidyl organic or inorganic compounds. These same molecules, if they enhance NFAT5/TonEBP activity, are examples of agonists.

Almost any antibody that affects NFAT5/TonEBPs function is a candidate antagonist, and occasionally, agonist. Examples of antibody antagonists include polyclonal, monoclonal, single-chain, anti-idiotypic, chimeric Abs, or humanized versions of such Abs or fragments. Abs may be from any species in which an immune response can be raised. Humanized Abs are also contemplated.

Alternatively, a potential antagonist or agonist may be a closely related protein, for example, a mutated form of the NFAT5/TonEBP that recognizes a NFAT5/TonEBP-interacting protein but imparts no effect, thereby competitively inhibiting NFAT5/TonEBP action. Alternatively, a mutated NFAT5/TonEBP may be constitutively activated and may act as an agonist.

NFAT5/TonEBP Polynucleotides

In various embodiments of the present invention, the methods for identifying modulatory compounds can comprise the utilization of a NFAT5/TonEBP polynucleotide. One aspect of the invention pertains to using isolated polynucleotides that encode NFAT5/TonEBP or biologically-active portions thereof, as well as fragments sufficient for use as hybridization probes to identify NFAT5/TonEBP-encoding nucleic acids (e.g., NFAT5/TonEBP mRNAs) and fragments for use as polymerase chain reaction (PCR) primers for the amplification and/or mutation of NFAT5/TonEBP molecules. A “nucleic acid molecule” or polynucleotide includes DNA molecules (e.g., cDNA or genomic DNA), RNA molecules (e.g., mRNA), analogs of the DNA or RNA generated using nucleotide analogs, and derivatives, fragments and homologues. The nucleic acid molecule may be single-stranded or double-stranded, but preferably comprises double-stranded DNA.

Probes

Probes are nucleic acid sequences of variable length, preferably between at least about 10 nucleotides (nt), 100 nt, or many (e.g., 6,000 nt) depending on the specific use. Probes are used to detect identical, similar, or complementary nucleic acid sequences. Longer length probes can be obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies. Probes are substantially purified oligonucleotides that will hybridize under stringent conditions to at least optimally 12, 25, 50, 100, 150, 200, 250, 300, 350 or 400 consecutive sense strand nucleotide sequence of SEQ ID NOs: 2, 4, 6, 8 and 10; or an anti-sense strand nucleotide sequence of SEQ ID NO: 2, 4, 6, 8 and 10; or of a naturally occurring mutant of SEQ ID NO: 2, 4, 6, 8 and 10.

The full- or partial-length native sequence NFAT5/TonEBP may be used to “pull out” similar (homologous) sequences, such as: (1) full-length or fragments of NFAT5/TonEBP cDNA from a cDNA library from any species (e.g., human, murine, feline, canine, bacterial, viral, retroviral, yeast), (2) from cells or tissues, (3) variants within a species, and (4) homologues and variants from other species. See, Ausubel and Sambrook, supra. To find related sequences that may encode related genes, the probe may be designed to encode unique sequences or degenerate sequences. Sequences may also be genomic sequences including promoters, enhancer elements and introns of native sequence NFAT5/TonEBP.

For example, NFAT5/TonEBP coding region in another species may be isolated using such probes. A probe of about 40 bases is designed, based on NFAT5/TonEBP, and made. To detect hybridizations, probes are labeled using, for example, radionuclides such as 32P or 35S, or enzymatic labels such as alkaline phosphatase coupled to the probe via avidin-biotin systems. Labeled probes are used to detect nucleic acids having a complementary sequence to that of NFAT5/TonEBP in libraries of cDNA, genomic DNA or mRNA of a desired species.

Such probes can be used as a part of a diagnostic test kit for identifying cells or tissues which misexpress a NFAT5/TonEBP, such as by measuring a level of a NFAT5/TonEBP in a sample of cells from a subject e.g., detecting NFAT5/TonEBP mRNA levels or determining whether a genomic NFAT5/TonEBP has been mutated or deleted.

Isolated Nucleic Acids

An isolated nucleic acid molecule or polynucleotide is separated from other nucleic acid molecules that are present in the natural source of the nucleic acid. Preferably, an isolated nucleic acid is free of sequences that naturally flank the nucleic acid (i.e., sequences located at the 5′- and 3′-termini of the nucleic acid) in the genomic DNA of the organism from which the nucleic acid is derived. For example, in various embodiments, isolated NFAT5/TonEBP molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an isolated nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having a nucleotide sequence of SEQ ID NOs: 2, 4, 6, 8 or 10, or a complement thereof, can be isolated using standard molecular biology techniques and the provided sequence information. Using all or a portion of the nucleic acid sequence of SEQ ID NO: 2, 4, 6, 8 or 10 as a hybridization probe, NFAT5/TonEBP molecules can be isolated using standard hybridization and cloning techniques. See, Ausubel and Sambrook, supra.

PCR amplification techniques can be used to amplify NFAT5/TonEBP using cDNA, mRNA or alternatively, genomic DNA, as a template and appropriate oligonucleotide primers. Such nucleic acids can be cloned into an appropriate vector and characterized by DNA sequence analysis. Furthermore, oligonucleotides corresponding to NFAT5/TonEBP sequences can be prepared by standard synthetic techniques, e.g., an automated DNA synthesizer.

Oligonucleotides

An oligonucleotide comprises a series of linked nucleotide residues, which oligonucleotide has a sufficient number of nucleotide bases to be used in a PCR reaction or other application. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOs: 2, 4, 6, 8 or 10, or a complement thereof. Oligonucleotides may be chemically synthesized and may also be used as probes.

Complementary Nucleic Acid Sequences and Binding

In another embodiment, an isolated nucleic acid molecule that can be used in the invention comprises a nucleic acid molecule that is a complement of the nucleotide sequence shown in SEQ ID NOs: 2, 4, 6, 8 or 10, or a portion of one of the nucleotide sequences (e.g., a fragment that can be used as a probe or primer or a fragment encoding a biologically-active portion of a NFAT5/TonEBP). A nucleic acid molecule that is complementary to the nucleotide sequence shown in SEQ ID NOs: 2, 4, 6, 8 or 10, is one that is sufficiently complementary to the nucleotide sequence shown in SEQ ID NOs: 2, 4, 6, 8 or 10, that it can hydrogen bond with little or no mismatches to the nucleotide sequence shown in SEQ ID NOs: 2, 4, 6, 8 or 10, thereby forming a stable duplex.

“Complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Nucleic acid fragments are at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full-length sequence. Fragments may be derived from any contiguous portion of a nucleic acid or amino acid sequence of choice.

Derivatives and Analogs

Derivatives are nucleic acid sequences or amino acid sequences formed from the native compounds either directly or by modification or partial substitution. Analogs are nucleic acid sequences or amino acid sequences that have a structure similar to, but not identical to, the native compound but differ from it in respect to certain components or side chains. Analogs may be synthetic or from a different evolutionary origin and may have a similar or opposite metabolic activity compared to wild type. Homologues are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, over a nucleic acid or amino acid sequence of identical size or when compared to an aligned sequence in which the alignment is done by a computer homology program known in the art, e.g., BLAST, or whose encoding nucleic acid is capable of hybridizing to the complement of a sequence encoding the aforementioned proteins under stringent, moderately stringent, or low stringent conditions. See, Ausubel, supra.

Homology

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NFAT5/TonEBP. Isoforms can be expressed in different tissues of the same organism as a result of, for example, alternative splicing of RNA. Without being bound to a particular theory, alternative splicing is though to be the source of homologous human NFAT5/TonEBP isoforms a, b and c and murine isoforms a and b. Alternatively, different genes can encode isoforms. In the invention, homologous nucleotide sequences include nucleotide sequences encoding for a NFAT5/TonEBP of species other than humans, including, but not limited to: vertebrates, and thus can include, e.g., frog, mouse, rat, rabbit, dog, cat, cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NFAT5/TonEBP, e.g., SEQ ID NOs: 1, 3, and 5. Homologous nucleic acid sequences include those nucleic acid sequences that encode conservative amino acid substitutions in SEQ ID NOs: 1, 3, 5, 7 or 9, as well as a polypeptide possessing NFAT5/TonEBP biological activity.

Open Reading Frames

The open reading frame (ORF) of a NFAT5/TonEBP gene encodes NFAT5/TonEBP. An ORF is a nucleotide sequence that has a start codon (ATG) and terminates with one of the three “stop” codons (TAA, TAG, or TGA). In this invention, however, an ORF may be any part of a coding sequence that may or may not comprise a start codon and a stop codon. To achieve a unique sequence, preferable NFAT5/TonEBP ORFs encode at least 50 amino acids.

NFAT5/TonEBP Hybridization

Homologues (i.e., nucleic acids encoding NFAT5/TonEBP derived from species other than human) or other related sequences (e.g., paralogs) can be obtained by low, moderate or high stringency hybridization with all or a portion of the particular human sequence as a probe using methods well known in the art for nucleic acid hybridization and cloning.

The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide which decreases DNA duplex stability. In general, the longer the probe, the higher the temperature required for proper annealing. A common approach is to vary the temperature: higher relative temperatures result in more stringent reaction conditions. See, Ausubel, supra.

To hybridize under “stringent conditions” describes hybridization protocols in which nucleotide sequences at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

“Stringent hybridization conditions” conditions enable a probe, primer or oligonucleotide to hybridize only to its target sequence. Stringent conditions are sequence-dependent and will differ. Stringent conditions comprise: (1) low ionic strength and high temperature washes (e.g., 15 mM sodium chloride, 1.5 mM sodium citrate, 0.1% sodium dodecyl sulfate at 50° C.); (2) a denaturing agent during hybridization (e.g., 50% (v/v) formamide, 0.1% bovine serum albumin, 0.1% Ficoll, 0.1% polyvinylpyrrolidone, 50 mM sodium phosphate buffer (pH 6.5; 750 mM sodium chloride, 75 mM sodium citrate at 42° C.); or (3) 50% formamide. Washes typically also comprise 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Preferably, the conditions are such that sequences at least about 65%, 70%, 75%, 85%, 90%, 95%, 98%, or 99% homologous to each other typically remain hybridized to each other. These conditions are presented as examples and are not meant to be limiting.

“Moderately stringent conditions” use washing solutions and hybridization conditions that are less stringent, such that a polynucleotide will hybridize to the entire, fragments, derivatives or analogs of SEQ ID NOs: 2, 4, 6, 8 or 10. See Sambrook, supra. One example comprises hybridization in 6×SSC, 5×Denhardt's solution, 0.5% SDS and 100 mg/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions are described in Sambrook, supra.

“Low stringent conditions” use washing solutions and hybridization conditions that are less stringent than those for moderate stringency, such that a polynucleotide will hybridize to the entire, fragments, derivatives or analogs of SEQ ID NOs: 2, 4, 6, 8 or 10. See Sambrook, supra. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency, such as those for cross-species hybridizations are described in Ausubel, supra.

NFAT5/TonEBP Nucleic Acid Variants

The invention further encompasses using nucleic acid molecules that differ from the nucleotide sequences shown in SEQ ID NOs: 2, 4, 6, 8 and 10 due to degeneracy of the genetic code and thus encode the same NFAT5/TonEBP as that encoded by the nucleotide sequences shown in SEQ ID NOs: 2, 4, 6, 8 and 10. An isolated nucleic acid molecule of the invention has a nucleotide sequence encoding a protein having an amino acid sequence shown in SEQ ID NOs: 1, 3, 5, 7 or 9.

Moreover, NFAT5/TonEBP from other species that have a nucleotide sequence that differs from the sequence of SEQ ID NOs: 2, 4, 6, 8 or 10, are contemplated. Nucleic acid molecules corresponding to natural allelic variants and homologues of the NFAT5/TonEBP cDNAs of the invention can be isolated based on their homology to the NFAT5/TonEBP of SEQ ID NOs: 2, 4, 6, 8 or 10 using cDNA-derived probes to hybridize to homologous NFAT5/TonEBP sequences under stringent conditions.

“NFAT5/TonEBP variant polynucleotide” or “NFAT5/TonEBP variant nucleic acid sequence” means a nucleic acid molecule which encodes an active NFAT5/TonEBP that (1) has at least about 80% nucleic acid sequence identity with a nucleotide acid sequence encoding a full-length native NFAT5/TonEBP, (2) a full-length native NFAT5/TonEBP lacking the signal peptide, (3) an extracellular domain of a NFAT5/TonEBP, with or without the signal peptide, or (4) any other fragment of a full-length NFAT5/TonEBP. Ordinarily, a NFAT5/TonEBP variant polynucleotide will have at least about 80%, 81%, 82%, 83%, 84%, 85%, 86%, 87%, 88%, 89%, 90%, 91%, 92%, 93%, 94%, 95%, 96%, 97%, 98% or 99% nucleic acid sequence identity and yet more preferably at least about 99% nucleic acid sequence identity with the nucleic acid sequence encoding a full-length native NFAT5/TonEBP. A NFAT5/TonEBP variant polynucleotide may encode full-length native NFAT5/TonEBP lacking the signal peptide, an extracellular domain of a NFAT5/TonEBP, with or without the signal sequence, or any other fragment of a full-length NFAT5/TonEBP. Variants do not encompass the native nucleotide sequence.

Ordinarily, NFAT5/TonEBP variant polynucleotides are at least about 30 nucleotides in length, often at least about 60, 90, 120, 150, 180, 210, 240, 270, 300, 450, 600 nucleotides in length, more often at least about 900 nucleotides in length, or more.

“Percent (%) nucleic acid sequence identity” with respect to NFAT5/TonEBP-encoding nucleic acid sequences identified herein is defined as the percentage of nucleotides in a candidate sequence that are identical with the nucleotides in the NFAT5/TonEBP sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:
% nucleic acid sequence identity=W/Z·100
where W is the number of nucleotides cored as identical matches by the sequence alignment program's or algorithm's alignment of C and D and Z is the total number of nucleotides in D.

When the length of nucleic acid sequence C is not equal to the length of nucleic acid sequence D, the % nucleic acid sequence identity of C to D will not equal the % nucleic acid sequence identity of D to C.

In addition to naturally-occurring allelic variants of NFAT5/TonEBP, changes can be introduced by mutation into SEQ ID NOs: 2, 4, 6, 8 or 10 that incur alterations in the amino acid sequences of the encoded NFAT5/TonEBP that do not alter NFAT5/TonEBP function. For example, nucleotide substitutions leading to amino acid substitutions at “non-essential” amino acid residues can be made in the sequence of SEQ ID NOs: 1, 3, 5, 7 or 9. A “non-essential” amino acid residue is a residue that can be altered from the wild-type sequences of the NFAT5/TonEBP without altering their biological activity, whereas an “essential” amino acid residue is required for such biological activity. For example, amino acid residues that are conserved among the NFAT5/TonEBP of the invention are predicted to be particularly non-amenable to alteration. Amino acids for which conservative substitutions can be made are well known in the art.

Useful conservative substitutions are shown in Table 1. Conservative substitutions whereby an amino acid of one class is replaced with another amino acid of the same type fall within the scope of the subject invention so long as the substitution does not materially alter the biological activity of the compound. The invention can use mutant or variant NFAT5/TonEBP, any of which bases may be changed from the corresponding base shown in Table 2 while still encoding a polypeptide that maintains the activities and physiological functions of the NFAT5/TonEBP fragment, or a fragment of such a nucleic acid. If such substitutions result in a change in biological activity, then more substantial changes, indicated in Table 2 as examples are introduced and the products screened for NFAT5/TonEBP polypeptide biological activity.

TABLE 1
Preferred Amino Acid Substitutions

| Original | | Preferred |
|---|---|---|
| residue | Exemplary substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, Norleucine | Leu |

Non-conservative substitutions that affect (1) the structure of the polypeptide backbone, such as a β-sheet or α-helical conformation, (2) the charge or (3) hydrophobicity, or (4) the bulk of the side chain of the target site can modify NFAT5/TonEBP polypeptide function or immunological identity. Residues are divided into groups based on common side-chain properties as denoted in Table 2. Non-conservative substitutions entail exchanging a member of one of these classes for another class. Substitutions may be introduced into conservative substitution sites or more preferably into non-conserved sites.

TABLE 2
Amino acid classes

| Class | Amino acids |
|---|---|
| hydrophobic | Norleucine, Met, Ala, Val, Leu, Ile |
| neutral hydrophilic | Cys, Ser, Thr |
| acidic | Asp, Glu |
| basic | Asn, Gln, His, Lys, Arg |
| disrupt chain conformation | Gly, Pro |
| aromatic | Trp, Tyr, Phe |

The variant polypeptides can be made using methods known in the art such as oligonucleotide-mediated (site-directed) mutagenesis, alanine scanning and PCR mutagenesis. Site-directed mutagenesis, cassette mutagenesis, restriction selection mutagenesis or other known techniques can be performed on the cloned DNA to produce the NFAT5/TonEBP variant DNA. See, e.g., Ausubel and Sambrook, supra.

In one embodiment, the isolated nucleic acid molecule comprises a nucleotide sequence encoding a protein, wherein the protein comprises an amino acid sequence at least about 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to SEQ ID NOs: 1, 3, 5, 7 or 9.

NFAT5/TonEBP Polypeptides

In yet another aspect of the present invention, the cells can comprise a NFAT5/TonEBP variant or fragment protein. An NFAT5/TonEBP polypeptide includes the amino acid sequence of NFAT5/TonEBP whose sequences are provided in SEQ ID NOs: 1, 3, 5, 7 or 9. The invention also includes a mutant or variant protein any of whose residues may be changed from the corresponding residues shown in SEQ ID NOs: 1, 3, 5, 7 or 9, while still encoding a protein that maintains its NFAT5/TonEBP activities and physiological functions, or a functional fragment thereof.

In general, an NFAT5/TonEBP polypeptide variant preserves NFAT5/TonEBP-like function and includes any variant in which residues at a particular position in the sequence have been substituted by other amino acids, and further includes the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

An active NFAT5/TonEBP polypeptide or NFAT5/TonEBP polypeptide fragment retains a biological and/or an immunological activity similar, but not necessarily identical, to an activity of a naturally-occurring (wild-type) NFAT5/TonEBP polypeptide of the invention, including mature forms. A particular biological assay, with or without dose dependency, can be used to determine NFAT5/TonEBP activity. A nucleic acid fragment encoding a biologically-active portion of NFAT5/TonEBP can be prepared by isolating a portion of SEQ ID NOs: 2, 4, 6, 8 or 10 that encodes a polypeptide having a NFAT5/TonEBP biological activity (the biological activities of the NFAT5/TonEBP are described below), expressing the encoded portion of NFAT5/TonEBP (e.g., by recombinant expression in vitro) and assessing the activity of the encoded portion of NFAT5/TonEBP. Immunological activity refers to the ability to induce the production of an antibody against an antigenic epitope possessed by a native NFAT5/TonEBP; biological activity refers to a function, either inhibitory or stimulatory, caused by a native NFAT5/TonEBP that excludes immunological activity.

“NFAT5/TonEBP polypeptide variant” means an active NFAT5/TonEBP polypeptide having at least: (1) about 80% amino acid sequence identity with a full-length native sequence NFAT5/TonEBP polypeptide sequence, (2) a NFAT5/TonEBP polypeptide sequence lacking the signal peptide, (3) an extracellular domain of a NFAT5/TonEBP polypeptide, with or without the signal peptide, or (4) any other fragment of a full-length NFAT5/TonEBP polypeptide sequence. For example, NFAT5/TonEBP polypeptide variants include NFAT5/TonEBP polypeptides wherein one or more amino acid residues are added or deleted at the N- or C-terminus of the full-length native amino acid sequence. A NFAT5/TonEBP polypeptide variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, 98% or 99% amino acid sequence identity with a full-length native sequence NFAT5/TonEBP polypeptide sequence. A NFAT5/TonEBP polypeptide variant may have a sequence lacking the signal peptide, an extracellular domain of a NFAT5/TonEBP polypeptide, with or without the signal peptide, or any other fragment of a full-length NFAT5/TonEBP polypeptide sequence. Ordinarily, NFAT5/TonEBP variant polypeptides are at least about 10 amino acids in length, often at least about 20 amino acids in length, more often at least about 30, 40, 50, 60, 70, 80, 90, 100, 150, 200, or 300 amino acids in length, or more.

“Percent (%) amino acid sequence identity” is defined as the percentage of amino acid residues that are identical with amino acid residues in the disclosed NFAT5/TonEBP polypeptide sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Often publicly available computer software such as BLAST, BLAST2, ALIGN2 or Megalign (DNASTAR) software is used to align peptide sequences. Those skilled in the art can determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:
% amino acid sequence identity=X/Y·100
where X is the number of amino acid residues scored as identical matches by the sequence alignment program's or algorithm's alignment of A and B and Y is the total number of amino acid residues in B.

If the length of amino acid sequence A is not equal to the length of amino acid sequence B, the % amino acid sequence identity of A to B will not equal the % amino acid sequence identity of B to A.

Biologically active portions of NFAT5/TonEBP include peptides comprising amino acid sequences sufficiently homologous to or derived from the amino acid sequences of the NFAT5/TonEBP (SEQ ID NOs: 1, 3, 5, 7 or 9) that include fewer amino acids than the full-length NFAT5/TonEBP, and exhibit at least one activity of a NFAT5/TonEBP. Biologically active portions comprise a domain or motif with at least one activity of native NFAT5/TonEBP. A biologically active portion of a NFAT5/TonEBP can be a polypeptide that is, for example, 10, 25, 50, 100 or more amino acid residues in length. Other biologically active portions, in which other regions of the protein are deleted, can be prepared by recombinant techniques and evaluated for one or more of the functional activities of a native NFAT5/TonEBP.

Biologically active portions of NFAT5/TonEBP may have an amino acid sequence shown in SEQ ID NOs: 1, 3, 5, 7 or 9, or substantially homologous to SEQ ID NOs: 1, 3, 5, 7 or 9, and retains the functional activity of the protein of SEQ ID NOs: 1, 3, 5, 7 or 9, yet differs in amino acid sequence due to natural allelic variation or mutagenesis. Other biologically active NFAT5/TonEBP may comprise an amino acid sequence at least about 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to the amino acid sequence of SEQ ID NOs: 1, 3, 5, 7 or 9, and retains the functional activity of native NFAT5/TonEBP.

Fusion polypeptides are useful in expression studies, cell-localization, bioassays, and NFAT5/TonEBP purification. A NFAT5/TonEBP “chimeric protein” or “fusion protein” comprises NFAT5/TonEBP fused to a non-NFAT5/TonEBP polypeptide. A non-NFAT5/TonEBP polypeptide is not substantially homologous to NFAT5/TonEBP (SEQ ID NOs: 1, 3, 5, 7 or 9). A NFAT5/TonEBP fusion protein may include any portion to the entire NFAT5/TonEBP, including any number of the biologically active portions. NFAT5/TonEBP may be fused to the C-terminus of the GST (glutathione S-transferase) sequences. Such fusion proteins facilitate the purification of recombinant NFAT5/TonEBP. In certain host cells, (e.g., mammalian), heterologous signal sequences fusions may ameliorate NFAT5/TonEBP expression and/or secretion. Additional exemplary fusions are presented in Table 3.

Other fusion partners can adapt NFAT5/TonEBP therapeutically. Fusions with members of the immunoglobulin (Ig) protein family are useful in therapies that inhibit NFAT5/TonEBP ligand or substrate interactions, consequently suppressing NFAT5/TonEBP-mediated signal transduction in vivo. NFAT5/TonEBP-Ig fusion polypeptides can also be used as immunogens to produce anti-NFAT5/TonEBP Abs in a subject, to purify NFAT5/TonEBP ligands, and to screen for molecules that inhibit interactions of NFAT5/TonEBP with other molecules.

Fusion proteins can be easily created using recombinant methods. A nucleic acid encoding NFAT5/TonEBP can be fused in-frame with a non-NFAT5/TonEBP encoding nucleic acid, to the NFAT5/TonEBP NH₂— or COO-terminus, or internally. Fusion genes may also be synthesized by conventional techniques, including automated DNA synthesizers. PCR amplification using anchor primers that give rise to complementary overhangs between two consecutive gene fragments that can subsequently be annealed and reamplified to generate a chimeric gene sequence is also useful. See Ausubel, supra. Many vectors are commercially available that facilitate sub-cloning NFAT5/TonEBP in-frame to a fusion moiety.

TABLE 3
Useful non-NFAT5lTonEBP fusion polypeptides

| Reporter | in vitro | in vivo | Notes |
|---|---|---|---|
| Human growth | Radioimmuno- | none | Expensive, insensitive, |
| hormone (hGH) | assay | | narrow linear range. |
| β-glucuronidase | Colorimetric, | colorimetric | sensitive, broad linear range, |
| (GUS) | fluorescent, or | (histochemical | non-isotopic. |
| | chemiluminescent | staining | |
| | | with X-gluc) | |
| Green fluorescent | Fluorescent | fluorescent | can be used in live cells; |
| protein (GFP) and | | | resists photo-bleaching |
| related molecules | | | |
| (RFP, BFP, | | | |
| NFAT5/TonEBP, | | | |
| etc.) | | | |
| Luciferase (firefly) | bioluminescent | Bio- | protein is unstable, difficult to |
| | | luminescent | reproduce, signal is brief |
| Chloramphenicol | Chromatography, | none | Expensive radioactive |
| acetyltransferase | differential | | substrates, time-consuming, |
| (CAT) | extraction, | | insensitive, narrow linear |
| | fluorescent, or | | range |
| | immunoassay | | |
| β-galactosidase | colorimetric, | colorimetric | sensitive, broad linear range; |
| | fluorescence, | (histochemical | some cells have high |
| | chemiluminscence | staining | endogenous activity |
| | | with X-gal), | |
| | | bio- | |
| | | luminescent | |
| | | in live cells | |
| Secrete alkaline | colorimetric, | none | Chemiluminscence assay is |
| phosphatase (SEAP) | bioluminescent, | | sensitive and broad linear |
| | chemiluminescent | | range; some cells have |
| | | | endogenous akaline |
| | | | phosphatase activity |

The assay for cell growth or viability can comprise an assay selected from the group consisting of a cell uptake assay, cell binding assay, growth inhibition assay and any combination thereof. A “cell uptake assay” comprises measurement of the uptake of a membrane-permeable chemical probe and its intracellular conversion to a fluorescent or colored compound by viable, metabolically active cells relevant to a control. Such an assay also comprises measurement of the concentration of total cellular protein, DNA or RNA; incorporation of a membrane-permeable radio-labeled or fluorescent biochemical precursors by viable, metabolically active cells relevant to a control. Such an assay also comprises measurement of the release of a biochemical marker by injured, dead or dying cells relevant to a control. A “cell binding assay” comprises measurement of the uptake and binding of a membrane-impermeable fluorescent or colored nucleic acid stain or dye by injured, dead or dying cells. A “growth inhibition assay” comprises measurement of protein concentration, which reflects cell growth or viability, using a protein-binding dye and spectrophotometric quantitation relevant to a control. A. Monks et al. Feasibility of a high-flux anticancer drug screen using a diverse panel of cultured human tumor cell lines. J Natl Cancer Inst. 1991 Jun. 5; 83(11):757-66. Such an assay also comprises measurement of cell number (i.e., a cell count) relevant to a control. Other cell growth and viability assays are known b those of skill in the art.

In another aspect of the present invention, a method is provided for identifying a candidate anti-cancer or immunosuppressive compound, the method comprising (a) performing a structure based drug design using a three dimensional structure determined for a crystal of NFAT5/TonEBP. The three dimensional structure can comprise atomic coordinates of Protein Data Bank Accession No. 1IMH, incorporated herein by reference in its entirety.

Crystal Structure Docking

According to an aspect of the present invention, crystalline NFAT5/TonEBP protein can be used to determine the ability of a compound of the present invention to bind to an NFAT5/TonEBP protein in a manner predicted by a structure-based drug design method of the present invention. In various aspects of the present invention, a NFAT5/TonEBP protein crystal can be soaked in a solution containing a chemical compound of the present invention. Binding of the chemical compound to the crystal is then determined by methods standard in the art.

One aspect of the present invention is a therapeutic composition. A therapeutic composition of the present invention comprises one or more therapeutic compounds. In one aspect, a therapeutic composition is provided that is capable of inhibiting osmotic stress that involves an NFAT5/TonEBP protein. For example, a therapeutic composition of the present invention can inhibit (i.e., prevent, block) binding of an NFAT5/TonEBP protein on a cell to a molecule by interfering with the, e.g., DNA binding site of the NFAT5/TonEBP protein. As used herein, the term “binding site” refers to the region of a NFAT5/TonEBP protein to which a ligand or substrate specifically binds. In one aspect of the present invention, a method is provided for inhibiting, e.g., cancer or inflammation, in a subject comprising administering to the subject in need thereof a therapeutically effective amount of a therapeutic composition of the present invention.

Suitable inhibitory compounds of the present invention are compounds that interact directly with an NFAT5/TonEBP protein thereby inhibiting the binding of an NFAT5/TonEBP ligand or substrate, e.g., DNA, to an NFAT5/TonEBP protein, by blocking the ligand or substrate binding site of an NFAT5/TonEBP protein (referred to herein as substrate analogs). An NFAT5/TonEBP substrate analog refers to a compound that interacts with (e.g., binds to, associates with, modifies) the binding site of an NFAT5/TonEBP protein. An NFAT5/TonEBP substrate analog can, for example, comprise a chemical compound that mimics the Rel- or Rel-like DNA binding domain or other ligand or substrate binding site of an NFAT5/TonEBP protein.

According to the present invention, suitable therapeutic compounds of the present invention include peptides or other organic molecules, and inorganic molecules. Suitable organic molecules include small organic molecules. In various aspects, a therapeutic compound of the present invention is not harmful (e.g., toxic) to an animal when such compound is administered to an animal. Peptides refer to a class of compounds that is small in molecular weight and yields two or more amino acids upon hydrolysis. A polypeptide is comprised of two or more peptides. As used herein, a protein is comprised of one or more polypeptides. Suitable therapeutic compounds to design include peptides composed of “L” and/or “D” amino acids that are configured as normal or retroinverso peptides, peptidomimetic compounds, small organic molecules, or homo- or hetero-polymers thereof, in linear or branched configurations.

Therapeutic compounds of the present invention can be designed using structure based drug design. Structure based drug design refers to the use of computer simulation to predict a conformation of a peptide, polypeptide, protein, or conformational interaction between a peptide or polypeptide, and a therapeutic compound. In the present teachings, knowledge of the three dimensional structure of the NFAT5/TonEBP protein provides one of skill in the art the ability to design a therapeutic compound that binds to NFAT5/TonEBP proteins, is stable and results in inhibition of a biological response. The three dimensional structure of NFAT5/TonEBP protein isoform a (SEQ ID NO: 1) is disclosed in the Protein Data Bank as Accession No. 1IMH. For example, and without limitation, knowledge of the three dimensional structure of the DNA binding site provides to a skilled artisan the ability to design an analog of a ligand, substrate or polynucleotide which can function as an inhibitor of an NFAT5/TonEBP protein.

Suitable structures and models useful for structure-based drug design include molecular replacement. Methods of molecular replacement are generally known by those of skill in the art and are performed in a software program including, for example, X-PLOR available from Accelerys (San Diego, Calf.). In various aspects of the invention, the three dimensional structure of NFAT5/TonEBP protein useful in a method of molecular replacement according to the present invention has an amino acid sequence that is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the search structure (e.g., NFAT5/TonEBP; SEQ ID NO: 1), when the two amino acid sequences are compared using an alignment program such as BLAST (supra). Models of target structures to use in a method of structure-based drug design include models produced by any modeling method disclosed herein, such as, for example, molecular replacement and fold recognition related methods which are well understood in the art. In some aspects of the present invention, structure based drug design can be applied to a structure of NFAT5/TonEBP in complex with a ligand or substrate, and to a model of a target NFAT5/TonEBP structure.

One embodiment of the present invention is a method for designing a drug which interferes with an activity of an NFAT5/TonEBP protein. In various configurations, the method comprises providing a three-dimensional structure of a NFAT5/TonEBP protein comprising at least one ligand of the protein, and designing a chemical compound which is predicted to bind to the protein. The designing can comprise using physical models, such as, for example, ball-and-stick representations of atoms and bonds, or on a digital computer equipped with molecular modeling software. In some configurations, these methods can further include synthesizing the chemical compound, and evaluating the chemical compound for ability to interfere with an activity of the NFAT5/TonEBP protein.

Suitable three dimensional structures of a NFAT5/TonEBP protein and models to use with the present method are disclosed herein. According to the present invention, designing a compound can include creating a new chemical compound or searching databases of libraries of known compounds (e.g., a compound listed in a computational screening database containing three dimensional structures of known compounds). Designing can also include simulating chemical compounds having substitute moieties at certain structural features. In some configurations, designing can include selecting a chemical compound based on a known function of the compound. In some configurations designing can comprise computational screening of one or more databases of compounds in which three dimensional structures of the compounds are known. In these configurations, a candidate compound can be interacted virtually (e.g., docked, aligned, matched, interfaced) with the three dimensional structure of a NFAT5/TonEBP protein by computer equipped with software such as, for example, the AutoDock software package, (The Scripps Research Institute, La Jolla, Calif.) or described by Humblet and Dunbar, Animal Reports in Medicinal Chemistry, vol. 28, pp. 275-283, 1993, M Venuti, ed., Academic Press. Methods for synthesizing candidate chemical compounds are known to those of skill in the art.

Various other methods of structure-based drug design are disclosed in references such as Maulik et al., 1997, Molecular Biotechnology: Therapeutic Applications and Strategies, Wiley-Liss, Inc., which is incorporated herein by reference in its entirety. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional structures and small fragment probes, followed by linking together of favorable probe sites.

In one aspect, a chemical compound of the present invention that binds to the DNA binding site can be a chemical compound having chemical and/or stereochemical complementarity with an NFAT5/TonEBP protein, e.g., a Rel- or Rel-like DNA binding site. In some configurations, a chemical compound that binds to the selected binding site can associate with an affinity of at least about 10⁻⁶ M, at least about 10⁻⁷ M, or at least about 10⁻⁸ M.

Drug design strategies as specifically described above with regard to residues and regions of the ligand-complexed NFAT5/TonEBP crystal can be similarly applied to other NFAT5/TonEBP protein structures. One of ordinary skill in the art, using the art recognized modeling programs and drug design methods, many of which are described herein, can modify the NFAT5/TonEBP protein design strategy according to differences in amino acid sequence. For example, this strategy can be used to design compounds which regulate osmotic stress in other NFAT5/TonEBP proteins or NFAT5/TonEBP variants. In addition, one of skill in the art can use lead compound structures derived from one NFAT5/TonEBP protein and take into account differences in amino acid residues in other NFAT5/TonEBP proteins or variants.

In the present method of structure-based drug design, it is not necessary to align a candidate chemical compound (i.e., a chemical compound being analyzed in, for example, a computational screening method of the present invention) to each residue in a target site. Suitable candidate chemical compounds can align to a subset of residues described for a target site. In some configurations of the present invention, a candidate chemical compound can comprise a conformation that promotes the formation of covalent or noncovalent crosslinking between the target site and the candidate chemical compound. In certain aspects of the invention, a candidate chemical compound can bind to a surface adjacent to a target site to provide an additional site of interaction in a complex. For example, when designing an antagonist (i.e., a chemical compound that inhibits the binding of a ligand to an NFAT5/TonEBP protein by blocking a binding site or interface), the antagonist can be designed to bind with sufficient affinity to the binding site or to substantially prohibit a ligand (i.e., a molecule that specifically binds to the target site) from binding to a target area. It will be appreciated by one of skill in the art that it is not necessary that the complementarity between a candidate chemical compound and a target site extend over all residues specified here.

In various aspects, the design of a chemical compound possessing stereochemical complementarity can be accomplished by means of techniques that optimize, chemically or geometrically, the “fit” between a chemical compound and a target site. Such techniques are disclosed by, for example, Sheridan and Venkataraghavan, Acc. Chem. Res., vol. 20, p. 322, 1987: Goodford, J. Med. Chem., vol. 27, p. 557, 1984; Beddell, Chem. Soc. Reviews, vol. 279, 1985; Hol, Angew. Chem., vol. 25, p. 767, 1986; and Verlinde and Hol, Structure, vol. 2, p. 577, 1994, each of which is incorporated by this reference herein in their entirety.

Some embodiments of the present invention for structure-based drug design comprise methods of identifying a chemical compound that complements the shape of an NFAT5/TonEBP protein or a structure that is related to an NFAT5/TonEBP protein. Such method is referred to herein as a “geometric approach”. In a geometric approach of the present invention, the number of internal degrees of freedom (and the corresponding local minima in the molecular conformation space) can be reduced by considering only the geometric (hard-sphere) interactions of two rigid bodies, where one body (the active site) contains “pockets” or “grooves” that form binding sites for the second body (the complementing molecule, such as a ligand or substrate).

The geometric approach is described by Kuntz et al., J. Mol. Biol., vol. 161, p. 269, 1982, which is incorporated by this reference herein in its entirety. The algorithm for chemical compound design can be implemented using a software program such as AutoDock, available from The Scripps Research Institute (La Jolla, Calif.). One or more extant databases of crystallographic data (e.g., the Cambridge Structural Database System maintained by University Chemical Laboratory, Cambridge University, Lensfield Road, Cambridge CB2 IEW, U.K. or the Protein Data Bank maintained by Rutgers University) can then be searched for chemical compounds that approximate the shape thus defined. Chemical compounds identified by the geometric approach can be modified to satisfy criteria associated with chemical complementarity, such as hydrogen bonding, ionic interactions or Van der Waals interactions.

The crystal docking method may further comprise (b) contacting the candidate inhibitor with a cell comprising NFAT5/TonEBP or a variant or fragment thereof; and (c) detecting inhibition of at least one activity of NFAT5/TonEBP, variant or fragment thereof. Alternatively, the method may further comprise (b) contacting the candidate inhibitor with a cell comprising NFAT5/TonEBP or a variant or fragment thereof; (c) culturing the cell under conditions of hypertonic stress; (d) contacting the cell with the candidate compound; and (e) assaying the cell for cell growth or viability. In various aspects, the assay for cell growth or viability can comprise an assay selected from the group consisting of a cell uptake assay, cell binding assay, growth inhibition assay and any combination thereof.

In yet another aspect of the present invention, a method is provided for treating a cancer or an autoimmune disease in a subject, the method comprising administering to a subject in need thereof a therapeutically effective amount of an inhibitor of NFAT5/TonEBP. In various aspects, the inhibitor is selected by (a) culturing a first cell and a second cell under conditions of hypertonic stress; (b) contacting the first cell with a test agent; and (c) assaying the first cell and the second cell for cell growth or viability, wherein a decrease in cell growth or viability in the first cell relative to the second cell indicates that the test agent is an inhibitor of NFAT5/TonEBP. In various aspects, the first and second cell can comprise an NFAT5/TonEBP polynucleotide or polypeptide as described above. In various other aspects, the first and second cell can comprise a NFAT5/TonEBP variant polynucleotide as described above. In yet another aspect, the first and second cell can comprise a NFAT5/TonEBP variant or fragment protein as described above. The assay for cell growth or viability can comprise an assay selected from the group consisting of a cell uptake assay, cell binding assay, growth inhibition assay and any combination thereof as described above.

Prophylactic Methods

The invention provides a method for preventing, in a subject, a disease or condition associated with an aberrant NFAT5/TonEBP expression or activity, by administering an agent that modulates NFAT5/TonEBP expression or at least one NFAT5/TonEBP activity. Subjects at risk for a disease that is caused or contributed to by aberrant NFAT5/TonEBP expression or activity can be identified by, for example, any or a combination of diagnostic or prognostic assays. Administration of a prophylactic agent can occur prior to the manifestation of symptoms characteristic of the NFAT5/TonEBP aberrancy, such that a disease or disorder is prevented or, alternatively, delayed in its progression. Depending on the type of NFAT5/TonEBP aberrancy, for example, a NFAT5/TonEBP agonist or NFAT5/TonEBP antagonist can be used to treat the subject. The appropriate agent can be determined based on screening assays.

Therapeutic Methods

Another aspect of the invention pertains to methods of modulating NFAT5/TonEBP expression or activity for therapeutic purposes. The modulatory method of the invention involves contacting a cell with an agent that modulates one or more of the activities of NFAT5/TonEBP activity associated with the cell. An agent that modulates NFAT5/TonEBP activity can be a nucleic acid or a protein, a naturally occurring cognate ligand of NFAT5/TonEBP, a peptide, a NFAT5/TonEBP peptidomimetic, or other small molecule. The agent may stimulate NFAT5/TonEBP activity Examples of such stimulatory agents include active NFAT5/TonEBP and a NFAT5/TonEBP nucleic acid molecule that has been introduced into the cell. In another embodiment, the agent inhibits NFAT5/TonEBP activity. Examples of inhibitory agents include antisense NFAT5/TonEBP nucleic acids and anti-NFAT5/TonEBP Abs. Modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NFAT5/TonEBP or nucleic acid molecule. In one embodiment, the method involves administering an agent (e.g., an agent identified by a screening assay), or combination of agents that modulates (e.g., up-regulates or down-regulates) NFAT5/TonEBP expression or activity. In another embodiment, the method involves administering a NFAT5/TonEBP or nucleic acid molecule as therapy to compensate for reduced or aberrant NFAT5/TonEBP expression or activity.

In another aspect of the invention, proteases would be released within the microenvironment of the tumor, either in combination with other therapeutic agents or alone, to produce osmotic stress on the tumor cells which will ultimately result in tumor cell death. Such proteases would include (but are not limited to): caspases, aspariginyl endopeptidase, cathepsins, matrix metalloproteinases. The resultant tumor cell death in response to osmotic stress will in turn cause the release of more protease, thus resulting in a cycle of protease activity and cell death that will keep tumor growth in check. In another embodiment, the proteases introduced to and released into the microenvironment of the tumor will be biologically engineered to enable release of the proteases at a pre-determined region of the tumor. Such bioengineering is well known in the art, and includes methods and molecules in the following non-exhaustive list: “caging” the protease molecules within a chemically or photo-labile molecule, in which the chemically or photo-labile molecule can be triggered to disintegrate or otherwise release the proteases using a stimulus specific to the molecule used as the “cage”. In another embodiment, the protease molecules are genetically or otherwise biologically engineered to be expressed linked to an inactivating molecule. Such techniques are also well known in the art, and could include attaching through an inert nucleotide linker, such linker capable of being enzymatically or photolytically cleaved once the linked proteases have entered the microenvironment of the tumor.

Stimulation of NFAT5/TonEBP activity is desirable in situations in which NFAT5/TonEBP is abnormally down-regulated and/or in which increased NFAT5/TonEBP activity is likely to have a beneficial effect.

Determination of the Biological Effect of the Therapeutic

Suitable in vitro or in vivo assays can be performed to determine the effect of a specific therapeutic and whether its administration is indicated for treatment of the affected tissue. In various specific embodiments, in vitro assays may be performed with representative cells of the type(s) involved in the patient's disorder, to determine if a given therapeutic exerts the desired effect upon the cell type(s). Modalities for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of Compositions

NFAT5/TonEBP nucleic acids and proteins are useful in potential prophylactic and therapeutic applications implicated in a variety of disorders including, but not limited to cancers and autoimmune diseases. As an example, a cDNA encoding NFAT5/TonEBP may be useful in gene therapy, and the protein may be useful when administered to a subject in need thereof. By way of non-limiting example, the compositions of the invention will have efficacy for treatment of patients suffering from cancer and autoimmune diseases.

NFAT5/TonEBP nucleic acids, or fragments thereof, may also be useful in diagnostic applications, wherein the presence or amount of the nucleic acid or the protein is to be assessed. A further use could be as an anti-bacterial molecule (i.e., some peptides have been found to possess anti-bacterial properties). These materials are further useful in the generation of Abs that immunospecifically bind to the novel substances of the invention for use in therapeutic or diagnostic methods.

In another aspect of the present invention, the inhibitor is selected by: (a) performing a structure based drug design using a three dimensional structure determined for a crystal of NFAT5/TonEBP. The three dimensional structure can comprise atomic coordinates of Protein Data Bank Accession No. 1IMH as described above. The method may further comprise (b) contacting the candidate inhibitor with a cell comprising NFAT5/TonEBP or a variant or fragment thereof; and (c) detecting inhibition of at least one activity of NFAT5/TonEBP, variant or fragment thereof. Alternatively, the method may further comprise (b) contacting the candidate inhibitor with a cell comprising NFAT5/TonEBP or a variant or fragment thereof; (c) culturing the cell under conditions of hypertonic stress; (d) contacting the cell with the candidate compound; and (e) assaying the cell for cell growth or viability. In various aspects, the assay for cell growth or viability can comprise an assay selected from the group consisting of a cell uptake assay, cell binding assay, growth inhibition assay and any combination thereof. In various aspects, the cell can comprise a NFAT5/TonEBP variant polynucleotide or NFAT5/TonEBP variant or fragment protein. In various aspects, the inhibitor is an antibody directed against NFAT5/TonEBP, preferably a monoclonal antibody.

Anti-NFAT5/TonEBP Abs

The invention makes use of Abs and antibody fragments, such as F_{ab} or (F_{ab})₂, that bind immunospecifically to any NFAT5/TonEBP epitopes. “Antibody” (Ab) comprises single Abs directed against NFAT5/TonEBP (anti-NFAT5/TonEBP Ab; including agonist, antagonist, and neutralizing Abs), anti-NFAT5/TonEBP Ab compositions with poly-epitope specificity, single chain anti-NFAT5/TonEBP Abs, and fragments of anti-NFAT5/TonEBP Abs. A “monoclonal antibody” is obtained from a population of substantially homogeneous Abs, i.e., the individual Abs comprising the population are identical except for possible naturally-occurring mutations that may be present in minor amounts. Exemplary Abs include polyclonal (pAb), monoclonal (mAb), humanized, bi-specific (bsAb), and heteroconjugate Abs. Antibodies can be produced by any known method in the art or obtained commercially.

Monovalent Abs

The Abs may be monovalent Abs that consequently do not cross-link with each other. For example, one method involves recombinant expression of Ig light chain and modified heavy chain. Heavy chain truncations generally at any point in the F_{c} region will prevent heavy chain cross-linking. Alternatively, the relevant cysteine residues are substituted with another amino acid residue or are deleted, preventing crosslinking. In vitro methods are also suitable for preparing monovalent Abs. Abs can be digested to produce fragments, such as F_{ab} fragments.

Humanized and Human Abs

Anti-NFAT5/TonEBP Abs may further comprise humanized or human Abs. Humanized forms of non-human Abs are chimeric Igs, Ig chains or fragments (such as Fᵥ, F_{ab}, F_{ab′}, F_{(ab′)2} or other antigen-binding subsequences of Abs) that contain minimal sequence derived from non-human Ig.

Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody. Such “humanized” Abs are chimeric Abs, wherein substantially less than an intact human variable domain has been substituted by the corresponding sequence from a non-human species. In practice, humanized Abs are typically human Abs in which some CDR residues and possibly some FR residues are substituted by residues from analogous sites in rodent Abs. Humanized Abs include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fᵥ framework residues of the human Ig. Humanized Abs may comprise residues that are found neither in the recipient antibody nor in the imported CDR or framework sequences. In general, the humanized antibody comprises substantially all of at least one, and typically two, variable domains, in which most if not all of the CDR regions correspond to those of a nonhuman Ig and most if not all of the FR regions are those of a human Ig consensus sequence. The humanized antibody optimally also comprises at least a portion of an Ig constant region (F_{c}), typically that of a human Ig.

Human Abs can also be produced using various techniques, including phage display libraries and the preparation of human mAbs. Similarly, introducing human Ig genes into transgenic animals in which the endogenous Ig genes have been partially or completely inactivated can be exploited to synthesize human Abs. Upon challenge, human antibody production is observed, which closely resembles that seen in humans in all respects, including gene rearrangement, assembly, and antibody repertoire.

Bi-Specific mAbs

Bi-specific Abs are monoclonal, preferably human or humanized, that have binding specificities for at least two different antigens. For example, a binding specificity is NFAT5/TonEBP; the other is for any antigen of choice, preferably a cell-surface protein or receptor or receptor subunit. Traditionally, the recombinant production of bi-specific Abs is based on the co-expression of two Ig heavy-chain/light-chain pairs, where the two heavy chains have different specificities. Because of the random assortment of Ig heavy and light chains, the resulting hybridomas (quadromas) produce a potential mixture of ten different antibody molecules, of which only one has the desired bi-specific structure. The desired antibody can be purified using affinity chromatography or other techniques.

To manufacture a bi-specific antibody, variable domains with the desired antibody-antigen combining sites are fused to Ig constant domain sequences. The fusion is preferably with an Ig heavy-chain constant domain, comprising at least part of the hinge, CH2, and CH3 regions. Preferably, the first heavy-chain constant region (CH1) containing the site necessary for light-chain binding is in at least one of the fusions. DNAs encoding the Ig heavy-chain fusions and, if desired, the Ig light chain, are inserted into separate expression vectors and are co-transfected into a suitable host organism.

The interface between a pair of antibody molecules can be engineered b maximize the percentage of heterodimers that are recovered from recombinant cell culture. The preferred interface comprises at least part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This mechanism increases the yield of the heterodimer over unwanted end products such as homodimers.

Bi-specific Abs can be prepared as full length Abs or antibody fragments (e.g., F_{(ab′)2} bi-specific Abs). One technique to generate bi-specific Abs exploits chemical linkage. Intact Abs can be proteolytically cleaved to generate F_{(ab′)2} fragments. Fragments are reduced with a dithiol complexing agent, such as sodium arsenite, to stabilize vicinal dithiols and prevent intermolecular disulfide formation. The generated F_{ab′} fragments are then converted to thionitrobenzoate (TNB) derivatives. One of the F_{ab′}-TNB derivatives is then reconverted to the F_{ab′}-thiol by reduction with mercaptoethylamine and is mixed with an equimolar amount of the other F_{ab′}-TNB derivative to form the bi-specific antibody. The produced bi-specific Abs can be used as agents for the selective immobilization of enzymes.

F_{ab′} fragments may be directly recovered from E. coli and chemically coupled to form bi-specific Abs. For example, fully humanized bi-specific F_{(ab′)2} Abs can be produced by methods known to those of skill in the art. Each F_{ab′} fragment is separately secreted from E. coli and directly coupled chemically in vitro, forming the bi-specific antibody.

Various techniques for making and isolating bi-specific antibody fragments directly from recombinant cell culture have also been described. For example, leucine zipper motifs can be exploited. Peptides from the Fos and Jun proteins are linked to the F_{ab′} portions of two different Abs by gene fusion. The antibody homodimers are reduced at the hinge region to form monomers and then re-oxidized to form antibody heterodimers. This method can also produce antibody homodimers. The “diabody” technology provides an alternative method to generate bi-specific antibody fragments. The fragments comprise a heavy-chain variable domain (V_{H}) connected to a light-chain variable domain (V_{L}) by a linker that is too short to allow pairing between the two domains on the same chain. The V_{H} and V_{L} domains of one fragment are forced to pair with the complementary V_{L} and V_{H} domains of another fragment, forming two antigen-binding sites. Another strategy for making bi-specific antibody fragments is the use of single-chain Fᵥ(sFᵥ) dimers. Abs with more than two valences are also contemplated, such as tri-specific Abs.

Exemplary bi-specific Abs may bind to two different epitopes on a given NFAT5/TonEBP. Alternatively, cellular defense mechanisms can be restricted to a particular cell expressing the particular NFAT5/TonEBP: an anti-NFAT5/TonEBP arm may be combined with an arm that binds to a leukocyte triggering molecule, such as a T-cell receptor molecule (e.g., CD2, CD3, CD28, or B7), or to F_{c} receptors for IgG (F_{c}γR), such as F_{c}γRI (CD64), F_{c}γRII (CD32) and F_{c}γRIII (CD16). Bi-specific Abs may also be used to target cytotoxic agents to cells that express a particular NFAT5/TonEBP. These Abs possess a NFAT5/TonEBP-binding arm and an arm that binds a cytotoxic agent or a radionuclide chelator.

Heteroconjugate Abs

Heteroconjugate Abs, consisting of two covalently joined Abs, have been proposed to target immune system cells to unwanted cells and for treatment of human immunodeficiency virus (HIV) infection. Abs prepared in vitro using synthetic protein chemistry methods, including those involving cross-linking agents, are contemplated. For example, immunotoxins may be constructed using a disulfide exchange reaction or by forming a thioether bond. Examples of suitable reagents include iminothiolate and methyl-4-mercaptobutyrimidate.

Immunoconjugates

Immunoconjugates may comprise an antibody conjugated to a cytotoxic agent such as a chemotherapeutic agent, toxin (e.g., an enzymatically active toxin or fragment of bacterial, fungal, plant, or animal origin), or a radioactive isotope (i.e., a radioconjugate).

Useful enzymatically-active toxins and fragments include Diphtheria A chain, non-binding active fragments of Diphtheria toxin, exotoxin A chain from Pseudomonas aeruginosa, ricin A chain, abrin A chain, modeccin A chain, α-sarcin, Afeurites fordii proteins, Dianthin proteins, Phytolaca americana proteins, Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated Abs, such as ²¹²Bi, ¹³¹I, ¹³¹In, ⁹⁰Y, and ¹⁸⁶Re.

Conjugates of the antibody and cytotoxic agent are made using a variety of bi-functional protein-coupling agents, such as N-succinimidyl-3-(2-pyridyldithiol) propionate (SPDP), iminothiolane (IT), bifunctional derivatives of imidoesters (such as dimethyl adipimidate HCI), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared by methods known to those of skill in the art. ¹⁴C-labeled 1-isothiocyanatobenzyl-3-methyldiethylene triaminepentaacetic acid (MX-DTPA) is an exemplary chelating agent for conjugating radionuclide to antibody.

In another embodiment, the antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a streptavidin “ligand” (e.g., biotin) that is conjugated to a cytotoxic agent (e.g., a radionuclide).

Effector Function Engineering

The antibody can be modified to enhance its effectiveness in treating a disease, such as cancer or an autoimmune disease. For example, cysteine residue(s) may be introduced into the F_{c} region, thereby allowing interchain disulfide bond formation in this region. Such homodimeric Abs may have improved internalization capability and/or increased complement-mediated cell killing and antibody-dependent cellular cytotoxicity (ADCC). Homodimeric Abs with enhanced anti-tumor activity can be prepared using hetero-bifunctional cross-linkers by methods known to those of skill in the art. Alternatively, an antibody engineered with dual F_{c} regions may have enhanced complement lysis.

Diagnostic Applications of Abs Directed Against NFAT5/TonEBP

Anti-NFAT5/TonEBP Abs can be used to localize and/or quantitate NFAT5/TonEBP (e.g., for use in measuring levels of NFAT5/TonEBP within tissue samples or for use in diagnostic methods, etc.). Anti-NFAT5/TonEBP epitope Abs can be utilized as pharmacologically active compounds and screened according to the methods of the present invention.

Anti-NFAT5/TonEBP Abs can be used to isolate NFAT5/TonEBP by standard techniques, such as immunoaffinity chromatography or immunoprecipitation. These approaches facilitate purifying endogenous NFAT5/TonEBP antigen-containing polypeptides from cells and tissues. These approaches, as well as others, can be used to detect NFAT5/TonEBP in a sample to evaluate the abundance and pattern of expression of the antigenic protein Anti-NFAT5/TonEBP Abs can be used to monitor protein levels in tissues as part of a clinical testing procedure; for example, to determine the efficacy of a given treatment regimen. Coupling the antibody to a detectable substance (label) allows detection of Ab-antigen complexes. Classes of labels include fluorescent, luminescent, bioluminescent, and radioactive materials, enzymes and prosthetic groups. Useful labels include horseradish peroxidase, alkaline phosphatase, β-galactosidase, acetylcholinesterase, streptavidin/biotin, avidin/biotin, umbelliferone, fluorescein, fluorescein isothiocyanate, rhodamine, dichlorotriazinylamine fluorescein, dansyl chloride, phycoerythrin, luminol, luciferase, luciferin, aequorin, and ¹²⁵I, ¹³¹I, ³⁵S or ³H.

Antibody Therapeutics

Abs of the invention, including polyclonal, monoclonal, humanized and fully human Abs, can be used therapeutically. Such agents will generally be employed to treat or prevent a disease or pathology in a subject. An antibody preparation, preferably one having high antigen specificity and affinity generally mediates an effect by binding the target epitope(s). Generally, administration of such Abs may mediate one of two effects: (1) the antibody may prevent ligand binding, eliminating endogenous ligand binding and subsequent signal transduction, or (2) the antibody elicits a physiological result by binding an effector site on the target molecule, initiating signal transduction.

A therapeutically effective amount of an antibody relates generally to the amount needed to achieve a therapeutic objective, epitope binding affinity, administration rate, and depletion rate of the antibody from a subject. Common ranges for therapeutically effective doses may be, as a nonlimiting example, from about 0.1 mg/kg body weight to about 50 mg/kg body weight. Dosing frequencies may range, for example, from twice daily to once a week.

Pharmaceutical Compositions for Abs

Anti-NFAT5/TonEBP Abs, as well as other NFAT5/TonEBP interacting molecules (such as aptamers) identified in other assays, can be administered in pharmaceutical compositions as disclosed, infra, to treat various disorders. Abs that are internalized are preferred when whole Abs are used as inhibitors. Liposomes may also be used as a delivery vehicle for intracellular introduction. Where antibody fragments are used, the smallest inhibitory fragment that specifically binds to the epitope is preferred. For example, peptide molecules can be designed that bind a preferred epitope based on the variable-region sequences of a useful antibody. Such peptides can be synthesized chemically and/or produced by recombinant DNA technology. Formulations may also contain more than one active compound for a particular treatment, preferably those with activities that do not adversely affect each other. The composition may comprise an agent that enhances function, such as a cytotoxic agent, cytokine, chemotherapeutic agent, or growth-inhibitory agent.

The active ingredients can also be entrapped in microcapsules prepared by coacervation techniques or by interfacial polymerization; for example, hydroxymethylcellulose or gelatin-microcapsules and poly-(methylmethacrylate) microcapsules, respectively, in colloidal drug delivery systems (for example, liposomes, albumin microspheres, microemulsions, nanoparticles, and nanocapsules) or in macroemulsions. The formulations to be used for in vivo administration are highly preferred to be sterile. This is readily accomplished by filtration through sterile filtration membranes or any of a number of techniques.

Sustained-release preparations may also be prepared, such as semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as injectable microspheres composed of lactic acid-glycolic acid copolymer, and poly-D-(−)-3-hydroxybutyric acid. While polymers such as ethylene-vinyl acetate and lactic acid-glycolic acid enable release of molecules for over 100 days, certain hydrogels release proteins for shorter time periods and may be preferred.

In various aspects, the inhibitor can also be selected from the group consisting of a ribozyme, antisense compound, triplex-forming molecule, siRNA, and aptamer.

Ribozymes

Ribozyme molecules designed to catalytically cleave NFAT5/TonEBP mRNA transcripts can also be used to prevent translation of NFAT5/TonEBP mRNAs and expression of a NFAT5/TonEBP protein (see, e.g., Wright and Kearney, Cancer Invest. 19:495, 2001; Lewin and Hauswirth, Trends Mol. Med. 7:221, 2001; Sarver et al. (1990) Science 247:1222-1225 and U.S. Pat. No. 5,093,246). As one example, hammerhead ribozymes that cleave mRNAs at locations dictated by flanking regions that form complementary base pairs with the target mRNA might be used so long as the target mRNA has the following common sequence: 5′-UG-3′. See, e.g., Haseloff and Gerlach (1988) Nature 334:585-591. As another example, hairpin and hepatitis delta virus ribozymes may also be used. See, e.g., Bartolome et al. (2004) Minerva Med. 95(1):11-24. To increase efficiency and minimize the intracellular accumulation of non-functional mRNA transcripts, a ribozyme should be engineered so that the cleavage recognition site is located near the 5′ end of the target NFAT5/TonEBP mRNA. Ribozymes within the invention can be delivered to a cell using a vector as described herein.

Other methods can also be used to reduce NFAT5/TonEBP gene expression in a cell. For example, NFAT5/TonEBP gene expression can be reduced by inactivating or “knocking out” the NFAT5/TonEBP gene or its promoter using targeted homologous recombination. See, e.g., Kempin et al., Nature 389: 802 (1997); Smithies et al. (1985) Nature 317:230-234; Thomas and Capecchi (1987) Cell 51:503-512; and Thompson et al. (1989) Cell 5:313-321. For example, a mutant, non-functional NFAT5/TonEBP gene variant (or a completely unrelated DNA sequence) flanked by DNA homologous to the endogenous NFAT5/TonEBP gene (either the coding regions or regulatory regions of the NFAT5/TonEBP gene) can be used, with or without a selectable marker and/or a negative selectable marker, to transfect cells that express NFAT5/TonEBP protein in vivo.

NFAT5/TonEBP gene expression might also be reduced by targeting deoxyribonucleotide sequences complementary to the regulatory region of the NFAT5/TonEBP gene (i.e., the NFAT5/TonEBP promoter and/or enhancers) to form triple helical structures that prevent transcription of the NFAT5/TonEBP gene in target cells. See generally, Helene, C. (1991) Anticancer Drug Des. 6(6): 569-84; Helene, C., et al. (1992) Ann. N.Y. Acad. Sci. 660:27-36; and Maher, L. J. (1992) Bioassays 14(12): 807-15. Nucleic acid molecules to be used in this technique are preferably single stranded and composed of deoxyribonucleotides. The base composition of these oligonucleotides should be selected to promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, e.g., containing a stretch of G residues. These molecules will form a triple helix with a DNA duplex that is rich in GC pairs, in which the majority of the purine residues are located on a single strand of the targeted duplex, resulting in CGC triplets across the three strands in the triplex. The potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

The antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramide chemical synthesis. RNA molecules may be generated by in vitro and in vivo transcription of DNA sequences encoding the antisense RNA molecule. Such DNA sequences may be incorporated into a wide variety of vectors which incorporate suitable RNA polymerase promoters. Alternatively, antisense cDNA constructs that synthesize antisense RNA constitutively or inducibly, depending on the promoter used, can be introduced stably into cell lines.

Anti-Sense Nucleic Acids

Using antisense and sense NFAT5/TonEBP oligonucleotides can prevent NFAT5/TonEBP polypeptide expression. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are singe-stranded nucleic acids, either RNA or DNA, which can bind target NFAT5/TonEBP mRNA (sense) or NFAT5/TonEBP DNA (antisense) sequences. Anti-sense nucleic acids can be designed according to Watson and Crick or Hoogsteen base pairing rules. The anti-sense nucleic acid molecule can be complementary to the entire coding region of NFAT5/TonEBP mRNA, but more preferably, to only a portion of the coding or noncoding region of NFAT5/TonEBP mRNA. For example, the anti-sense oligonucleotide can be complementary to the region surrounding the translation start site of NFAT5/TonEBP mRNA. Antisense or sense oligonucleotides may comprise a fragment of the NFAT5/TonEBP DNA coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Methods to derive antisense or a sense oligonucleotides from a given cDNA sequence are known in the art.

Examples of modified nucleotides that can be used to generate the anti-sense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine. Alternatively, the anti-sense nucleic acid can be produced biologically using an expression vector into which a nucleic acid has been sub-cloned in an anti-sense orientation such that the transcribed RNA will be complementary to a target nucleic acid of interest.

To introduce antisense or sense oligonucleotides into target cells (cells containing the target nucleic acid sequence), any gene transfer method may be used. Examples of gene transfer methods include (1) biological, such as gene transfer vectors like Epstein-Barr virus or conjugating the exogenous DNA to a ligand-binding molecule, (2) physical, such as electroporation and injection, and (3) chemical, such as CaPO₄ precipitation and oligonucleotide-lipid complexes.

An antisense or sense oligonucleotide is inserted into a suitable gene transfer retroviral vector. A cell containing the target nucleic acid sequence is contacted with the recombinant retroviral vector, either in vivo or ex vivo. Examples of suitable retroviral vectors include those derived from the murine retrovirus M-MuLV, N2 (a retrovirus derived from M-MuLV), or the double copy vectors designated DCT5A, DCT5B and DCT5C. To achieve sufficient nucleic acid molecule transcription, vector constructs in which the transcription of the anti-sense nucleic acid molecule is controlled by a strong pol II or pol III promoter are preferred.

To specify target cells in a mixed population of cells cell surface receptors that are specific to the target cells can be exploited. Antisense and sense oligonucleotides can be conjugated to a ligand-binding molecule. Ligands are chosen for receptors that are specific to the target cells. Examples of suitable ligand-binding molecules include cell surface receptors, growth factors, cytokines, or other ligands that bind to cell surface receptors or molecules. Preferably, conjugation of the ligand-binding molecule does not substantially interfere with the ability of the receptors or molecule to bind the ligand-binding molecule conjugate, or block entry of the sense or antisense oligonucleotide or its conjugated version into the cell.

Liposomes efficiently transfer sense or an antisense oligonucleotide to cells. The sense or antisense oligonucleotide-lipid complex is preferably dissociated within the cell by an endogenous lipase.

The anti-sense nucleic acid molecule of the invention may be an α-anomeric nucleic acid molecule. An α-anomeric nucleic acid molecule forms specific double-stranded hybrids with complementary RNA in which, contrary to the usual α-units, the strands run parallel to each other. The ant-sense nucleic acid molecule can also comprise a 2′-o-methylribonucleotide or a chimeric RNA-DNA analogue.

Modifications of antisense and sense oligonucleotides can augment their effectiveness. Modified sugar-phosphodiester bonds or other sugar linkages, increase in vivo stability by conferring resistance to endogenous nucleases without disrupting binding specificity to target sequences. Other modifications can increase the affinities of the oligonucleotides for their targets, such as covalently linked organic moieties or poly-(L)-lysine. Other attachments modify binding specificities of the oligonucleotides for their targets, including metal complexes or intercalating (e.g., ellipticine) and alkylating agents.

For example, the deoxyribose phosphate backbone of the nucleic acids can be modified to generate peptide nucleic acids. “Peptide nucleic acids” or “PNAs” refer to nucleic acid mimics (e.g., DNA mimics) in that the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols known to those of skill in the art.

PNAs of NFAT5/TonEBP can be used in therapeutic and diagnostic applications. For example, PNAs can be used as anti-sense or antigene agents for sequence-specific modulation of gene expression by inducing transcription or translation arrest or inhibiting replication. NFAT5/TonEBP PNAs may also be used in the analysis of single base pair mutations (e.g., PNA directed PCR clamping; as artificial restriction enzymes when used in combination with other enzymes, e.g., S₁ nucleases, or as probes or primers for DNA sequence and hybridization.

PNAs of NFAT5/TonEBP can be modified to enhance their stability or cellular uptake. Lipophilic or other helper groups may be attached to PNAs, PNA-DNA dimmers formed, or the use of liposomes or other drug delivery techniques. For example, PNA-DNA chimeras can be generated that may combine the advantageous properties of PNA and DNA. Such chimeras allow DNA recognition enzymes (e.g., RNase H and DNA polymerases) to interact with the DNA portion while the PNA portion provides high binding affinity and specificity. PNA-DNA chimeras can be linked using linkers of appropriate lengths selected in terms of base stacking, number of bonds between the nucleobases, and orientation. The synthesis of PNA-DNA chimeras can be performed by methods known to those of skill in the art. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. PNA monomers are then coupled in a stepwise manner to produce a chimeric molecule with a 5′ PNA segment and a 3′ DNA segment Alternatively, chimeric molecules can be synthesized with a 5′ DNA segment and a 3′ PNA segment.

The oligonucleotide may include other appended groups such as peptides (e.g., for targeting host cell receptors in vivo), or agents facilitating transport across the cell membrane or the blood-brain barrier. In addition, oligonucleotides can be modified with hybridization-triggered cleavage agents or intercalating agents. The oligonucleotide may be conjugated to another molecule, e.g., a peptide, a hybridization triggered cross-linking agent, a transport agent, a hybridization-triggered cleavage agent and the like.

Triple-Helix Molecules

To inhibit transcription, triple-helix nucleic acids that are single-stranded and comprise deoxynucleotides are useful antagonists. These oligonucleotides are designed such that triple-helix formation via Hoogsteen base-pairing rules is promoted, generally requiring stretches of purines or pyrimidines.

Aptamers

Aptamers are short oligonucleotide sequences that can be used to recognize and specifically bind almost any molecule. The systematic evolution of ligands by exponential enrichment (SELEX) process (see, Ausubel, supra) is powerful and can be used to find such aptamers. Aptamers have many diagnostic and clinical uses; almost any use in which an antibody has been used clinically or diagnostically, aptamers too may be used. In addition, are cheaper to make once they have been identified, and can be easily applied in a variety of formats, including administration in pharmaceutical compositions, in bioassays, and diagnostic tests.

RNA Interference (RNAi)

The use of short-interfering RNA (siRNA) is a technique known in the art for inhibiting expression of a target gene by introducing exogenous RNA into a living cell (Elbashir et al. 2001. Nature. 411:494-498). siRNAs suppress gene expression through a highly regulated enzyme-mediated process called RNA interference (RNAi). RNAi involves multiple RNA-protein interactions characterized by four major steps: assembly of siRNA with the RNA-induced silencing complex (RISC), activation of the RISC, target recognition and target cleavage. Therefore, identifying siRNA-specific features likely to contribute to efficient processing at each step is beneficial efficient RNAi. Reynolds et al. provide methods for identifying such features. A. Reynolds et al., “Rational siRNA design for RNA interference”, Nature Biotechnology 22(3), March 2004. In that study, eight characteristics associated with siRNA functionality were identified: low G/C content, a bias towards low internal stability at the sense strand 3′-terminus, lack of inverted repeats, and sense strand base preferences (positions 3, 10, 13 and 19). Further analyses revealed that application of an algorithm incorporating all eight criteria significantly improves potent siRNA selection. siRNA sequences that contain internal repeats or palindromes may form internal fold-back structures. These hairpin-like structures may exist in equilibrium with the duplex form, reducing the effective concentration and silencing potential of the siRNA. The relative stability and propensity to form internal hairpins can be estimated by the predicted melting temperatures (T_{M}). Sequences with high Tm values would favor internal hairpin structures.

siRNA can be used either ex vivo or in vivo, making it useful in both research and therapeutic settings. Unlike in other antisense technologies, the RNA used in the siRNA technique has a region with double-stranded structure that is made identical to a portion of the target gene, thus making inhibition sequence-specific. Double-stranded RNA-mediated inhibition has advantages both in the stability of the material to be delivered and the concentration required for effective inhibition.

The extent to which there is loss of function of the target gene can be titrated using the dose of double stranded RNA delivered. A reduction or loss of gene expression in at least 99% of targeted cells has been shown. See, e.g., U.S. Pat. No. 6,506,559. Lower doses of injected material and longer times after administration of siRNA may result in inhibition in a smaller fraction of cells. Quantitation of gene expression in a cell show similar amounts of inhibition at the level of accumulation of target mRNA or translation of target protein.

The RNA used in this technique can comprise one or more strands of polymerized ribonucleotides, and modification can be made to the sugar-phosphate backbone as disclosed above. The double-stranded structure is often formed using either a single self-complementary RNA strand (hairpin) or two complementary RNA strands. RNA containing a nucleotide sequences identical to a portion of the target gene is preferred for inhibition, although sequences with insertions, deletions, and single point mutations relative to the target sequence can also be used for inhibition. Sequence identity may be optimized using alignment algorithms known in the art and through calculating the percent difference between the nucleotide sequences. The duplex region of the RNA could also be described in functional terms as a nucleotide sequence that is capable of hybridizing with a portion of the target gene transcript.

siRNA can often be a more effective therapeutic tool than other types of gene suppression due to siRNA's potent gene inhibition and ability to target receptors with a specificity can reach down to the level of single-nucleotide polymorphisms. Such specificity generally results in fewer side effects than is seen in conventional therapies, because other genes are not be affected by application of a sufficiently sequence-specific siRNA.

There are multiple ways to deliver siRNA to the appropriate target. Standard transfection techniques may be used, in which siRNA duplexes are incubated with cells of interest and then processed using standard commercially available kits. Electroporation techniques of transfection may also be appropriate. Cells or organisms can be soaked in a solution of the siRNA, allowing the natural uptake processes of the cells or organism to introduce the siRNA into the system. Viral constructs packaged into a viral particle would both introduce the siRNA into the cell line or organism and also initiate transcription through the expression construct. Other methods known in the art for introducing nucleic acids to cells may also be used, including lipid-mediated carrier transport, chemical-mediated transport, such as calcium phosphate, and the like.

For therapeutic uses, tissue-targeted nanoparticles may serve as a delivery vehicle for siRNA These nanoparticles carry the siRNA exposed on the surface, which is then available to bind to the target gene to be silenced. Schiffelers, et al., Nucleic Acids Research 2004 32(19):e149. These nanoparticles may be introduced into the cells or organisms using the above described techniques already known in the art. RGD peptides have been shown to be effective at targeting the neovasculature that accompanies the growth of tumors. Designing the appropriate nanoparticles for a particular illness is a matter of determining the appropriate targets for the particular disease. In the case of diabetes and pancreatic cancer, the present invention has already revealed potential targets for this powerful therapy.

Other delivery vehicles for therapeutic uses in humans include pharmaceutical compositions, intracellular injection, and intravenous introduction into the vascular system. Inhibition of gene expression can be confirmed by using biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression may be assayed using a reporter or drug resistance gene whose protein product can be easily detected and quantified. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin. These techniques are well known and easily practiced by those skilled in the art. For in vivo use in humans, reduction or elimination of symptoms of illness will confirm inhibition of the target gene's expression.

In yet another aspect of the present invention, a method is provided for diagnosing a cancer or an autoimmune disease in a subject comprising (a) obtaining a sample from the subject; (b) detecting NFAT5/TonEBP expression in the sample; and (c) comparing to the expression of NFAT5/TonEBP of the sample to a control sample, wherein an elevated expression of NFAT5/TonEBP in the sample is diagnostic for cancer. In various aspects, (b) can comprise detecting NFAT5/TonEBP mRNA. Alternatively, (b) can comprise detection of NFAT5/TonEBP protein.

Detection Assays

Portions or fragments of NFAT5/TonEBP cDNA sequences identified herein (and the complete NFAT5/TonEBP gene sequences) are useful in themselves. By way of non-limiting example, these sequences can be used to: (1) identify an individual from a minute biological sample (tissue typing); and (2) aid in forensic identification of a biological sample.

The NFAT5/TonEBP sequences of the invention can be used to identify individuals from minute biological samples. In this technique, an individual's genomic DNA is digested with one or more restriction enzymes and probed on a Southern blot to yield unique bands. The sequences of the invention are useful as additional DNA markers for “restriction fragment length polymorphisms” (RFLP).

Furthermore, the NFAT5/TonEBP sequences can be used to determine the actual base-by-base DNA sequence of targeted portions of an individual's genome. NFAT5/TonEBP sequences can be used to prepare two PCR primers from the 5′- and 3′-termini of the sequences that can then be used to amplify an the corresponding sequences from an individual's genome and then sequence the amplified fragment.

Panels of corresponding DNA sequences from individuals can provide unique individual identifications, as each individual will have a unique set of such DNA sequences due to allelic differences. The sequences of the invention can be used to obtain such identification sequences from individuals and from tissue. The NFAT5/TonEBP sequences of the invention uniquely represent portions of an individual's genome. Allelic variation occurs to some degree in the coding regions of these sequences, and to a greater degree in the noncoding regions. The allelic variation between individual humans occurs with a frequency of about once ever 500 bases. Much of the allelic variation is due to single nucleotide polymorphisms (SNPs), which include RFLPs.

Each of the sequences described herein can, to some degree, be used as a standard against which DNA from an individual can be compared for identification purposes. Because greater numbers of polymorphisms occur in noncoding regions, fewer sequences are necessary to differentiate individuals. Noncoding sequences can positively identify individuals with a panel of 10 to 1,000 primers that each yield a noncoding amplified sequence of 100 bases. If predicted coding sequences, such as those in SEQ ID NOs: 2, 4, 6, 8 and 10 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to treat an individual prophylactically. Accordingly, one aspect of the invention relates to diagnostic assays for determining NFAT5/TonEBP and/or nucleic acid expression as well as NFAT5/TonEBP activity, in the context of a biological sample (e.g., blood, serum, cells, tissue) to determine whether an individual is afflicted with a disease or disorder, or is at risk of developing a disorder, associated with aberrant NFAT5/TonEBP expression or activity, including cancer. The invention also provides for prognostic (or predictive) assays for determining whether an individual is at risk of developing a disorder associated with NFAT5/TonEBP, nucleic acid expression or activity. For example, mutations in NFAT5/TonEBP can be assayed in a biological sample. Such assays can be used for prognostic or predictive purpose to prophylactically treat an individual prior to the onset of a disorder characterized by or associated with NFAT5/TonEBP, nucleic acid expression, or biological activity.

Another aspect of the invention provides methods for determining NFAT5/TonEBP activity, or nucleic acid expression, in an individual to select appropriate therapeutic or prophylactic agents for that individual (referred to herein as “pharmacogenomics”). Pharmacogenomics allows for the selection of modalities (e.g., drugs, foods) for therapeutic or prophylactic treatment of an individual based on the individual's genotype (e.g., the individual's genotype to determine the individual's ability to respond to a particular agent). Another aspect of the invention pertains to monitoring the influence of modalities (e.g., drugs, foods) on the expression or activity of NFAT5/TonEBP in clinical trials.

Diagnostic Assays

An exemplary method for detecting the presence or absence of NFAT5/TonEBP in a biological sample involves obtaining a biological sample from a subject and contacting the biological sample with a compound or an agent capable of detecting NFAT5/TonEBP or NFAT5/TonEBP nucleic acid (e.g., mRNA, genomic DNA) such that the presence of NFAT5/TonEBP is confirmed in the sample. An agent for detecting NFAT5/TonEBP mRNA or genomic DNA is a labeled nucleic acid probe that can hybridize to NFAT5/TonEBP mRNA or genomic DNA. The nucleic acid probe can be, for example, a full-length NFAT5/TonEBP nucleic acid, such as the nucleic acids of SEQ ID NOs: 2, 4, 6, 8 and 10, or portions thereof, such as an oligonucleotide of at least 15, 30, 50, 100, 250 or 500 nucleotides in length and sufficient to specifically hybridize under stringent conditions to NFAT5/TonEBP mRNA or genomic DNA.

An agent for detecting NFAT5/TonEBP polypeptide is an antibody capable of binding to NFAT5/TonEBP, preferably an antibody with a detectable label. Abs can be polyclonal, or more preferably, monoclonal. An intact antibody, or a fragment (e.g., F_{ab} or F(ab′)₂) can be used. A labeled probe or antibody is coupled (i.e., physically linking) to a detectable substance, as well as indirect detection of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently-labeled streptavidin. The term “biological sample” includes tissues, cells and biological fluids isolated from a subject, as well as tissues, cells and fluids present within a subject. The detection method of the invention can be used to detect NFAT5/TonEBP mRNA, protein, or genomic DNA in a biological sample in vitro as well as in vivo. For example, in vitro techniques for detection of NFAT5/TonEBP mRNA include Northern hybridizations and in situ hybridizations. In vitro techniques for detection of NFAT5/TonEBP polypeptide include enzyme linked immunosorbent assays (ELISAs), Western blots, immunoprecipitations, and immunofluorescence. In vitro techniques for detection of NFAT5/TonEBP genomic DNA include Southern hybridizations and fluorescence in situ hybridization (FISH). Furthermore, in vivo techniques for detecting NFAT5/TonEBP include introducing into a subject a labeled anti-NFAT5/TonEBP antibody. For example, the antibody can be labeled with a radioactive marker whose presence and location in a subject can be detected by standard imaging techniques.

In one embodiment, the biological sample from the subject contains protein molecules, and/or mRNA molecules, and/or genomic DNA molecules. A preferred biological sample is blood. In another embodiment, the methods further involve obtaining a biological sample from a subject to provide a control, contacting the sample with a compound or agent to detect NFAT5/TonEBP, mRNA, or genomic DNA, and comparing the presence of NFAT5/TonEBP, mRNA or genomic DNA in the control sample with the presence of NFAT5/TonEBP, mRNA or genomic DNA in the test sample.

The invention also encompasses kits for detecting NFAT5/TonEBP in a biological sample. For example, the kit can comprise: a labeled compound or agent capable of detecting NFAT5/TonEBP or NFAT5/TonEBP mRNA in a sample; reagent and/or equipment for determining the amount of NFAT5/TonEBP in the sample; and reagent and/or equipment for comparing the amount of NFAT5/TonEBP in the sample with a standard. The compound or agent can be packaged in a suitable container. The kit can further comprise instructions for using the kit to detect NFAT5/TonEBP or nucleic acid.

Prognostic Assays

The diagnostic methods described herein can furthermore be utilized to identify subjects having or at risk of developing a disease or disorder associated with aberrant NFAT5/TonEBP expression or activity. For example, the assays described herein, can be used to identify a subject having or at risk of developing a disorder associated with NFAT5/TonEBP, nucleic acid expression or activity. Alternatively, the prognostic assays can be used to identify a subject having or at risk for developing a disease or disorder. The invention provides a method for identifying a disease or disorder associated with aberrant NFAT5/TonEBP expression or activity in which a test sample is obtained from a subject and NFAT5/TonEBP or nucleic acid (e.g., mRNA, genomic DNA) is detected. A test sample is a biological sample obtained from a subject. For example, a test sample can be a biological fluid (e.g., serum), cell sample, or tissue.

Prognostic assays can be used to determine whether a subject can be administered a modality (e.g., an agonist, antagonist, peptidomimetic, protein, peptide, nucleic acid, small molecule, food, etc.) to treat a disease or disorder associated with aberrant NFAT5/TonEBP expression or activity. Such methods can be used to determine whether a subject can be effectively treated with an agent for a disorder. The invention provides methods for determining whether a subject can be effectively treated with an agent for a disorder associated with aberrant NFAT5/TonEBP expression or activity in which a test sample is obtained and NFAT5/TonEBP or nucleic acid is detected (e.g., where the presence of NFAT5/TonEBP or nucleic acid is diagnostic for a subject that can be administered the agent to treat a disorder associated with aberrant NFAT5/TonEBP expression or activity).

The methods of the invention can also be used to detect genetic lesions in a NFAT5/TonEBP to determine if a subject with the genetic lesion is at risk for a disorder. Methods include detecting, in a sample from the subject, the presence or absence of a genetic lesion characterized by at an alteration affecting the integrity of a gene encoding a NFAT5/TonEBP polypeptide, or the misexpression of NFAT5/TonEBP. Such genetic lesions can be detected by ascertaining: (1) a deletion of one or more nucleotides from NFAT5/TonEBP; (2) an addition of one or more nucleotides to NFAT5/TonEBP; (3) a substitution of one or more nucleotides in NFAT5/TonEBP, (4) a chromosomal rearrangement of a NFAT5/TonEBP gene; (5) an alteration in the level of a NFAT5/TonEBP mRNA transcripts, (6) aberrant modification of a NFAT5/TonEBP, such as a change genomic DNA methylation, (7) the presence of a non-wild-type splicing pattern of a NFAT5/TonEBP mRNA transcript, (8) a non-wild-type level of NFAT5/TonEBP, (9) allelic loss of NFAT5/TonEBP, and/or (10) inappropriate post-translational modification of NFAT5/TonEBP polypeptide. There are a large number of known assay techniques that can be used to detect lesions in NFAT5/TonEBP. Any biological sample containing nucleated cells may be used.

In certain embodiments, lesion detection may use a probe/primer in a polymerase chain reaction (PCR) (such as anchor PCR or rapid amplification of cDNA ends (RACE) PCR, or, alternatively, in a ligation chain reaction (LCR). This method may include collecting a sample from a patient, isolating nucleic acids from the sample, contacting the nucleic acids with one or more primers that specifically hybridize to NFAT5/TonEBP under conditions such that hybridization and amplification of the NFAT5/TonEBP (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication, transcriptional amplification system; Qβ Replicase, or any other nucleic acid amplification method, followed by the detection of the amplified molecules using techniques well known to those of skill in the art. These detection schemes are especially useful for the detection of nucleic acid molecules present in low abundance and are known to those of skill in the art.

Mutations in NFAT5/TonEBP from a sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes can be used to score for the presence of specific mutations by development or loss of a ribozyme cleavage site.

Hybridizing a sample and control nucleic acids, e.g., DNA or RNA, to high-density arrays containing hundreds or thousands of oligonucleotides probes, can identify genetic mutations in NFAT5/TonEBP. For example, genetic mutations in NFAT5/TonEBP can be identified in two-dimensional arrays containing light-generated DNA probes. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NFAT5/TonEBP and detect mutations by comparing the sequence of the sample NFAT5/TonEBP-with the corresponding wild-type (control) sequence. Any of a variety of automated sequencing procedures can be used when performing diagnostic assays including sequencing by mass spectrometry.

Other methods for detecting mutations in the NFAT5/TonEBP include those in which protection from cleavage agents is used to detect mismatched bases in RNA/RNA or RNA/DNA heteroduplexes. In general, the technique of “mismatch cleavage” starts by providing heteroduplexes formed by hybridizing (labeled) RNA or DNA containing the wild-type NFAT5/TonEBP sequence with potentially mutant RNA or DNA obtained from a sample. The double-stranded duplexes are treated with an agent that cleaves single-stranded regions of the duplex such as those that arise from base pair mismatches between the control and sample strands. For instance, RNA/DNA duplexes can be treated with RNase and DNA/DNA hybrids treated with S₁ nuclease to enzymatically digest the mismatched regions. In other embodiments, either DNA/DNA or RNA/DNA duplexes can be treated with hydroxylamine or osmium tetroxide and with piperidine in order to digest mismatched regions. The digested material is then separated by size on denaturing polyacrylamide gels to determine the mutation site. The control DNA or RNA can be labeled for detection.

Mismatch cleavage reactions may employ one or more proteins that recognize mismatched base pairs in double-stranded DNA (DNA mismatch repair) in defined systems for detecting and mapping point mutations in NFAT5/TonEBP cDNAs obtained from samples of cells. For example, the mutY enzyme of E. coli cleaves A at G/A mismatches and the thymidine DNA glycosylase from HeLa cells cleaves T at G/T mismatches. According to an exemplary embodiment, a probe based on a wild-type NFAT5/TonEBP sequence is hybridized to a cDNA or other DNA product from a test cell(s). The duplex is treated with a DNA mismatch repair enzyme, and the cleavage products, if any, can be detected from electrophoresis protocols or the like.

Electrophoretic mobility alterations can be used to identify mutations in NFAT5/TonEBP. For example, single strand conformation polymorphism (SSCP) may be used to detect differences in electrophoretic mobility between mutant and wild type nucleic acids. Single-stranded DNA fragments of sample and control NFAT5/TonEBP nucleic acids are denatured and then renatured. The secondary structure of single-stranded nucleic acids varies according to sequence; the resulting alteration in electrophoretic mobility allows detection of even a single base change. The DNA fragments may be labeled or detected with labeled probes. The sensitivity of the assay may be enhanced by using RNA (rather than DNA), in which the secondary structure is more sensitive to a sequence changes. The subject method may use heteroduplex analysis to separate double stranded heteroduplex molecules on the basis of changes in electrophoretic mobility.

The migration of mutant or wild-type fragments can be assayed using denaturing gradient gel electrophoresis (DGGE). In DGGE, DNA is modified to prevent complete denaturation, for example by adding a GC clamp of approximately 40 bp of high-melting GC-rich DNA by PCR. A temperature gradient may also be used in place of a denaturing gradient to identify differences in the mobility of control and sample DNA.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. Such allele-specific oligonucleotides are hybridized to PCR-amplified target DNA or a number of different mutations when the oligonucleotides are attached to the hybridizing membrane and hybridized with labeled target DNA.

Alternatively, allele specific amplification technology that depends on selective PCR amplification may be used. Oligonucleotide primers for specific amplifications may carry the mutation of interest in the center of the molecule (so that amplification depends on differential hybridization) or at the extreme 3′-terminus of one primer where, under appropriate conditions, mismatch can prevent, or reduce polymerase extension. Novel restriction site in the region of the mutation may be introduced to create cleavage-based detection. Certain amplification may also be performed using Taq ligase for amplification. In such cases, ligation occurs only if there is a perfect match at the 3′-terminus of the 5′ sequence, allowing detection of a known mutation by scoring for amplification.

The described methods may be performed, for example, by using pre-packaged kits comprising at least one probe (nucleic acid or antibody) that may be conveniently used, for example, in clinical settings to diagnose patients exhibiting symptoms or family history of a disease or illness involving NFAT5/TonEBP.

Furthermore, any cell type or tissue in which NFAT5/TonEBP is expressed may be utilized in the prognostic assays described herein.

Pharmacogenomics

Agents, or modulators that have a stimulatory or inhibitory effect on NFAT5/TonEBP activity or expression, as identified by a screening assay can be administered to individuals to treat, prophylactically or therapeutically, disorders. In conjunction with such treatment, the pharmacogenomics (i.e., the study of the relationship between a subject's genotype and the subject's response to a foreign modality, such as a food, compound or drug) may be considered. Metabolic differences of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Pharmacogenomics can further be used to determine appropriate dosages and therapeutic regimens. Accordingly, the activity of NFAT5/TonEBP, expression of NFAT5/TonEBP nucleic acid, or NFAT5/TonEBP mutation(s) in an individual can be determined to guide the selection of appropriate agent(s) for therapeutic or prophylactic treatment.

Pharmacogenomics deals with clinically significant hereditary variations in the response to modalities due to altered modality disposition and abnormal action in affected persons. In general, two pharmacogenetic conditions can be differentiated: (1) genetic conditions transmitted as a single factor altering the interaction of a modality with the body (altered drug action) or (2) genetic conditions transmitted as single factors altering the way the body acts on a modality (altered drug metabolism). These pharmacogenetic conditions can occur either as rare defects or as nucleic acid polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) explains the phenomena of some patients who show exaggerated drug response and/or serious toxicity after taking the standard and safe dose of a drug. These polymorphisms are expressed in two phenotypes in the population, the extensive metabolizer (EM) and poor metabolizer (PM). The prevalence of PM is different among different populations. For example, the CYP2D6 gene is highly polymorphic and several mutations have been identified in PM, which all lead to the absence of functional CYP2D6. Poor metabolizers due to mutant CYP2D6 and CYP2C19 frequently experience exaggerated drug responses and side effects when they receive standard doses. If a metabolite is the active therapeutic moiety, PM shows no therapeutic response, as demonstrated for the analgesic effect of codeine mediated by its CYP2D6-formed metabolite morphine. At the other extreme are the so-called ultra-rapid metabolizers who are unresponsive to standard doses. Recently, the molecular basis of ultra-rapid metabolism has been identified to be due to CYP2D6 gene amplification.

The activity of NFAT5/TonEBP, expression of NFAT5/TonEBP nucleic acid, or mutation content of NFAT5/TonEBP in an individual can be determined to select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NFAT5/TonEBP modulator, such as a modulator identified by one of the described exemplary screening assays.

In another aspect of the present invention, a method for treating a cancer or an autoimmune disease is provided comprising decreasing the activity of NFAT5/TonEBP. In various aspects, decreasing the activity can comprise decreasing the expression of NFAT5/TonEBP. In various aspects, decreasing the expression comprises transforming a cell to express a polynucleotide anti-sense to at least a portion of an endogenous polynucleotide encoding NFAT5/TonEBP as described above. In various aspects, decreasing the activity comprises inhibiting, preventing, or reversing at least one activity of NFAT5/TonEBP as described above. In various other aspects, decreasing the activity can comprise transforming a cell to express an aptamer to NFAT5/TonEBP as described above. In various aspects, decreasing the activity can comprise introducing into a cell an aptamer to NFAT5/TonEBP as described above. In a further aspect, decreasing the activity can comprise administering to a cell an antibody that selectively binds NFAT5/TonEBP as described above.

Methods of Treatment

The invention provides for both prophylactic and therapeutic methods of treating a subject at risk of (or susceptible to) a disorder or having a disorder associated with aberrant NFAT5/TonEBP expression or activity. Examples include disorders in which cell metabolic demands (and consequently, demands on mitochondria and endoplasmic reticulum) are high, such as during rapid cell growth. Examples of such disorders and diseases include cancers, such as melanoma, breast cancer or colon cancer; autoimmune diseases, such as diabetes; and those further described, infra.

Treatment of Diseases and Disorders

Diseases and disorders that are characterized by increased NFAT5/TonEBP levels or biological activity (such as cancer and autoimmune disease) may be treated with therapeutics that antagonize (i.e., reduce or inhibit) activity. Antagonists may be administered in a therapeutic or prophylactic manner. Therapeutics that may be used include: (1) NFAT5/TonEBP peptides, or analogs, derivatives, fragments or homologues thereof; (2) Abs to a NFAT5/TonEBP peptide; (3) NFAT5/TonEBP nucleic acids; (4) administration of antisense nucleic acid and nucleic acids that are “dysfunctional” (i.e., due to a heterologous insertion within the coding sequences) that are used to eliminate endogenous function of by homologous recombination; or (5) modulators (i.e., inhibitors, agonists and antagonists, including additional peptide mimetic of the invention or Abs specific to NFAT5/TonEBP) that alter the interaction between NFAT5/TonEBP and its binding partner.

Diseases and disorders that are characterized by decreased NFAT5/TonEBP levels or biological activity may be treated with therapeutics that increase (i.e., are agonists to) activity. Therapeutics that upregulate activity may be administered therapeutically or prophylactically. Therapeutics that may be used include peptides, or analogs, derivatives, fragments or homologues thereof; or an agonist that increases bioavailability.

Increased or decreased levels can be readily detected by quantifying peptide and/or RNA, by obtaining a patient tissue sample (e.g., from biopsy tissue) and assaying in vitro for RNA or peptide levels, structure and/or activity of the expressed peptides (or NFAT5/TonEBP mRNAs). Methods include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Cancer

The primary application of this invention would be to utilize the osmotic stress response pathway as a means to screen for drugs that either inhibit or augment this pathway. Inhibitory drugs would be useful to block the pathway, thus sensitizing cells to hypertonic stress such that exposure to hypertonic stress would result in inhibition of cell growth and cell death. An example in which such a drug would be useful would be in the treatment of cancers. This invention demonstrates that cancer cells in vivo are exposed to hypertonic stress and significantly overexpress NFAT5/TonEBP. Inhibition of the osmotic stress response pathway would thus induce growth inhibition and cell death specifically of cancer cells, with minimal or no effects on normal cells, which express little or no NFAT5/TonEBP. The cancer etiology is further discussed in the Examples and described in the Figures.

In various aspects, the cancer can be selected from the group consisting of epithelial malignancies, sarcomas, and lymphomas. Other cancers are treatable using the inhibitors of the present invention as can be determined by those of skill in the art.

Inflammation

The expression of NFAT5/TonEBP RNA has been identified in proliferating synovial fibroblasts derived from the synovium of patients with rheumatoid arthritis via a subtractive hybridization approach, and went on to demonstrate expression by in situ hybridization using tissue sections. NFAT5/TonEB RNA was identified not only in fibroblast-like cells but also osteoclasts. Moreover, NFAT5 was preferentially expressed in the synovium of patients with rheumatoid arthritis and was not detectable in the synovium of normal individuals.

Although no suggestion was made as to the mechanism underlying the preferential expression of NFAT5/TonEBP in the diseased synovium, these results are consistent with the hypothesis proposed here that sites subject to an active acute inflammatory response are likely exposed to significant hyperosmotic stress. In the same manner that hypoxic stress is of relevance to not only cancer but to inflammatory processes as well, it would thus seem reasonable that osmotic stress also plays a role in inflammation.

Given the observations provided in this invention that cell death and degradation results in significant increase in the osmolality of the local environment, it is highly likely that the local environment at the site of inflammation is hyperosmotic. This is because a feature function of an acute inflammatory response is the elaboration of hydrolytic enzymes, as mediated by neutrophils in the site of acute inflammation. Therefore, one means of inhibiting such an inflammatory response would be to inhibit the osmotic stress response pathway. As a result, those cells most closely associated with the site of acute inflammation, which are in fact those cells that mediate the acute inflammatory process, would be rendered sensitive to hypertonicity induced cell death.

In various aspects, the autoimmune disease can be selected from the group consisting of rheumatoid arthritis, systemic lupus erythematosis, multiple sclerosis, and type I diabetes. Other autoimmune diseases are treatable using the inhibitors of the present invention as can be determined by those of skill in the art.

Other Diseases or Disorders

A drug (or non-drug) intervention that would enhance the osmotic stress pathway would be useful to enhance the survival of cells otherwise exposed to a hypertonic environment. An example in which such a drug would be useful would be in diseases associated with cell death, such as acute cerebrovascular disease (stroke) or acute myocardial infarction (heart attack) where the cell death resulting from the infarct increases local tissue osmolality. Such an increase in local osmolality would have a detrimental effect on adjacent non-infarcted but is chemically injured cells. A drug or intervention that would enhance the ability of these cells to survive within this hypertonic environment would result in a reduction in the overall area tissue damage associated with the initiating event.

In yet another aspect of the present invention, a method is provided for determining whether a compound up-regulates or down-regulates the transcription of a NFAT5/TonEBP gene, comprising contacting the compound with a RNA polymerase and said gene, followed by measuring NFAT5/TonEBP gene transcription initiated by the RNA polymerase acting on the gene, wherein measuring enhanced transcription is indicative of up-regulation and measuring decreased transcription is indicative of down-regulation. In various aspects, the contacting can occur in a cell. In another aspect, the compound can be selected from the group consisting of a ribozyme, antisense compound, triplex-forming molecule, siRNA, and aptamer as described above.

In another aspect of the present invention, a method is provided for determining whether a compound up-regulates or down-regulates translation of an NFAT5/TonEBP gene in a cell, comprising contacting the compound with the cell, the cell further comprising the NFAT5/TonEBP gene, and measuring NFAT5/TonEBP gene translation, wherein measuring enhanced translation is indicative of up-regulation and measuring decreased translation is indicative of down-regulation. In various aspects, the compound can be selected from the group consisting of a ribozyme, antisense compound, triplex-forming molecule, siRNA, and aptamer as described above.

In a further aspect of the present invention, a method is provided for determining whether a compound is a NFAT5/TonEBP target gene inhibitor, comprising: (a) culturing a first cell and a second cell under conditions of hypertonic stress; (b) contacting the first cell with a test agent; and (c) assaying the first cell and the second cell for at least one activity of a NFAT5/TonEBP target gene, wherein a decrease in activity of a NFAT5/TonEBP target gene in the first cell relative to the second cell indicates that the test agent is an NFAT5/TonEBP target gene inhibitor. In various aspects, the contacting can occur in a cell. In another aspect, the compound can be selected from the group consisting of a ribozyme, antisense compound, triplex-forming molecule, siRNA, and aptamer as described above.

In various aspects, the NFAT5/TonEBP target gene can be selected from the group consisting of aldose reductase (AR) which catalyzes reduction of glucose to sorbitol; sodium/myo-inositol cotransporter (SMIT) which transports myo-inositol across plasma membrane using the Na+ Clelectrochemical gradient; sodium/chloride/betaine cotransporter (BGT1) which transports betaine across plasma membrane using the Na+ Clelectrochemical gradient; urea transporter (UT-A) a vasopressin-regulated urea transporter; expressed primarily in renal medulla; taurine transporter (TauT) a membrane transporter for the amino acid taurine; heat shock protein 70 gene (HSP70-2) a molecular chaperone; protects from urea induced apoptosis; sodium-coupled neutral amino acid transporter-2 (ATA2, SNAT2) a system A neutral amino acid transporter; osmotic stress protein of 94 kDa (Osp94) a putative molecular chaperone based on sequence homology to heat shock proteins; and aquaporin 2 (AQP2) a plasma membrane water channel. The following Table 4 provides relevant information:

TABLE 4
NFAT5/TonEBP Target Genes

| Target gene¹ | Function | References |
|---|---|---|
| aidose reductase (AR) | catalyzes reduction at glucose to sorbitol | (Ko et al., 1997; Lopez-Rodriguez |
| | | et al., 2004) |
| sodium/myo-inositol cotransporter (SMIT) | transports myo-inositol across plasma | (Lopez-Rodriguez et al., 2004; Rim |
| | membrane using the Na+ Cl− | et al., 1998) |
| | electrochemical gradient | |
| sodium/chloride/betaine cotransporter (BGT 1) | transports betaine across plasma | (Lopez-Rodriguez et al., 2004, |
| | membrane using the Na+ Cl- | Miyakawa et al., 1999a) |
| | electrochemical gradient | |
| urea transporter (UT-A) | vasopressin-regulated urea transporter: | (Nakayama et al., 2000) |
| | expressed primarily in renal medulla | |
| taurine transporter (TauT)² | membrane transporter for the amino acid | (Ito et al., 2004) |
| | taurine | |
| heat shock protein 70 gene (HSP70-2) | molecular chaperone; protects from urea- | (Go et al., 2004; Woo et al., 2002) |
| | induced apoptosis | |
| sodium-coupled neutral amino acid | system A neutral amino acid transporter | (Trama et al., 2002) |
| transporter-2 (ATA2, SNAT2)³ | | |
| osmotic stress protein of 94 kDa (Osp94) | putative molecular chaperone based on | (Kojima et al., 2004) |
| | sequence homology to heat shock proteins | |
| aquaporin 2 (AQP2) | plasma membrane water channel | (Kasono et al., 2005, Lopez- |
| | | Rodriguez et al., 2004) |

¹Unless otherwise noted, identification of a gene as an NFAT5/TonEBP target is based on a combination or genetic and cell-based functional assays as well as direct DNA binding studies
²Although ex vivo assays indicate that NFAT5/TonEBP directly regulates the TauT gene (Ito et al., 2004), NFAT5 null mice exhibit no reduction in TauT expression in the kidney in contrast to the significantly reduced expression of AR, SMIT and BGT1 (Lopez-Rodriguez et al., 2004).
³Loss of NFAT5 function in vivo results in the impaired Induction of ATA2 mRNA In response to hypertonicity, thus providing functional genetic evidence that NFAT5 regulates ATA2 gene expression. However, direct regulation at the ATA2 gene by NFAT5 has not as yet been demonstrated. The ATA2 gene is thus most accurately considered a potential NFAT5-target gene

In yet another aspect of the present invention, a non-human transgenic animal is provided, wherein at least one NFAT5/TonEBP gene comprised by the non-human transgenic animal is disrupted. In various aspects, the NFAT5/TonEBP gene is not fully expressed. In another aspect, the non-human animal can be selected from the group consisting of a mouse, rat, dog, cat, cow, pig, horse, rabbit, frog, chicken, and sheep. In one aspect, the non-human transgenic animal is a mouse. Transgenic NFAT5/TonEBP animals,

“Transgenic animals” are non-human animals, preferably mammals, more preferably a rodents such as rats or mice, in which one or more of the cells include a transgene. Other transgenic animals include primates, sheep, dogs, cows, goats, chickens, amphibians, etc. A “transgene” is exogenous DNA that is integrated into the genome of a cell from which a transgenic animal develops, and that remains in the genome of the mature animal. Transgenes preferably direct the expression of an encoded gene product in one or more cell types or tissues of the transgenic animal with the purpose of preventing expression of a naturally encoded gene product in one or more cell types or tissues (a “knockout” transgenic animal; see the Examples), or serving as a marker or indicator of an integration, chromosomal location, or region of recombination (e.g., cre/IoxP mice). A “homologous recombinant animal” is a non-human animal, such as a rodent, in which endogenous NFAT5/TonEBP has been altered by an exogenous DNA molecule that recombines homologously with endogenous NFAT5/TonEBP in a (e.g., embryonic) cell prior to development the animal. Host cells with exogenous NFAT5/TonEBP can be used to produce non-human transgenic animals, such as fertilized oocytes or embryonic stem cells into which NFAT5/TonEBP-coding sequences have been introduced. Such host cells can then be used to create non-human transgenic animals or homologous recombinant animals.

Approaches to Transgenic Animal Production

A transgenic animal can be created by introducing NFAT5/TonEBP into the male pronuclei of a fertilized oocyte (e.g., by microinjection, retroviral infection) and allowing the oocyte to develop in a pseudopregnant female foster animal (pffa). The NFAT5/TonEBP sequences (SEQ ID NOs: 1, 3, 5, 7 or 9) can be introduced as a transgene into the genome of a non-human animal. Alternatively, a homologue of NFAT5/TonEBP can be used as a transgene. Intronic sequences and polyadenylation signals can also be included in the transgene to increase transgene expression. Tissue-specific regulatory sequences can be operably-linked to the NFAT5/TonEBP transgene to direct expression of NFAT5/TonEBP to particular cells. Methods for generating transgenic animals via embryo manipulation and microinjection, particularly animals such as mice, have become conventional in the art.

Other non-mice transgenic animals may be made by similar methods. A transgenic founder animal, which can be used to breed additional transgenic animals, can be identified based upon the presence of the transgene in its genome and/or expression of the transgene mRNA in tissues or cells of the animals. Transgenic (e.g., NFAT5/TonEBP) animals can be bred to other transgenic animals carrying other transgenes.

Vectors for Transgenic Animal Production

To create a homologous recombinant animal, a vector containing at least a portion of NFAT5/TonEBP into which a deletion, addition or substitution has been introduced to thereby alter, e.g., functionally disrupt, NFAT5/TonEBP. NFAT5/TonEBP can be a human gene (SEQ ID NOs: 1, 3, 5, 7 or 9), or other NFAT5/TonEBP homologue. In one approach, a knockout vector functionally disrupts the endogenous NFAT5/TonEBP gene upon homologous recombination, and thus a non-functional NFAT5/TonEBP protein, if any, is expressed.

Alternatively, the vector can be designed such that, upon homologous recombination, the endogenous NFAT5/TonEBP is mutated or otherwise altered but still encodes functional protein (e.g., the upstream regulatory region can be altered to thereby alter the expression of endogenous NFAT5/TonEBP). In this type of homologous recombination vector, the altered portion of the NFAT5/TonEBP is flanked at its 5′- and 3′-termini by additional nucleic acid of the NFAT5/TonEBP to allow for homologous recombination to occur between the exogenous NFAT5/TonEBP carried by the vector and an endogenous NFAT5/TonEBP in an embryonic stem cell. The additional flanking NFAT5/TonEBP nucleic acid is sufficient to engender homologous recombination with endogenous NFAT5/TonEBP. Typically, several kilobases of flanking DNA (both at the 5′- and 3′-termini) are included in the vector. The vector is then introduced into an embryonic stem cell line (e.g., by electroporation), and cells in which the introduced NFAT5/TonEBP has homologously-recombined with the endogenous NFAT5/TonEBP are selected.

Introduction of NFAT5/TonEBP Transgene Cells During Development

Selected cells are then injected into a blastocyst of an animal (e.g., a mouse) to form aggregation chimeras. A chimeric embryo can then be implanted into a suitable pffa and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described.

Alternatively, transgenic animals that contain selected systems that allow for regulated expression of the transgene can be produced. An example of such a system is the cre/IoxP recombinase system of bacteriophage P1. Another recombinase system is the FLP recombinase system of Saccharomyces cerevisiae. If a cre/IoxP recombinase system is used to regulate expression of the transgene, animals containing transgenes encoding both the Cre recombinase and a selected protein are required. Such animals can be produced as “double” transgenic animals, by mating an animal containing a transgene encoding a selected protein to another containing a transgene encoding a recombinase.

Clones of transgenic animals can also be produced. In brief, a cell from a transgenic animal can be isolated and induced to exit the growth cycle and enter Go phase. The quiescent cell can then be fused to an enucleated oocyte from an animal of the same species from which the quiescent cell is isolated. The reconstructed oocyte is then cultured to develop to a morula or blastocyte and then transferred to a pffa. The offspring borne of this female foster animal will be a clone of the “parent” transgenic animal.

In a further aspect of the present invention, a polynucleotide is provided comprising a nucleotide sequence selected from the group consisting of SEQ ID NO: 2, SEQ ID NO: 4, SEQ ID NO: 6, SEQ ID NO: 8, and SEQ ID NO: 10, and wherein the polynucleotide lacks exons 6 and 7 as described herein. In another aspect of the present invention, a polynucleotide is provided comprising at least about 80% sequence identity to a polynucleotide comprising a nucleotide sequence selected from the group consisting of SEQ ID NO: 2, SEQ ID NO: 4, SEQ ID NO: 6, SEQ ID NO: 8, and SEQ ID NO: 10, and wherein the polynucleotide encodes a polypeptide comprising at least one activity of an NFAT5/TonEBP protein.

In another aspect of the present invention, a polypeptide is provided which is expressed from the polynucleotide provided above. In another aspect of the present invention, a polypeptide is provided comprising an amino acid sequence selected from the group consisting of SEQ ID NO: 1, SEQ ID NO: 3, SEQ ID NO: 5, SEQ ID NO: 7, and SEQ ID NO: 9, and wherein the polypeptide lacks an amino acid sequence encoded by exons 6 and 7 of an NFAT5/TonEBP gene. In yet another aspect of the present invention, a polypeptide is provided having at least about 50% sequence identity to a polypeptide comprising an amino acid sequence selected from the group consisting of SEQ ID NO: 1, SEQ ID NO: 3, SEQ ID NO: 5, SEQ ID NO: 7, and SEQ ID NO: 9, and wherein the polypeptide comprises at least one activity of an NFAT5/TonEBP protein.

In a further aspect of the present invention, a vector is provided comprising the polynucleotide provided above. In another aspect, a cell is provided comprising any of the polynucleotides provided above. In yet another aspect, a tissue is provided comprising the cell provided above. In another aspect, an organism is provided comprising the cell provided above. In a further aspect, an organism is provided comprising a cell capable of expressing the polypeptides of any of the polypeptides provided above. NFAT5/TonEBP recombinant expression vectors and host cells

Vectors are tools used to shuttle DNA between host cells or as a means to express a nucleotide sequence. Some vectors function only in prokaryotes, while others function in both prokaryotes and eukaryotes, enabling large-scale DNA preparation from prokaryotes for expression in eukaryotes. Inserting the DNA of interest, such as NFAT5/TonEBP nucleotide sequence or a fragment, is accomplished by ligation techniques and/or mating protocols well known to the skilled artisan. Such DNA is inserted such that its integration does not disrupt any necessary components of the vector. In the case of vectors that are used to express the inserted DNA protein, the introduced DNA is operably-linked to the vector elements that govern its transcription and translation.

Vectors can be divided into two general classes: Cloning vectors are replicating plasmid or phage with regions that are non-essential for propagation in an appropriate host cell, and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA. In expression vectors, the introduced DNA is operably-linked to elements, such as promoters, that signal to the host cell to transcribe the inserted DNA. Some promoters are exceptionally useful, such as inducible promoters that control gene transcription in response to specific factors. Operably-linking NFAT5/TonEBP or anti-sense construct to an inducible promoter can control the expression of NFAT5/TonEBP or fragments, or anti-sense constructs. Examples of classic inducible promoters include those that are responsive to α-interferon, heat-shock, heavy metal ions, and steroids such as glucocorticoids and tetracycline. Other desirable inducible promoters include those that are not endogenous to the cells in which the construct is being introduced, but, however, is responsive in those cells when the induction agent is exogenously supplied.

Vectors have many difference manifestations. A “plasmid” is a circular double stranded DNA molecule into which additional DNA segments can be introduced. Viral vectors can accept additional DNA segments into the viral genome. Certain vectors are capable of autonomous replication in a host cell (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. In general, useful expression vectors are often plasmids. However, other forms of expression vectors, such as viral vectors (e.g., replication defective retroviruses, adenoviruses and adeno-associated viruses) are contemplated.

Recombinant expression vectors that comprise NFAT5/TonEBP (or fragments) regulate NFAT5/TonEBP transcription by exploiting one or more host cell-responsive (or that can be manipulated in vitro) regulatory sequences that is operably-linked to NFAT5/TonEBP. “Operably-linked” indicates that a nucleotide sequence of interest is linked to regulatory sequences such that expression of the nucleotide sequence is achieved.

Vectors can be introduced in a variety of organisms and/or cells (Table 5). Alternatively, the vectors can be transcribed and translated in vitro, for example using T7 promoter regulatory sequences and T7 polymerase.

TABLE 5
Examples of hosts for cloning or expression

| Organisms | Examples |
|---|---|
| Prokaryotes | |
| Enterobacteriaceae | E. coli |
| | K 12 strain MM294 |
| | X1776 |
| | W3110 |
| | K5 772 |
| | Enterobacter |
| | Erwinia |
| | Klebsiella |
| | Proteus |
| | Salmonella (S. tyhpimurium) |
| | Serratia (S. marcescans) |
| | Shigella |
| | Bacilli (B. subtilis and B. licheniformis) |
| | Pseudomonas (P. aeruginosa) |
| | Streptomyces |
| Eukaryotes | |
| Yeasts | Saccharomyces cerevisiae |
| | Schizosaccharomyces pombe |
| | Kluyveromyces |
| | K. lactis MW98-8C, CBS683, CBS4574 |
| | K. fragilis |
| | K. bulgaricus |
| | K. wickeramii |
| | K. waltii |
| | K. drosophilarum |
| | K. thermotolerans |
| | K. marxianus; yarrowia |
| | Pichia pastoris |
| | Candida |
| | Trichoderma reesia |
| | Neurospora crassa |
| | Torulopsis |
| | Rhodotorula |
| | Schwanniomyces (S. occidentalis) |
| Filamentous Fungi | Neurospora |
| | Penicillium |
| | Tolypocladium |
| | Aspergillus (A. nidulans and A. niger) |
| Invertebrate cells | Drosophila S2 |
| | Spodoptera Sf9 |
| Vertebrate cells | Chinese Hamster Ovary (CHO) |
| | simian COS |
| | COS-7 |
| | HEK 293 |

Vector choice is dictated by the organism or cells being used and the desired fate of the vector. Vectors may replicate once in the target cells, or may be “suicide” vectors. In general, vectors comprise signal sequences, origins of replication, marker genes, enhancer elements, promoters, and transcription termination sequences. The choice of these elements depends on the organisms in which the vector will be used and are easily determined. Some of these elements may be conditional, such as an inducible or conditional promoter that is turned “on” when conditions are appropriate. Examples of inducible promoters include those that are tissue-specific, which relegate expression to certain cell types, steroid-responsive, or heat-shock reactive. Some bacterial repression systems, such as the lac operon, have been exploited in mammalian cells and transgenic animals. Vectors often use a selectable marker to facilitate identifying those cells that have incorporated the vector. Many selectable markers are well known in the art for the use with prokaryotes, usually antibiotic-resistance genes or the use of autotrophy and auxotrophy mutants.

Antisense and Sense NFAT5/TonEBP Oligonucleotides

Using antisense and sense NFAT5/TonEBP oligonucleotides can prevent NFAT5/TonEBP polypeptide expression. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are singe-stranded nucleic acids, either RNA or DNA, which can bind target NFAT5/TonEBP mRNA (sense) or NFAT5/TonEBP DNA (antisense) sequences. According to the present invention, antisense or sense oligonucleotides comprise a fragment of the NFAT5/TonEBP DNA coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Methods to derive antisense or a sense oligonucleotides from a given cDNA sequence are well known in the art.

Modifications of antisense and sense oligonucleotides can augment their effectiveness. Modified sugar-phosphodiester bonds or other sugar linkages increase in vivo stability by conferring resistance to endogenous nucleases without disrupting binding specificity to target sequences. Other modifications can increase the affinities of the oligonucleotides for their targets, such as covalently linked organic moieties or poly-(L)-lysine. Other attachments modify binding specificities of the oligonucleotides for their targets, including metal complexes or intercalating (e.g., ellipticine) and alkylating agents.

Introduction of Antisense or Sense Oligonucleotides into Target Cells

To introduce antisense or sense oligonucleotides into target cells (cells containing the target nucleic acid sequence), any gene transfer method may be used and are well known to those of skill in the art. Examples of gene transfer methods include 1) biological, such as gene transfer vectors like Epstein-Barr virus or conjugating the exogenous DNA to a ligand-binding molecule, 2) physical, such as electroporation, and 3) chemical, such as CaPO₄ precipitation and oligonucleotide-lipid complexes.

The terms “host cell” and “recombinant host cell” are used interchangeably. Such terms refer not only to a particular subject cell but also to the progeny or potential progeny of such a cell. Because certain modifications may occur in succeeding generations due to either mutation or environmental influences, such progeny may not, in fact, be identical to the parent cell, but are still included within the scope of the term.

Methods of eukaryotic cell transfection and prokaryotic cell transformation are well known in the art. The choice of host cell will dictate the preferred technique for introducing the nucleic acid of interest. Table 6, which is not meant to be limiting, summarizes many of the known techniques in the art. Introduction of nucleic acids into an organism may also be done with ex vivo techniques that use an in vitro method of transfection, as well as established genetic techniques, if any, for that particular organism.

TABLE 6
Methods to introduce nucleic acid into cells

| Cells | Methods |
|---|---|
| Prokaryotes | Calcium chloride |
| (bacteria) | Electroporation |
| Eukaryotes | Calcium phosphate transfection |
| Mammalian | Diethylaminoethyl (DEAE)-Dextran transfection |
| cells | Electroporation |
| | Cationic lipid reagent transfection |
| | Retroviral |
| | Polybrene |
| | Microinjection |
| | Protoplast fusion |
| Insect cells | Baculovirus systems |
| (in vitro) | |
| Yeast | Electroporation |
| | Lithium acetate |
| | Spheroplast fusion |
| Plant cells | Agrobacterium transformation |
| | Biolistics (microprojectiles) |
| | Electroporation (protoplasts) |
| | Polyethylene glycol (PEG) treatment |
| | Liposomes |
| | in planta microinjection |
| | Seed imbibition |
| | Laser beam |
| | Silicon carbide whiskers |

Vectors often use a selectable marker to facilitate identifying those cells that have incorporated the vector. Many selectable markers are well known in the art for the use with prokaryotes, usually antibiotic-resistance genes or the use of autotrophy and auxotrophy mutants. Table 7 lists often-used selectable markers for mammalian cell transfection.

TABLE 7
Useful selectable markers for eukaryote cell transfection

| Selectable Marker | Selection | Action |
|---|---|---|
| Adenosine deaminase | Media includes 9-β-D- | Conversion of Xyl-A to Xyl-ATP, |
| (ADA) | xylofuranosyl adenine (Xyl- | which incorporates into nucleic |
| | A) | acids, killing cells. ADA detoxifies |
| Dihydrofolate reductase | Methotrexate (MTX) and | MTX competitive inhibitor of |
| (DHFR) | dialyzed serum (purine-free | DHFR. In absence of exogenous |
| | media) | purines, cells require DHFR, a |
| | | necessary enzyme in purine |
| | | biosynthesis. |
| Aminoglycoside | G418 | G418, an aminoglycoside |
| phosphotransferase | | detoxified by APH, interferes with |
| (“APH”, “neo”, “G418“) | | ribosomal function and |
| | | consequently, translation. |
| Hygromycin-B- | hygromycin-B | Hygromycin-B, an aminocyclitol |
| phosphotransferase | | detoxified by HPH, disrupts |
| (HPH) | | protein translocation and |
| | | promotes mistranslation. |
| Thymidine kinase (TK) | Forward selection (TK+): | Forward: Aminopterin forces cells |
| | Media HAT incorporates | to synthesze dTTP from |
| | aminopterin. | thymidine, a pathway requiring |
| | Reverse selection (TK−): | TK. |
| | Media incorporates 5- | Reverse: TK phosphorylates |
| | bromodeoxyuridine (BrdU). | BrdU, which incorporates into |
| | | nucleic acids, killing cells. |

A host cell of the invention, such as a prokaryotic or eukaryotic host cell in culture can be used to produce NFAT5/TonEBP. Accordingly, the invention provides methods for producing NFAT5/TonEBP using the host cells of the invention. In one embodiment, the method comprises culturing the host cell of the invention (into which a recombinant expression vector encoding NFAT5/TonEBP has been introduced) in a suitable medium, such that NFAT5/TonEBP is produced. In another embodiment, the method further comprises isolating NFAT5/TonEBP from the medium or the host cell.

In a further aspect of the present invention, an anti-cancer or immunosuppressive compound is provided which is identified by the method comprising: (a) culturing a first cell and a second cell under conditions of hypertonic stress; (b) contacting the first cell with a test agent; and (c) assaying the first cell and the second cell for cell growth or viability, wherein a decrease in cell growth or viability in the first cell relative to the second cell indicates that the test agent is an anti-cancer or immunosuppressive compound. In various aspects, the first and second cell can comprise an NFAT5/TonEBP polynucleotide or polypeptide as described above. In various aspects, the first and second cell comprise a NFAT5/TonEBP variant polynucleotide as described above. In various aspects, the first and second cell comprise a NFAT5/TonEBP variant or fragment protein as described above. In other aspects, the assay for cell growth or viability can comprise an assay selected from the group consisting of a cell uptake assay, cell binding assay, growth inhibition assay and any combination thereof as described above.

In yet another aspect of the present invention, an anti-cancer or immunosuppressive compound is provided which is identified by the method comprising: (a) performing a structure based drug design using a three dimensional structure determined for a crystal of NFAT5/TonEBP. In various aspects, the three dimensional structure can comprise atomic coordinates of Protein Data Bank Accession No. 1IMH as described above. In some aspects, the method can further comprise: (b) contacting the candidate inhibitor with a cell comprising NFAT5/TonEBP or a variant or fragment thereof; and (c) detecting inhibition of at least one activity of NFAT5/TonEBP, variant or fragment thereof as described above. Alternatively, the method can further comprise: (b) contacting the candidate inhibitor with a cell comprising NFAT5/TonEBP or a variant or fragment thereof; (c) culturing the cell under conditions of hypertonic stress; (d) contacting the cell with the candidate compound; and (e) assaying the cell for cell growth or viability as described above. In various aspects, the assay for cell growth or viability can comprise an assay selected from the group consisting of a cell uptake assay, cell binding assay, growth inhibition assay and any combination thereof as described above.

In various embodiments, the compounds identified above can comprise a pro-drug, pharmaceutically acceptable salt and can combine a pharmaceutically acceptable carrier.

Pharmaceutical Preparations and Methods of Administration

The identified compositions treat, inhibit, control and/or prevent, or at least partially arrest or partially prevent, diseases associated with osmotic stress and can be administered to a subject at therapeutically effective doses for the inhibition, prevention, prophylaxis or therapy for damage caused by such diseases. The compositions of the present invention comprise a therapeutically effective dosage of an antibody, antisense nucleic acid, a ribozyme, a triplex-forming molecule, a siRNA, an aptamer, and any combination thereof, and other compounds which suppress the expression of NFAT5/TonEBP protein, a term which includes therapeutically, inhibitory, preventive and prophylactically effective doses of the compositions of the present invention and is more particularly defined below. The subject is preferably an animal, including, but not limited to, mammals, reptiles and avians, more preferably horses, cows, dogs, cats, sheep, pigs, and chickens, and most preferably human.

Therapeutically Effective Dosage

Toxicity and therapeutic efficacy of such compositions can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀. Compositions that exhibit large therapeutic indices are preferred. While compositions exhibiting toxic side effects may be used, care should be taken to design a delivery system that targets such compositions to the site affected by the disease or disorder in order to minimize potential damage to unaffected cells and reduce side effects.

The data obtained from the cell culture assays and animal studies can be used in formulating a range of dosages for use in humans and other mammals. The dosage of such compositions lies preferably within a range of circulating plasma or other bodily fluid concentrations that include the ED₅₀ with little or no toxicity. The dosage may vary within this range depending upon the dosage form employed and the route of administration utilized. For any composition of the invention, the therapeutically effective dose can be estimated initially from cell culture assays. A dosage may be formulated in animal models to achieve a circulating plasma concentration range that includes the IC₅₀ (the concentration of the test composition that achieves a half-maximal inhibition of symptoms) as determined in cell culture. Such information can be used to more accurately determine useful dosages in humans and other mammals. Composition levels in plasma may be measured, for example, by high performance liquid chromatography.

The amount of a composition that may be combined with pharmaceutically acceptable carriers to produce a single dosage form will vary depending upon the host treated and the particular mode of administration. It will be appreciated by those skilled in the art that the unit content of a composition contained in an individual dose of each dosage form need not in itself constitute a therapeutically effective amount, as the necessary therapeutically effective amount could be reached by administration of a number of individual doses. The selection of dosage depends upon the dosage form utilized, the condition being treated, and the particular purpose to be achieved according to the determination of those skilled in the art.

The dosage regime for treating a disease or condition with the compositions and/or composition combinations of this invention is selected in accordance with a variety of factors, including the type, age, weight, sex, diet and medical condition of the patient, the route of administration, pharmacological considerations such as activity, efficacy, pharmacokinetic and toxicology profiles of the particular composition employed, whether a composition delivery system is utilized and whether the composition is administered as a pro-drug or part of a drug combination. Thus, the dosage regime actually employed may vary widely from subject to subject.

Formulations and Use

The compositions of the present invention may be formulated by known methods for administration to a subject using several routes which include, but are not limited to, parenteral, oral, topical, intradermal, intramuscular, intraperitoneal, intravenous, subcutaneous, intranasal, epidural, and ophthalmic routes. The individual compounds may also be administered in combination with one or more additional compounds of the present invention and/or together with other biologically active or biologically inert agents (“compositions” or “combinations”). Such biologically active or inert agents may be in fluid or mechanical communication with the composition(s) or attached to the composition(s) by ionic, covalent, Van der Waals, hydrophobic, hydrophilic or other physical forces. It is preferred that administration is localized in a subject, but administration may also be systemic.

The compounds or combinations may be formulated by any conventional manner using one or more pharmaceutically acceptable carriers and/or excipients. Thus, the compounds and their pharmaceutically acceptable salts and solvates may be specifically formulated for administration, e.g., by inhalation or insufflation (either through the mouth or the nose) or oral, buccal, parenteral or rectal administration. The composition or composition combinations may take the form of charged, neutral and/or other pharmaceutically acceptable salt forms. Examples of pharmaceutically acceptable carriers include, but are not limited to, those described in Remington's Pharmaceutical Sciences (A. R. Gennaro, Ed.), 20th edition, Williams & Wilkins PA, USA (2000).

The compounds may also take the form of solutions, suspensions, emulsions, tablets, pills, capsules, powders, controlled- or sustained-release formulations and the like. Such compositions will contain a therapeutically effective amount of the composition, preferably in purified form, together with a suitable amount of carrier so as to provide the form for proper administration to the patient. The formulation should suit the mode of administration.

Parenteral Administration

The compound or combination may be formulated for parenteral administration by injection, e.g., by bolus injection or continuous infusion. Formulations for injection may be presented in unit dosage form in ampoules or in multi-dose containers with an optional preservative added. The parenteral preparation can be enclosed in ampoules, disposable syringes or multiple dose vials made of glass, plastic or the like. The compound may take such forms as suspensions, solutions or emulsions in oily or aqueous vehicles, and may contain formulatory agents such as suspending, stabilizing and/or dispersing agents.

For example, a parenteral preparation may be a sterile injectable solution or suspension in a nontoxic parenterally acceptable diluent or solvent (e.g., as a solution in 1,3-butanediol). Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid may be used in the parenteral preparation.

Alternatively, the compound may be in powder form for constitution with a suitable vehicle, such as sterile pyrogen-free water, before use. For example, a compound suitable for parenteral administration may comprise a sterile isotonic saline solution containing between 0.1 percent and 90 percent weight per volume of the compound or combination. By way of example, a solution may contain from about 5 percent to about 20 percent, more preferably from about 5 percent to about 17 percent, more preferably from about 8 to about 14 percent, and still more preferably about 10 percent of the compound. The solution or powder preparation may also include a solubilizing agent and a local anesthetic such as lignocaine to ease pain at the site of the injection. Other methods of parenteral delivery of compounds will be known to the skilled artisan and are within the scope of the invention.

Oral Administration

For oral administration, the compound or combination may take the form of tablets or capsules prepared by conventional means with pharmaceutically acceptable excipients such as binding agents, fillers, lubricants and disintegrants:

A. Binding Agents

Binding agents include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Suitable forms of microcrystalline cellulose include, for example, the materials sold as AVICEL-PH-101, AVICEL-PH-103 and AVICEL-PH-105 (available from FMC Corporation, American Viscose Division, Avicel Sales, Marcus Hook, Pennsylvania, USA). An exemplary suitable binder is a mixture of microcrystalline cellulose and sodium carboxymethyl cellulose sold as AVICEL RC-581 by FMC Corporation.

B. Fillers

Fillers include, but are not limited to, talc, calcium carbonate (e.g., granules or powder), lactose, microcrystalline cellulose, powdered cellulose, dextrates, kaolin, mannitol, silicic acid, sorbitol, starch, pre-gelatinized starch, and mixtures thereof.

C. Lubricants

Lubricants include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, and mixtures thereof. Additional lubricants include, for example, a syloid silica gel (AEROSIL 200, manufactured by W.R. Grace Co. of Baltimore, Md., USA), a coagulated aerosol of synthetic silica (marketed by Deaussa Co. of Plano, Tex., USA), CAB-O-SIL (a pyrogenic silicon dioxide product sold by Cabot Co. of Boston, Mass., USA), and mixtures thereof.

D. Disintegrants

Disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

The tablets or capsules may optionally be coated by methods well known in the art. If binders and/or fillers are used with the compounds of the invention, they are typically formulated as about 50 to about 99 weight percent of the compound. Preferably, about 0.5 to about 15 weight percent of disintegrant, preferably about 1 to about 5 weight percent of disintegrant, may be used in the compound. A lubricant may optionally be added, typically in an amount of less than about 1 weight percent of the compound. Techniques and pharmaceutically acceptable additives for making solid oral dosage forms are described in Marshall, Solid Oral Dosage Forms, Modern Pharmaceutics (Banker and Rhodes, Eds.), 7:359-427 (1979). Other less typical formulations are known in the art.

Liquid preparations for oral administration may take the form of solutions, syrups or suspensions. Alternatively, the liquid preparations may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and/or preservatives (e.g., methyl or propyl-p-hydroxybenzoates or sorbic acid). The preparations may also contain buffer salts, flavoring, coloring, perfuming and sweetening agents as appropriate. Preparations for oral administration may also be formulated to achieve controlled release of the compound. Oral formulations preferably contain 10% to 95% compound. In addition, the compounds of the present invention may be formulated for buccal administration in the form of tablets or lozenges formulated in a conventional manner. Other methods of oral delivery of compounds will be known to the skilled artisan and are within the scope of the invention.

Controlled-Release Administration

Controlled-release (or sustained-release) preparations may be formulated to extend the activity of the compound or combination and reduce dosage frequency. Controlled-release preparations can also be used to effect the time of onset of action or other characteristics, such as blood levels of the compound, and consequently affect the occurrence of side effects.

Controlled-release preparations may be designed to initially release an amount of a compound that produces the desired therapeutic effect, and gradually and continually release other amounts of the compound to maintain the level of therapeutic effect over an extended period of time. In order to maintain a near-constant level of a compound in the body, the compound could be released from the dosage form at a rate that will replace the amount of compound being metabolized and/or excreted from the body. The controlled-release of a compound may be stimulated by various inducers, e.g., change in pH, change in temperature, enzymes, water, or other physiological conditions or molecules.

Controlled-release systems may include, for example, an infusion pump which may be used to administer the compound in a manner similar to that used for delivering insulin or chemotherapy to specific organs or tumors. Typically, using such a system, the compound is administered in combination with a biodegradable, biocompatible polymeric implant that releases the compound over a controlled period of time at a selected site. Examples of polymeric materials include polyanhydrides, polyorthoesters, polyglycolic acid, polylactic acid, polyethylene vinyl acetate, and copolymers and blends thereof. In addition, a controlled release system can be placed in proximity of a therapeutic target, thus requiring only a fraction of a systemic dosage.

The compounds of the invention may be administered by other controlled-release means or delivery devices that are well known to those of ordinary skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions. Other methods of controlled-release delivery of compounds will be known to the skilled artisan and are within the scope of the invention.

Inhalation Administration

The compound or combination may also be administered directly to the lung by inhalation. For administration by inhalation, a compound may be conveniently delivered to the lung by a number of different devices. For example, a Metered Dose Inhaler (“MDI”) which utilizes canisters that contain a suitable low boiling point propellant, e.g., dichlorodifluoromethane, trichlorofluoromethane, dichlorotetrafluoroethane, carbon dioxide or other suitable gas may be used to deliver a compound directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device may be used to administer a compound to the lung. DPI devices typically use a mechanism such as a burst of gas to create a cloud of dry powder inside a container, which may then be inhaled by the patient. DPI devices are also well known in the art and may be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator may be formulated containing a powder mix of the compound and a suitable powder base such as lactose or starch for these systems.

Another type of device that may be used to deliver a compound to the lung is a liquid spray device supplied, for example, by Aradigm Corporation. Liquid spray systems use extremely small nozzle holes to aerosolize liquid compound formulations that may then be directly inhaled into the lung. For example, a nebulizer device may be used to deliver a compound to the lung. Nebulizers create aerosols from liquid compound formulations by using, for example, ultrasonic energy to form fine particles that may be readily inhaled. Examples of nebulizers include devices supplied by Sheffield/Systemic Pulmonary Delivery Ltd., Aventis and Batelle Pulmonary Therapeutics.

In another example, an electrohydrodynamic (“EHD”) aerosol device may be used to deliver a compound to the lung. EHD aerosol devices use electrical energy to aerosolize liquid compound solutions or suspensions. The electrochemical properties of the compound formulation are important parameters to optimize when delivering this compound to the lung with an EHD aerosol device. Such optimization is routinely performed by one of skill in the art. Other methods of intra-pulmonary delivery of compounds will be known to the skilled artisan and are within the scope of the invention.

Liquid compound formulations suitable for use with nebulizers and liquid spray devices and EHD aerosol devices will typically include the compound with a pharmaceutically acceptable carrier. In one exemplary embodiment, the pharmaceutically acceptable carrier is a liquid such as alcohol, water, polyethylene glycol or a perfluorocarbon. Optionally, another material may be added to alter the aerosol properties of the solution or suspension of the compound. For example, this material may be a liquid such as an alcohol, glycol, polyglycol or a fatty acid. Other methods of formulating liquid compound solutions or suspensions suitable for use in aerosol devices are known to those of skill in the art.

Depot Administration

The compound or combination may also be formulated as a depot preparation. Such long-acting formulations may be administered by implantation (e.g., subcutaneously or intramuscularly) or by intramuscular injection. Accordingly, the compounds may be formulated with suitable polymeric or hydrophobic materials such as an emulsion in an acceptable oil or ion exchange resins, or as sparingly soluble derivatives such as a sparingly soluble salt. Other methods of depot delivery of compounds will be known to the skilled artisan and are within the scope of the invention.

Topical Administration

For topical application, the compound or combination may be combined with a carrier so that an effective dosage is delivered, based on the desired activity ranging from an effective dosage, for example, of 1.0 μM to 1.0 mM. In one embodiment, a topical compound is applied to the skin. The carrier may be in the form of, for example, and not by way of limitation, an ointment, cream, gel, paste, foam, aerosol, suppository, pad or gelled stick.

A topical formulation may also consist of a therapeutically effective amount of the compound in an opthalmologically acceptable excipient such as buffered saline, mineral oil, vegetable oils such as corn or arachis oil, petroleum jelly, Miglyol 182, alcohol solutions, or liposomes or liposome-like products. Any of these compounds may also include preservatives, antioxidants, antibiotics, immunosuppressants, and other biologically or pharmaceutically effective agents which do not exert a detrimental effect on the compound. Other methods of topical delivery of compounds will be known to the skilled artisan and are within the scope of the invention.

Suppository Administration

The compound or combination may also be formulated in rectal formulations such as suppositories or retention enemas containing conventional suppository bases such as cocoa butter or other glycerides and binders and carriers such as triglycerides, microcrystalline cellulose, gum tragacanth or gelatin. Suppositories may contain the compound in the range of 0.5% to 10% by weight. Other methods of suppository delivery of compounds will be known to the skilled artisan and are within the scope of the invention.

Gene Therapy Administration

The nucleic acid molecules of the invention can be inserted into vectors and used as gene therapy vectors. Gene therapy vectors can be delivered to a subject by, for example, intravenous injection, local administration (Nabel and Nabel, U.S. Pat. No. 5,328,470, 1994), or by stereotactic injection (Chen et al., 1994). The pharmaceutical preparation of a gene therapy vector can include an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Other Systems of Administration

Various other delivery systems are known in the art and can be used to administer the compounds of the invention. Moreover, these and other delivery systems may be combined and/or modified to optimize the administration of the compounds of the present invention. Exemplary formulations using the compounds of the present invention are described below (the compounds of the present invention are indicated as the active ingredient, but those of skill in the art will recognize that pro-drugs and compound combinations are also meant to be encompassed by this term):

Formulation 1—Hard gelatin capsules are prepared using the following ingredients:

TABLE 8

| Ingredients | mg/capsule |
|---|---|
| Active Ingredient | 250.0 |
| Starch | 305.0 |
| Magnesium stearate | 5.0 |

The above ingredients are mixed and filled into hard gelatin capsules in 560 mg quantities.

Formulation 2—A tablet formula is prepared using the following ingredients:

TABLE 9

| Ingredients | (mg/tablet) |
|---|---|
| Active Ingredient | 250.0 |
| Cellulose, microcrystalline | 400.0 |
| Colloidal silicon dioxide | 10.0 |
| Stearic acid | 5.0 |

The components are blended and compressed to form tablets, each weighing 665 mg.

Formulation 3—A dry powder inhaler formulation is prepared containing the following components:

TABLE 10

| Ingredients | Weight % |
|---|---|
| Active ingredient | 5 |
| Lactose | 95 |

The active ingredient is mixed with the lactose and the mixture is added to a dry powder inhaling appliance.

Formulation 4—Tablets, each containing 60 mg of active ingredient, are prepared as follows:

TABLE 11

| Ingredients | milligrams |
|---|---|
| Active ingredient | 60.0 |
| Starch | 45.0 |
| Microcrystalline cellulose | 35.0 |
| Polyvinylpyrrolidone (as 10% solution in water) | 4.0 |
| Sodium carboxymethyl starch | 4.5 |
| Magnesium stearate | 0.5 |
| Talc | 1.0 |
| Total | 150.0 |

The active ingredient, starch and cellulose are passed through a No. 20 mesh U.S. sieve and mixed thoroughly. The solution of polyvinylpyrrolidone is mixed with the resultant powders which are then passed through a 16 mesh U.S. sieve. The granules as produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 150 mg.

Formulation 5—Capsules, each containing 80 mg of active ingredient are made as follows:

TABLE 12

| Ingredients | milligrams |
|---|---|
| Active ingredient | 80.0 |
| Starch | 109.0 |
| Magnesium stearate | 1.0 |
| Total | 190.0 |

The active ingredient, cellulose, starch, and magnesium stearate are blended, passed through a No. 20 mesh U.S. sieve, and filled into hard gelatin capsules in 190 mg quantities.

Formulation 6-Suppositories, each containing 225 mg of active ingredient, are made as follows:

TABLE 13

| Ingredients | milligrams |
|---|---|
| Active Ingredient | 225 |
| Saturated fatty acid glycerides to | 2000 |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

Formulation 7-Suspensions, each containing 50 mg of active ingredient per 5.0 ml dose are made as follows:

TABLE 14

| Ingredients | | |
|---|---|---|
| Active ingredient | 50.0 | mg |
| Xanthan gum | 4.0 | mg |
| Sodium carboxymethyl cellulose | (11%) | |
| Microcrystalline cellulose | (89%) | |
| | 50.0 | mg |
| Sucrose | 1.75 | g |
| Sodium benzoate | 10.0 | mg |
| Flavor | q.v. | |
| Color | q.v. | |
| Purified water to | 5.0 | ml |

The active ingredient, sucrose and xanthan gum are blended, passed through a No. 10 mesh U.S. sieve, and mixed with a previously made solution of the microcrystalline cellulose and sodium carboxymethyl cellulose in water. The sodium benzoate, flavor, and color are diluted with some of the water and added with stirring. Sufficient water is then added to produce the required volume.

Formulation 8—Capsules, each containing 150 mg of active ingredient, are made as follows:

TABLE 15

| Ingredients | milligrams |
|---|---|
| Active ingredient | 150.0 |
| Starch | 407.0 |
| Magnesium stearate | 3.0 |
| Total | 560.0 |

The active ingredient, cellulose, starch, and magnesium stearate are blended, passed through a No. 20 mesh U.S. sieve, and filled into hard gelatin capsules in 560 mg quantities.

Packaging

The compounds may, if desired, be presented in a pack or dispenser device which may contain one or more unit dosage forms containing the compound. The pack may for example comprise metal or plastic foil, such as a blister pack. The pack or dispenser device may be accompanied by instructions for administration.

Kits, Research Reagents, Diagnostics, and Therapeutics

The NFAT5/TonEBP polynucleotides and NFAT5/TonEBP proteins identified herein can be utilized for diagnostics, therapeutics, prophylaxis, as research reagents and kits. Furthermore, antisense nucleic acid, a ribozyme, a triplex-forming oligonucleotide, a siRNA, an aptamer, a probe, a primer, and the like may be provided in a kit.

For use in kits and diagnostics, the NFAT5/TonEBP polynucleotides and proteins of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more NFAT5/TonEBP polynucleotides are compared to control cells or tissues not treated with antisense NFAT5/TonEBP polynucleotides and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression) (Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), fluorescent in situ hybridization (FISH) techniques (Going and Gusterson, Eur. J. Cancer, 1999, 35, 1895-904) and mass spectrometry methods (To, Comb. Chem. High Throughput Screen, 2000, 3, 235-41).

Further, the present invention provides a kit for detecting the progression of diseases of the invention. The kit comprises an NFAT5/TonEBP-specific antibody, whereby the detection of an illness can be carried out using the antibody in an assay as described above. The kit may comprise first and second antibodies specific to one or more NFAT5/TonEBP proteins. The second antibody is preferably capable of binding to a conjugate of the protein and the first antibody. For this purpose, for example, an antibody that recognizes an epitope different from that recognized by the first antibody may be used as the second antibody. It is provided that the first and second antibodies may be monoclonal antibodies.

The kit of the present invention may further comprise a substance and/or a device suitable for the detection of antibodies, the immobilization of antibodies, and the like. To immobilize the antibodies, the kit may further comprise a carrier (e.g., a microtiter plate), a solution for the immobilization (e.g., carbonate buffer) and a blocking solution (e.g., gelatin-containing phosphate buffered saline (PBS)). For the detection of the antibodies, it is preferable that the antibodies be labeled previously. In this case, the kit may further comprise a detecting reagent for detecting the label. For example, when biotin is used as the labeling substance, the detecting reagent may comprise a conjugate of streptavidin with horseradish peroxidase (HRP) as well as a color-developing solution that is capable of developing a color by the action of HRP.

The kit may further comprise instructions for performing the assays of the present invention in any media, including but not limited to paper, CD-ROM, via the Internet or other means of transmitting such instructions.

### EXAMPLES

Without further elaboration, it is believed that one skilled in the art can, using the preceding description, utilize the present invention to its fullest extent. The following specific examples are offered by way of illustration and not by way of limiting the remaining disclosure.

### Example 1

### Targeted Disruption of the Nfat5 Gene

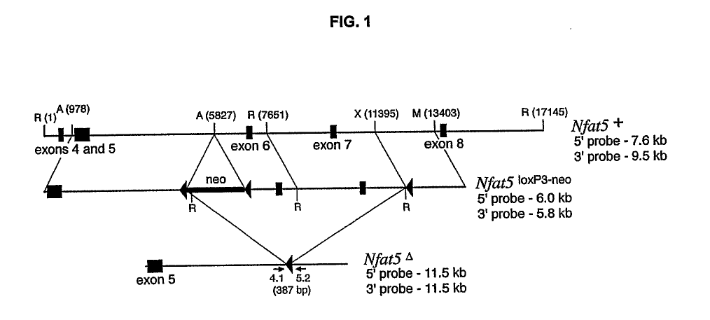

129/SvJ genomic DNA BAC clones (Research Genetics) encompassing the Nfat5 gene were utilized. The gene targeting vector used for homologous recombination in ES cells (FIG. 1) consisted of a 12.4 kilobase region of the Nfat5 gene encompassing exons 5 through 7. A neomycin resistance cassette flanked by IoxP recombination sites was inserted at the AvrII site located 4.8 kilobases from the 5′ end of the targeted genomic sequence, and an EcoRI-containing IoxP site was inserted at the XhoI site located 2.0 kilobases from the 3′ end. R1 (S129/SvJ) embryonic stem cells transfected with the targeting vector were grown and selected with G418.

Homologous recombinants were identified by Southern blotting of EcoRI-digested genomic DNA using 5′ and 3′ external probes that encompassed exons 4 and 8, respectively. Deletion of exons 6 and 7 was achieved by transfecting correctly targeted Nfat5^{Ioxp3-neo} ES cells with a cre recombinase expression vector and screening for cre-mediated recombination by Southern blotting using both the 5′ and 3′ external probes. The 884 bp 5′ probe was amplified from the genomic BAC clones containing the Nfat5 gene using primers SH272.1 (TTCGCTACCATACTGGAAAAGG; SEQ ID NO: 11) and SH272.2 (GATTTGTGAACTGATTGCTTTCC; SEQ ID NO: 12); the 672 bp 3′ probe was amplified using primers SH273.1 (ACACTCAAGAATCAGAGGCAGG; SEQ ID NO: 13) and SH273.2 (TCTTGTTTCTGCTCCTAGTCCC; SEQ ID NO: 14).

PCR genotyping was performed using two sets of primers that span the location of the downstream IoxP site, thus allowing PCR amplification from either the wildtype of knockout allele. The knockout allele was identified using primers SH274.1 (AACTTGCCCTGTCAGTCACC; SEQ ID NO: 15) and WG006.2 (GGGCTATAGACATGCACCACCACACAG; SEQ ID NO: 16); the wildtype allele was identified using primers SH275.1 (AAGGGCTTCTTCCAGAATGG; SEQ ID NO: 17) and WG006.2. Chimeric mice derived from blastocyst injections of gene-targeted Nfat5^{+/Δ} ES cells were crossed to C57BL6 and germline transmission was assessed by coat color.

### Example 2

### Generation of MEF Cell Lines

Primary mouse embryonic fibroblasts (MEFs) were obtained from E13.5 embryos generated from matings of heterozygous Nfat5^{+/Δ} mice that had been backcrossed once to C57BL6. The cells were cultured in complete media consisting of DMEM with high glucose supplemented with 10% fetal bovine serum, 100 U/ml penicillin, 100 ug/ml streptomycin, 2 mM L-glutamine and 10 mM HEPES, pH 7.4. Immortalized MEF cell lines were obtained by transfecting primary MEFs using the FuGene6 transfection reagent with an SV40 T antigen expression plasmid that confers resistance to neomycin, and culturing the cells in media containing 1 mg/ml G418. The immortalized MEF cells represent polyclonal cell lines. The cell lines were genotyped by both PCR and Southern blotting.

### Example 3

### Measurement of Tissue Osmolality

Tissue osmolality was measured using a vapor pressure osmometer (Wescor model 5520). Tissue obtained from anesthetized, 6-8 week old C57BL/6 mice was placed in a screw-cap microfuge tube and immediately frozen in a dry-ice/methanol bath. No more than two tissues were obtained per animal to minimize tissue ischemia. Blood samples were obtained by retro-orbital venipuncture. Osmolality measurements were made from filter discs adsorbed with tissue fluid from frozen tissue that had been fragmented.

Osmolality readings of standards were obtained immediately prior and subsequent to a tissue osmolality measurement to verify proper calibration of the instrument. In control experiments, measurement of either tissue fluid adsorbed onto a filter disk or tissue slices resulted in essentially identical results, and there was no difference in osmolality measurements comparing fresh and frozen tissue. Osmolality measurements of whole blood were essentially identical to those of serum.

### Example 4

### Lymphocyte Cell Culture

Purified splenocyte cell suspensions were prepared by density gradient centrifugation using Lympholyte M lymphocyte separation media (Cedarlane Laboratories, Hornby, Ontario) according to manufacturer specifications. Cells were cultured in complete media consisting of RPMI 1640 supplemented as above with the addition of 50 uM 2-mercaptoethanol. Cells were stimulated at a concentration of 1×10⁶ cells/ml with 1 ug/ml anti-TCR antibody (CD3 chain; clone 145-2C11), 25 ug/ml anti-CD28 antibody (clone 37.51; BD Biosciences, San Diego, Calif.), or with 25 ug/ml LPS (Calbiochem, San Diego, Calif.). Cells were pulsed with ³H-thymidine (0.5 uCi/well; Amersham, Piscataway, N.J.) during the final 12 hours of a 72 hour culture.

### Example 5

### Flow Cytometry

Flow cytometry was performed on a FACScan flow cytometer using CellQuest Software (BD Biosciences, Palo Alto, Calif.). Typically 40,000 total events or 20,000 gated events were analyzed. Monoclonal antibodies for flow cytometry were obtained from BD Biosciences (San Diego, Calif.).

### Example 6

### In Vivo Immune Response

Nfat5^{+/Δ} mice and Nfat5^{+/+} littermate controls (5-7 weeks old) were immunized subcutaneously with 50 μg ovalbumin (Sigma, St. Louis, Mo.) plus 5 μg LPS (Calbiochem, San Diego, Calif.) emulsified in incomplete Freund's adjuvant (Sigma, St. Louis, Mo.). Serum samples were obtained by retro-orbital venipuncture three weeks after immunization and antigen-specific immunoglobulin was measured by ELISA using an alkaline phosphatase-conjugated goat anti-mouse Ig kappa secondary antibody (clone, BD Biosciences, San Diego, Calif.) detected in a fluorescence-based assay.

### Example 7

### Western Immunoblot Analysis

Whole-cell lysates were subject to SDS-PAGE, transferred to PVDF membranes and probed with rabbit polyclonal antisera directed against the DNA binding domain (Trama et al., 2000), the N-terminus (Miyakawa et al., 1999) or the C-terminus (H. M. Kwon, University of Maryland, unpublished) of the NFAT5/TonEBP protein. Equal protein amounts were loaded based on determination of protein concentration by the Bradford dye-binding assay (Biorad). Primary antibody binding was visualized using an HRP-conjugated secondary antibody followed by enhanced chemiluminescence detection (Amersham, Arlington Heights, Ill.).

### Example 8

### Reporter Gene Analysis

Reporter studies were performed by transfecting MEF cell lines with either an NFAT5/TonEBP-responsive luciferase reporter gene (pGL3, Promega, Madison, Wis.) containing two tandem hTonE sites within a minimal promoter derived from the human IL2 gene or with a luciferase reporter gene in which transcription is directed by a 4.1-kilobase fragment of the mouse hsp70.1 gene promoter (referred to as HSP70-2).

### Example 9

### Impaired Immune Function in Nfat5^{+/Δ} Mutant Mice

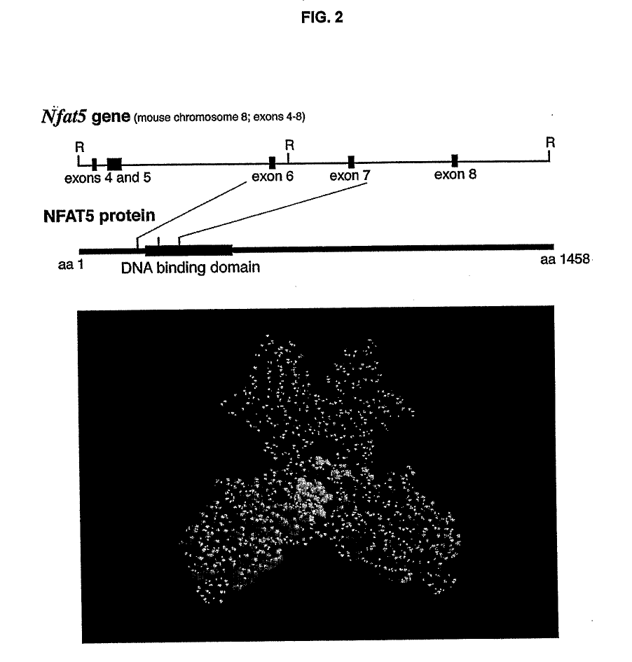

To define the biologic function of the NFAT5/TonEBP transcription factor in vivo, exons 6 and 7 of the murine Nfat5 gene were deleted by homologous recombination and cre recombinase-dependent excision in embryonic stem cells (FIG. 1). These exons encode amino acid residues 254-380, which comprise the amino-terminal portion of the DNA binding domain (DBD). This region is not only involved in critical base-specific contacts with DNA, but also forms one of two interfaces for dimerization within the DBD. The targeted deletion thus eliminates a region of the NFAT5/TonEBP protein that is essential to its function as a site-specific DNA binding transcription factor (FIG. 2). The structural depiction in the lower panel of FIG. 2 was generated using RASMOL; PDB 11H1, with the colors coded as follows: green=deleted amino acids, blue=remainder of the DNA-binding domain, and red and yellow=DNA.

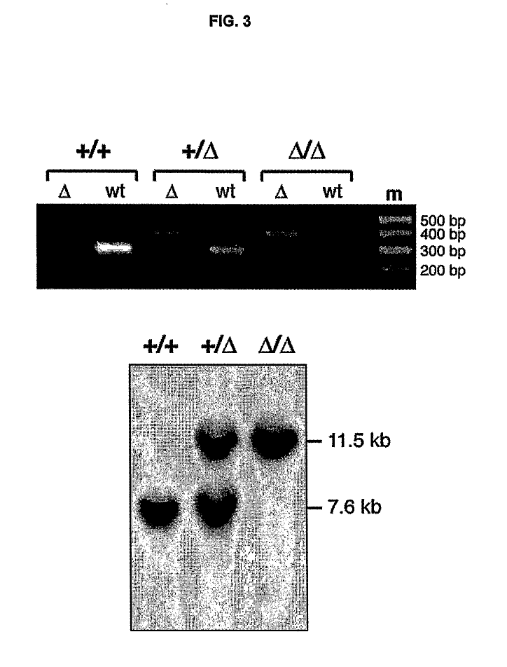

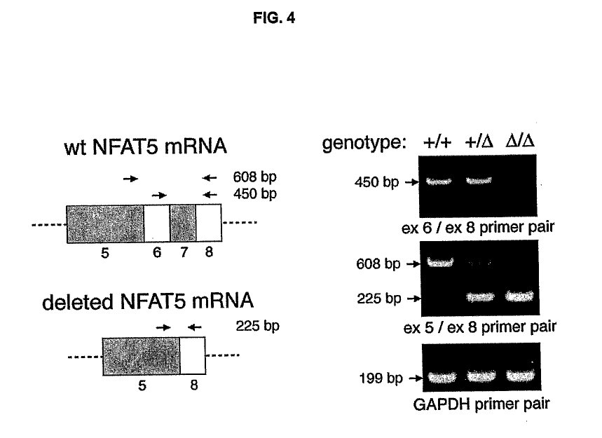

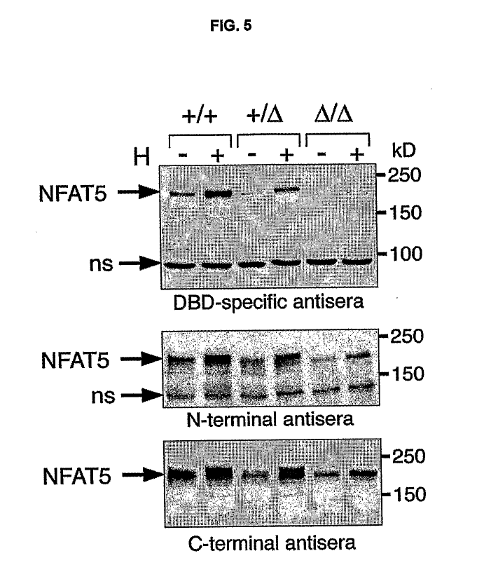

Germline transmission of the allele bearing the exon 6/7 deletion (hereafter referred to as Nfat5^{Δ}) was verified by PCR and Southern genotyping (FIG. 3). Analysis of NFAT5/TonEBP RNA and protein expression in Nfat5^{+/+} Nfat5^{+/Δ} and Nfat5^{Δ/Δ} mouse embryonic fibroblast (MEF) cell lines demonstrated that the Nfat5^{Δ} allele encodes a mutant protein containing an internal deletion of amino acid residues encoded by exons 6 and 7 (NFAT5/TonEBPΔ254-380), as expected based on the contiguous reading frame maintained between exons 5 and 8 (FIGS. 4 and 5). Targeted deletion of exons 6 and 7 was accomplished using two sets of primers that span the site of insertion of the downstream IoxP site in both the wildtype and knockout alleles.

NFAT5/TonEBP immunoreactivity in Nfat5^{Δ/Δ} cells is nearly completely eliminated using antisera directed against the DNA binding domain, and both constitutive and hypertonicity-induced expression of wildtype NFAT5/TonEBP expression is significantly reduced in Nfat5^{+/Δ} cells (FIG. 5, upper panel). Moreover, antisera directed against either the amino or carboxy termini demonstrate that upon hypertonic stimulation the mutant protein, although induced in level of expression, does not undergo phosphorylation-dependent post-translational modification given the absence of any reduction in mobility in SDS-PAGE analysis (FIG. 5, lower panel). Whole-cell extracts from MEF cell lines were cultured under either standard or hypertonic (H; complete medium plus additional 120 mM NaCl) culture conditions for 16 hours. The extracts were subjected to immunoblot analysis with the indicated antisera. The indicated nonspecific (ns) bands functioned as internal controls for equal protein loading and transfer.

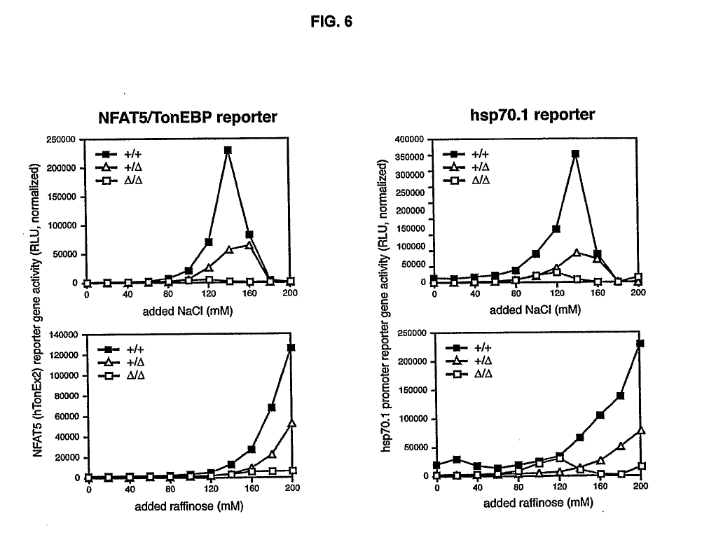

Based on deletion analysis of NFAT5/TonEBP dimerization and DNA binding function, the NFAT5/TonEBPΔ254-380 protein expressed in Nfat5^{+/Δ} cells likely functions to dominantly inhibit NFAT5/TonEBP function by forming dimers with wild type protein that are incapable of binding DNA in a sequence specific manner. To verify that the Nfat5^{Δ} allele conferred a loss of NFAT5/TonEBP function in not only the homozygous but also the heterozygous states, the MEF cell lines were transfected with an NFAT5 reporter gene and subject to culture under isotonic or hypertonic conditions (FIG. 6).

While Nfat5^{+/+} cells exhibited marked induction of NFAT5-dependent reporter gene expression upon culture in either NaCl or raffinose, Nfat5^{Δ/Δ} cells showed no induction, and Nfat5^{+/Δ} cells exhibited significant but incomplete loss of NFAT5/TonEBP-dependent reporter activity. Loss of NFAT5/TonEBP function was further demonstrated by measurement of transcription mediated by the hsp70.1 promoter, a previously defined NFAT5/TonEBP target gene (Woo et al., 2002). Remarkably, hypertonicity-induced reporter gene expression mediated by the hsp70.1 promoter was completely eliminated in Nfat5^{Δ/Δ} cells and markedly reduced in Nfat5^{+/Δ} cells (FIG. 6). SV40 T antigen-immortalized embryonic fibroblast (MEF) cell lines derived from Nfat5^{+/+}, Nfat5^{+/Δ}, Nfat5^{+/Δ} and Nfat5^{Δ/Δ} embryos were transfected with the indicated reporter gene. Approximately 24 hours after transfection the cells were cultured in complete media with either NaCl or raffinose added to the indicated concentration, and 16 hours later cell extracts were prepared for assay of reporter activity. The results represent luciferase reporter activity normalized to correct for variation in transfection efficiency, based on expression of a co-transfected constitutive secreted alkaline phosphatase reporter gene

Thus the invention provides for not only a complete loss of NFAT5/TonEBP transcriptional function resulting from homozygous deletion of exons 6 and 7, but also provides that the NFAT5/TonEBPΔ254-380 protein functions to dominantly inhibit NFAT5/TonEBP function in heterozygous Nfat5^{+/Δ} cells. In addition, that the invention provides that NFAT5/TonEBP is both necessary and sufficient for hypertonicity-dependent induction of the hsp70.1 promoter.

### Example 10

### Impaired Immune Function in Nfat5^{+/Δ} Mutant Mice

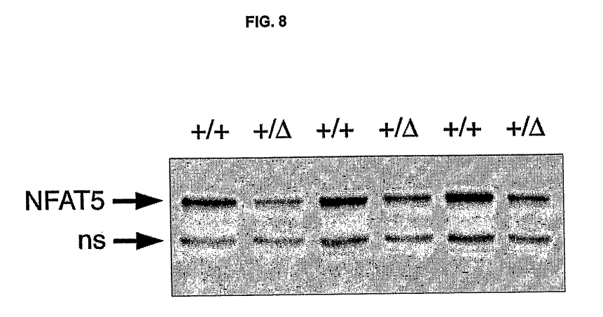

The genotype of litters obtained from matings of heterozygous Nfat5^{+/Δ} mice demonstrated that complete loss of NFAT5/TonEBP function due to homozygous deletion of exons 6 and 7 results in perinatal lethality, consistent with the similar timing of lethality observed in NFAT5/TonEBP null animals. The lethal phenotype associated with complete loss of NFAT5 function thus limits analysis of the role of NFAT5/TonEBP in regulating immune function. However, given that expression of the Nfat5^{Δ} allele confers partial loss of NFAT5/TonEBP-dependent transcriptional activity in the heterozygous state (FIG. 6), studies of immune function in heterozygous Nfat5^{+/Δ} animals were pursued. Remarkably, cellularity of the thymus and spleen from Nfat5^{+/Δ} animals was reduced by 40% and 32% relative to wildtype littermate controls (FIG. 7). Cell numbers were determined by manual cell counts using a hemocytometer. Viable cells were distinguished by trypan blue dye exclusion. Splenocytes were subject to density gradient centrifugation to remove red blood cells prior to counting. The observed phenotype is very similar to that of transgenic animals in which expression of a dominant negative form of NFAT5/TonEBP was targeted to T lymphocytes using the CD2 promoter, although the hypocellularity is significantly greater in the Nfat5^{+/Δ} mice. The observed reduction in cell number in thymuses from Nfat5^{+/Δ} animals correlated with reduced expression of the NFAT5/TonEB protein (FIG. 8). Whole cell extracts of thymocytes from Nfat5^{+/Δ} mice and Nfat5^{+/+} littermate controls were prepared and probed for NFAT5/TonEBP expression by SDS-PAGE Western blot analysis using the carboxy-terminal NFAT5/TonEBP antisera. The indicated non-specific (ns) band provides an internal control for equal protein loading and transfer.

Analysis of the composition of thymocyte subsets defined by the CD4 and CD8 markers of thymic development showed no differences (data not shown), again consistent with results obtained from the dominant negative NFAT5/TonEBP transgenic mice. In addition, there were no differences in the percentages of mature T and B cells within the spleens of wildtype and heterozygous animals (data not shown), indicating that the reduction in cell number was due to an equal reductions in the absolute number of both T and B cells.

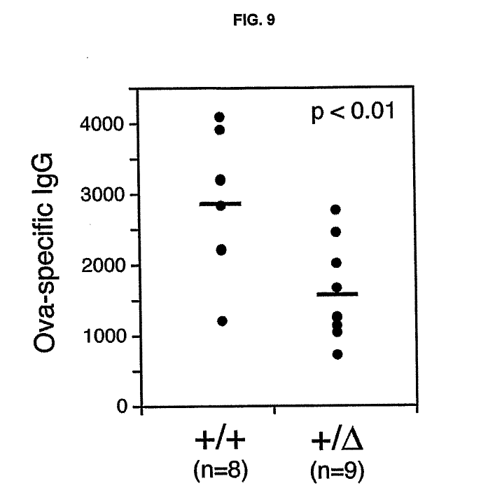

To determine whether partial loss of NFAT5/TonEBP function resulted in impaired lymphocyte function in vivo, a T cell-dependent B cell immune response was induced by immunization with the protein antigen ovalbumin. Heterozygous Nfat5^{+/Δ} animals were significantly impaired in their ability to mount an antigen-specific antibody response to this nominal protein antigen, exhibiting a 44% reduction in antigen-specific antibody as compared to wildtype controls (FIG. 9). Similarly reduced antibody responses were observed upon secondary immunizations (data not shown). In contrast, direct measurement of total serum IgG and IgM showed no difference between Nfat5^{+/+} and Nfat5^{+/Δ} animals, indicating that the impaired response by Nfat5^{+/Δ} animals does not simply reflect a difference in overall serum immunoglobulin concentration. The invention thus provides that a T cell-dependent B cell response in vivo is dependent upon the function of NFAT5/TonEBP.

### Example 11

### NFAT5/TonEBP and Cell Growth in a Hyperosmotic Environment

To determine whether the impaired lymphocyte-dependent responses observed in vivo were due to an inability to compensate for osmotic stress, proliferation of splenocytes from Nfat5^{+/+} and Nfat5^{+/Δ} animals was measured ex vivo under isotonic and hypertonic culture conditions. While there was no difference in the proliferative responses of T and B cells from Nfat5^{+/+} and Nfat5^{+/Δ} mice cultured under standard lymphocyte tissue culture conditions (i.e., 290 mOsm), proliferation was significantly impaired upon culture under hypertonic conditions (i.e., 370 mOsm; FIG. 10). The sensitivity of Nfat5^{+/Δ} T cell proliferation to hyperosmotic stress was not due to impairment in IL-2 production, as exogenously added IL-2 did not correct the defect (data not shown). Thus, the partial loss of NFAT5/TonEBP function conferred by the Nfat5^{Δ} allele results in an impaired osmotic stress response in lymphocytes in ex vivo cultures.

The effect of complete loss of NFAT5/TonEBP function on cell growth was determined by comparing the growth of immortalized Nfat5^{+/+}, Nfat5^{+/Δ} and Nfat5^{Δ/Δ} MEF cell lines under normal versus hyperosmotic culture conditions. Remarkably, while there were essentially no differences in cell growth under “normal” tissue culture conditions (˜300 mOsm), the growth of Nfat5^{Δ/Δ} MEF cells under hyperosmotic conditions were markedly impaired (FIG. 11, 12). The growth of heterozygous Nfat5^{+/Δ} MEF cells exhibited partial impairment relative to wild type cells, consistent with the partial loss of function demonstrated in NFAT5/TonEBP reporter gene studies (FIG. 6). The invention thus clearly provides that NFAT5/TonEBP function is essential for normal cell proliferation under conditions of hyperosmotic stress. Given the defect in lymphocyte proliferation observed upon partial loss of NFAT5/TonEBP function (FIG. 10), the invention potentially provides a complete loss of function in an even more markedly impaired lymphocyte response. Splenocytes from Nfat5^{+/Δ} mice and Nfat5^{+/+} littermate controls (5-8 weeks old) were cultured under isotonic (˜290 mOsm) conditions or subject to hypertonic stress (˜370 mOsm) through the addition of 80 mM raffinose to the culture media. The cells were stimulated with either anti-CD3 plus anti-CD28 to induce T cell proliferation or LPS to induce B cell proliferation. Proliferation was measured by quantitation of ³H-thymidine incorporation.

The defective proliferative response of Nfat5^{+/Δ} lymphocytes cultured ex vivo under conditions of hyperosmotic stress suggests that the impaired immune response observed in vivo is similarly due to an inability of lymphocytes to compensate or adapt to physiologic osmotic stress present within the lymphoid microenvironment. However, osmotic stress within the lymphoid microenvironment remains completely undefined. To specifically address this question, lymphoid tissue osmolality was measured directly by vapor pressure osmometry. Remarkably, in contrast to brain and lung, lymphoid tissues were significantly hyperosmolar relative to serum (FIG. 13). Hyperosmolality of lymphoid tissues relative to blood was determined by vapor pressure osmometry. Statistically significant differences between blood and tissue osmolality are indicated in FIG. 13 (*, p<0.02; **, p<0.0001). Liver tissue osmolality was also elevated. Thus the invention provides that lymphocytes are exposed to physiologic hyperosmotic stress. Given that partial loss of NFAT5/TonEBP function results in impaired lymphocyte function in vivo, it is likely that NFAT5/TonEBP functions to optimize lymphocyte function in vivo by regulating transcriptional programs that enhance the cells ability to compensate or adapt to osmotic stress present within the lymphoid microenvironment.

### Example 12

### NFAT5 is Essential for the Growth of Transformed Cells Under Hypertonic Culture Conditions

MEF cell lines were seeded in 24-well tissue culture dishes at 20,000 cells per well and allowed to grow until ˜80% confluence, at which time the cultures were continued by re-seeding at 20,000 cells per well. Manual cell counts using a hemocytometer were performed on the indicated days using trypan blue exclusion to identify viable cells. The culture media was replaced every three days. The data shown represent the mean of cell counts from triplicate wells. Standard errors, which were less than 5% of the mean, are not shown. These results are representative of at least four independent measurements of cell growth.

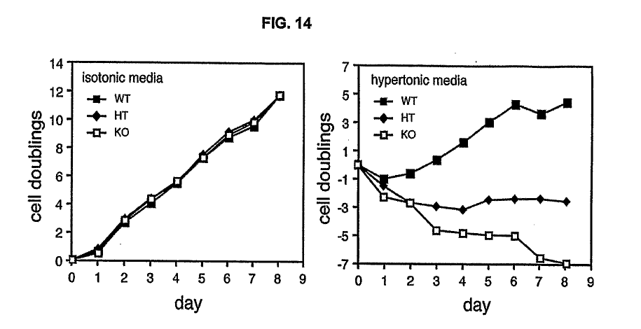

Primary MEF cells from wildtype (WT), heterozygous (HT) or homozygous (KO) embryos were transformed by stable transfection with a plasmid DNA vector directing the bicistronic expression of an oncogenic ras cDNA and a cDNA encoding the SV40 small and large T antigen, together with a neomycin-selectable marker. Neomycin-resistant cells were cultured using either normal (FIG. 14, left panel) or hypertonic (FIG. 14, right panel) tissue culture media. The cells were cultured at 20,000 cells per well in 24-well tissue culture dishes in either normal culture media (˜340 mOsm) or media supplemented with 100 mM NaCl (˜530-550 mOsm). At the indicated time points manual cell counts were performed on triplicate wells using trypan blue dye exclusion to quantitate viable cells. Individual wells, upon confluence, were split and the cells re-plated at 20,000 cells per well in a new plate on the following days: isotonic cultures—days 3 and 6; hypertonic cultures—day 7 for the wild type cell line only. Media was replaced every three days.

### Example 13

### NFAT5/TonEBP is Highly Expressed in Murine Prostate Carcinoma

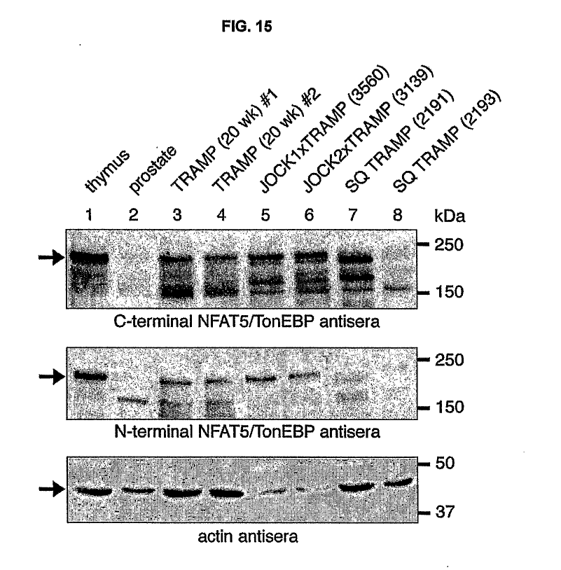

To determine expression of NFAT5/TonEBP in prostate carcinoma, normal and malignant prostate tissue was prepared by polytron disruption in RIPA buffer. A total of 50 μg of protein extract per sample was fractionated by SDS-PAGE, and replicate blots were probed as indicated (FIG. 15).

### Example 14

### Overexpression of NFAT5/TonEBP in Human Glioma Xenografts

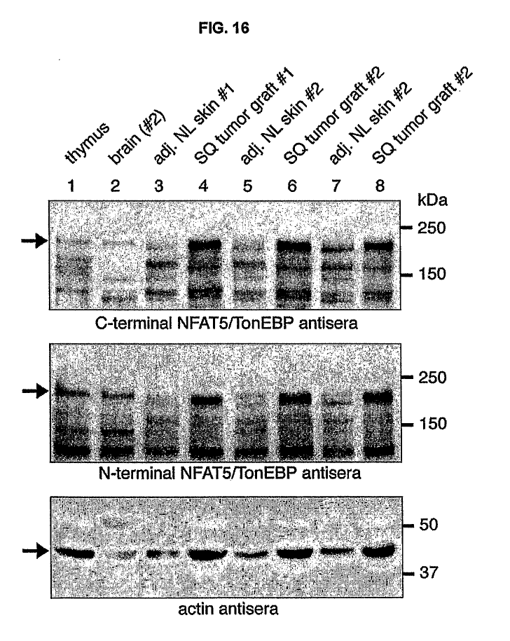

Protein extracts of tumor nodules (1 cm) from nude mice engrafted with U87 human glioma cancer cells were prepared by polytron disruption in RIPA buffer. A total of 50 μg of protein extract per sample was fractionated by SDS-PAGE, and replicate blots were probed as indicated (FIG. 16).

### Example 15

### NFAT5 is Over-Expressed in Human Cancer Tissue

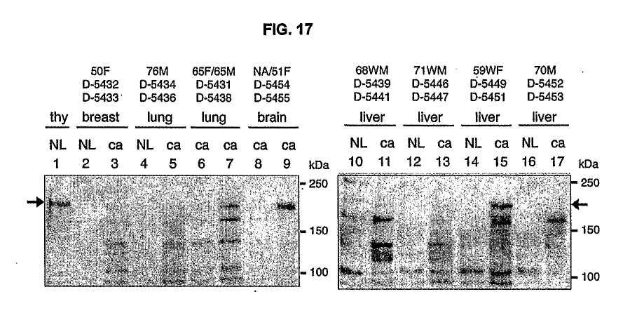

Normal and cancerous tissue from surgical resection specimen were obtained through the Cooperative Human Tissue Network (CHTN). Samples were analyzed for NFAT5 expression by Western blotting using the C-terminal NFAT5 antiserum. Similar results were obtained using the N-terminal NFAT5 antiserum (data not shown). Extract prepared from a murine thymocyte cell suspension was included (FIG. 17) as a control (lane 1, thy). The diagnoses of the samples analyzed include (FIG. 17): lane 3, infiltrating ductal carcinoma; lane 5, adenocarcinoma (AdCa), likely metastatic; lane 6, adenocarcinoma, unknown primary; lane 7, squamous cell carcinoma (SCC); lane 8, metastatic SCC from the lung; lane 9, metastatic poorly differentiated adenocarcinoma from the breast; lanes 11, 13, 15, 17, metastatic colorectal AdCa. NL, normal adjacent tissue; ca, cancer tissue; F, female; M, male. The CHTN identification numbers are listed together with the age, sex and race of the patient from whom the samples were obtained, if available. The arrows indicate the mobility of full length NFAT5. Multiple immunoreactive bands is likely a result of protein degradation resulting from prolonged time required to obtain and process surgically resected human tissue (note that similar degradation is not observed in murine tissues, which can be rapidly obtained and processed). The use of independent antisera directed against different regions of the NFAT5 protein validate the specificity of the observed reactivity.

### Example 16

### Tumor Growth In Vivo is Critically Dependent on NFAT5

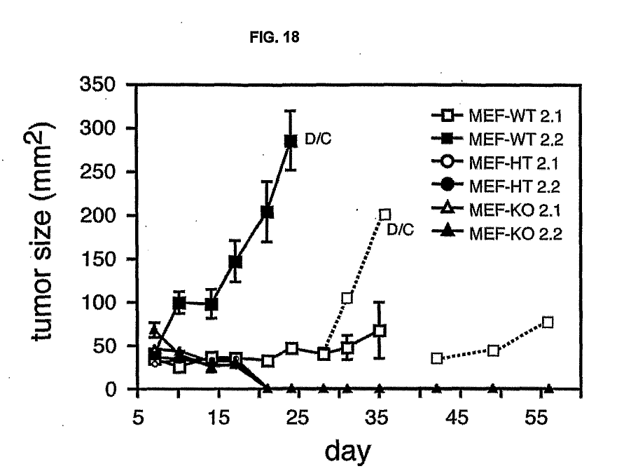

Male C57BL6 mice were injected subcutaneously with the indicated cell line (700 k cells per injection; 5 mice per group) and the formation and growth of tumor nodules was measured over time (FIG. 18). The data shown represent the mean tumor size (tumor nodule width, mm×length, m) with experimental variation shown as standard error of the mean. Mice with tumors larger than approximately 15×15 mm were euthanized (D/C). The open squares connected by a dashed line represent tumor growth in individual mice (i.e., while all five animals injected with the WT 2.2 line exhibited persistent, detectable tumor nodules, progressive tumor growth was observed in two of the five animals.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention. Specifically intended to be within the scope of the present invention, and incorporated herein by reference in its entirety, is the following publication: NFAT5/TonEBP mutant mice define osmotic stress as a critical feature of the lymphoid microenvironment, Proc. Natl. Acad. Sci. USA, 2004 Jul. 20; 101(29):10673-8.

1. A method for identifying a candidate anti-cancer or immunosuppressive compound, the method comprising:
(a) culturing a first cell and a second cell under conditions of hypertonic stress;
(b) contacting the first cell with a test agent; and
(c) assaying the first cell and the second cell for cell growth or viability,
wherein a decrease in cell growth or viability in the first cell relative to the second cell indicates that the test agent is a candidate anti-cancer or immunosuppressive compound.

2. A method according to claim 1, wherein the first and second cell comprise an NFAT5/TonEBP polynucleotide or polypeptide.

3. A method according to claim 2, wherein the first and second cell comprise a NFAT5/TonEBP variant polynucleotide

4-5. (canceled)

6. A method for identifying a candidate anti-cancer or immunosuppressive compound, the method comprising:
(a) contacting NFAT5/TonEBP or biologically-active fragment with a known compound that binds NFAT5/TonEBP to form an assay mixture,
(b) contacting the assay mixture with a test compound, and
(c) determining the ability of the test compound to interact with NFAT5/TonEBP.

7-11. (canceled)

12. A method for treating a cancer or an autoimmune disease in a subject, the method comprising administering to a subject in need thereof a therapeutically effective amount of an inhibitor of NFAT5/TonEBP, a pharmaceutically acceptable salt or pro-drug thereof, or combination with a pharmaceutically acceptable carrier.

13-17. (canceled)

18. A method according to claim 12, wherein the inhibitor is selected by:
(a) performing a structure based drug design using a three dimensional structure determined for a crystal of NFAT5/TonEBP.

19. (canceled)

20. A method according to claim 18, wherein the method further comprises:
(b) contacting the candidate inhibitor with a cell comprising NFAT5/TonEBP or a variant or fragment thereof; and
(c) detecting inhibition of at least one activity of NFAT5/TonEBP, variant or fragment thereof.

21. A method according to claim 18, wherein the method further comprises:
(b) contacting the candidate inhibitor with a cell comprising NFAT5/TonEBP or a variant or fragment thereof;
(c) culturing the cell under conditions of hypertonic stress;
(d) contacting the cell with the candidate compound; and
(e) assaying the cell for cell growth or viability.

22-26. (canceled)

27. A method according to claim 12, wherein the inhibitor is selected from the group consisting of a ribozyme, antisense compound, triplex-forming molecule, siRNA, and aptamer.

28. A method according to claim 12, wherein the cancer is selected from the group consisting of epithelial malignancies, sarcomas, and lymphomas.

29. A method according to claim 12, wherein the autoimmune disease is selected from the group consisting of rheumatoid arthritis, systemic lupus erythematosis, multiple sclerosis, and type I diabetes.

30. A method for diagnosing a cancer or an autoimmune disease in a subject comprising:
(a) obtaining a sample from the subject;
(b) detecting NFAT5/TonEBP expression in the sample; and
(c) comparing to the expression of NFAT5/TonEBP of the sample to a control sample,
wherein an elevated expression of NFAT5/TonEBP in the sample is diagnostic for cancer.

31-41. (canceled)

42. A method for determining whether a compound up-regulates or down-regulates the transcription of a NFAT5/TonEBP gene, comprising
contacting the compound with a RNA polymerase and said gene, followed by measuring NFAT5/TonEBP gene transcription initiated by the RNA polymerase acting on the gene, wherein measuring enhanced transcription is indicative of up-regulation and measuring decreased transcription is indicative of down-regulation.

43-46. (canceled)

47. A method for determining whether a compound is a NFAT5/TonEBP target gene inhibitor, comprising:
(a) culturing a first cell and a second cell under conditions of hypertonic stress;
(b) contacting the first cell with a test agent; and
(c) assaying the first cell and the second cell for at least one activity of a NFAT5/TonEBP target gene,
wherein a decrease in activity of a NFAT5/TonEBP target gene in the first cell relative to the second cell indicates that the test agent is an NFAT5/TonEBP target gene inhibitor.

48-50. (canceled)

51. A non-human transgenic animal, wherein at least one NFAT5/TonEBP gene comprised by the non-human transgenic animal is disrupted.

52-54. (canceled)

55. A polynucleotide comprising a nucleotide sequence selected from the group consisting of SEQ ID NO: 2, SEQ ID NO: 4, SEQ ID NO: 6, SEQ ID NO: 8, and SEQ ID NO: 10, and wherein the polynucleotide lacks exons 6 and 7.

56. A polynucleotide comprising at least about 80% sequence identity to a polynucleotide comprising a nucleotide sequence selected from the group consisting of SEQ ID NO: 2, SEQ ID NO: 4, SEQ ID NO: 6, SEQ ID NO: 8, and SEQ ID NO: 10, and wherein the polynucleotide encodes a polypeptide comprising at least one activity of an NFAT5/TonEBP protein.

57-64. (canceled)

65. An anti-cancer or immunosuppressive compound identified by the method comprising:
(a) culturing a first cell and a second cell under conditions of hypertonic stress;
(b) contacting the first cell with a test agent; and
(c) assaying the first cell and the second cell for cell growth or viability,
wherein a decrease in cell growth or viability in the first cell relative to the second cell indicates that the test agent is an anti-cancer or immunosuppressive compound.

66-69. (canceled)

70. An anti-cancer or immunosuppressive compound identified by the method comprising:
(a) performing a structure based drug design using a three dimensional structure determined for a crystal of NFAT5/TonEBP.

71-75. (canceled)
